SEQUENCE LISTING (50 sequences)

SEQ ID NO: 1 - DNA, 1356 bp, Homo sapiens
  CDS (1)..(1356)
atggagtcctcacccatcccccagtcatcagggaactcttccactttggggagggtccctcaaaccccaggtccctctactgccagtggggtcccggaggtggggctacgggatgttgcttcggaatctgtggccctcttcttcatgctcctgctggacttgactgctgtggctggcaatgccgctgtgatggccgtgatcgccaagacgcctgccctccgaaaatttgtcttcgtcttccacctctgcctggtggacctgctggctgccctgaccctcatgcccctggccatgctctccagctctgccctctttgaccacgccctctttggggaggtggcctgccgcctctacttgtttctgagcgtgtgctttgtcagcctggccatcctctcggtgtcagccatcaatgtggagcgctactattacgtagtccaccccatgcgctacgaggtgcgcatgacgctggggctggtggcctctgtgctggtgggtgtgtgggtgaaggccttggccatggcttctgtgccagtgttgggaagggtctcctgggaggaaggagctcccagtgtccccccaggctgttcactccagtggagccacagtgcctactgccagctttttgtggtggtctttgctgtcctttactttctgttgcccctgctcctcatacttgtggtctactgcagcatgttccgagtggcccgcgtggctgccatgcagcacgggccgctgcccacgtggatggagacaccccggcaacgctccgaatctctcagcagccgctccacgatggtcaccagctcgggggccccccagaccaccccacaccggacgtttgggggagggaaagcagcagtggttctcctggctgtggggggacagttcctgctctgttggttgccctacttctctttccacctctatgttgccctgagtgctcagcccatttcaactgggcaggtggagagtgtggtcacctggattggctacttttgcttcacttccaaccctttcttctatggatgtctcaaccggcagatccggggggagctcagcaagcagtttgtctgcttcttcaagccagctccagaggaggagctgaggctgcctagccgggagggctccattgaggagaacttcctgcagttccttcaggggactggctgtccttctgagtcctgggtttcccgacccctacccagccccaagcaggagccacctgctgttgactttcgaatcccaggccagatagctgaggagacctctgagttcctggagcagcaactcaccagcgacatcatcatgtcagacagctacctccgtcctgccgcctcaccccggctggagtcatga

SEQ ID NO: 2 - protein, 451 aa, Homo sapiens
MESSPIPQSSGNSSTLGRVPQTPGPSTASGVPEVGLRDVASESVALFFMLLLDLTAVAGNAAVMAVIAKTPALRKFVFVFHLCLVDLLAALTLMPLAMLSSSALFDHALFGEVACRLYLFLSVCFVSLAILSVSAINVERYYYVVHPMRYEVRMTLGLVASVLVGVWVKALAMASVPVLGRVSWEEGAPSVPPGCSLQWSHSAYCQLFVVVFAVLYFLLPLLLILVVYCSMFRVARVAAMQHGPLPTWMETPRQRSESLSSRSTMVTSSGAPQTTPHRTFGGGKAAVVLLAVGGQFLLCWLPYFSFHLYVALSAQPISTGQVESVVTWIGYFCFTSNPFFYGCLNRQIRGELSKQFVCFFKPAPEEELRLPSREGSIEENFLQFLQGTGCPSESWVSRPLPSPKQEPPAVDFRIPGQIAEETSEFLEQQLTSDIIMSDSYLRPAASPRLES

SEQ ID NO: 3 - DNA, 969 bp, Homo sapiens
  CDS (1)..(969)
atggatccaaccatctcaaccttggacacagaactgacaccaatcaacggaactgaggagactctttgctacaagcagaccttgagcctcacggtgctgacgtgcatcgtttcccttgtcgggctgacaggaaacgcagttgtgctctggctcctgggctgccgcatgcgcaggaacgccttctccatctacatcctcaacttggccgcagcagacttcctcttcctcagcggccgccttatatattccctgttaagcttcatcagtatcccccataccatctctaaaatcctctatcctgtgatgatgttttcctactttgcaggcctgagctttctgagtgccgtgagcaccgagcgctgcctgtccgtcctgtggcccatctggtaccgctgccaccgccccacacacctgtcagcggtggtgtgtgtcctgctctgggccctgtccctgctgcggagcatcctggagtggatgttatgtggcttcctgttcagtggtgctgattctgcttggtgtcaaacatcagatttcatcacagtcgcgtggctgatttttttatgtgtggttctctgtgggtccagcctggtcctgctgatcaggattctctgtggatcccggaagataccgctgaccaggctgtacgtgaccatcctgctcacagtactggtcttcctcctctgtggcctgccctttggcattcagtttttcctatttttatggatccacgtggacagggaagtcttattttgtcatgttcatctagtttctattttcctgtccgctcttaacagcagtgccaaccccatcatttacttcttcgtgggctcctttaggcagcgtcaaaataggcagaacctgaagctggttctccagagggctctgcaggacgcgtctgaggtggatgaaggtggagggcagcttcctgaggaaatcctggagctgtcgggaagcagattggagcagtga

SEQ ID NO: 4 - protein, 322 aa, Homo sapiens
MDPTISTLDTELTPINGTEETLCYKQTLSLTVLTCIVSLVGLTGNAVVLWLLGCRMRRNAFSIYILNLAAADFLFLSGRLIYSLLSFISIPHTISKILYPVMMFSYFAGLSFLSAVSTERCLSVLWPIWYRCHRPTHLSAVVCVLLWALSLLRSILEWMLCGFLFSGADSAWCQTSDFITVAWLIFLCVVLCGSSLVLLIRILCGSRKIPLTRLYVTILLTVLVFLLCGLPFGIQFFLFLWIHVDREVLFCHVHLVSIFLSALNSSANPIIYFFVGSFRQRQNRQNLKLVLQRALQDASEVDEGGGQLPEEILELSGSRLEQ

SEQ ID NO: 5 - DNA, 1017 bp, Homo sapiens
  CDS (1)..(1017)
atgctgtccattttgcttccttccaggggaagcagaagcgggagccgtcgtggagctctgctcctggagggagcctcccgggacatggagaaggtggacatgaatacatcacaggaacaaggtctctgccagttctcagagaagtacaagcaagtctacctctccctggcctacagtatcatctttatcctagggctgccactaaatggcactgtcttgtggcactcctggggccaaaccaagcgctggagctgtgccaccacctatctggtgaacctgatggtggccgacctgctttatgtgctattgcccttcctcatcatcacctactcactagatgacaggtggcccttcggggagctgctctgcaagctggtgcacttcctgttctatatcaacctttacggcagcatcctgctgctgacctgcatctctgtgcaccagttcctaggtgtgtgccacccactgtgttcgctgccctaccggacccgcaggcatgcctggctgggcaccagcaccacctgggccctggtggtcctccagctgctgcccacactggccttctcccacacggactacatcaatggccagatgatctggtatgacatgaccagccaagagaattttgatcggctttttgcctacggcatagttctgacattgtctggcttttttccctccttggtcattttggtgtgctattcactgatggtcaggagcctgatcaagccagaggagaacctcatgaggacaggcaacacagcccgagccaggtccatccggaccatcctactggtgtgtggcctcttcaccctctgttttgtgcccttccatatcactcgctccttctacctcaccatctgctttctgctttctcaggactgccagctcttgatggcagccagtgtggcctacaagatatggaggcctctggtgagtgtgagcagctgcctcaacccagtcctgtactttctttcaaggggggcaaaaatagagtcaggctcctccagaaactga

SEQ ID NO: 6 - protein, 338 aa, Homo sapiens
MLSILLPSRGSRSGSRRGALLLEGASRDMEKVDMNTSQEQGLCQFSEKYKQVYLSLAYSIIFILGLPLNGTVLWHSWGQTKRWSCATTYLVNLMVADLLYVLLPFLIITYSLDDRWPFGELLCKLVHFLFYINLYGSILLLTCISVHQFLGVCHPLCSLPYRTRRHAWLGTSTTWALVVLQLLPTLAFSHTDYINGQMIWYDMTSQENFDRLFAYGIVLTLSGFFPSLVILVCYSLMVRSLIKPEENLMRTGNTARARSIRTILLVCGLFTLCFVPFHITRSFYLTICFLLSQDCQLLMAASVAYKIWRPLVSVSSCLNPVLYFLSRGAKIESGSSRN

SEQ ID NO: 7 - DNA, 966 bp, Homo sapiens
  CDS (1)..(966)
atgaaccagactttgaatagcagtgggaccgtggagtcagccctaaactattccagagggagcacagtgcacacggcctacctggtgctgagctccctggccatgttcacctgcctgtgcgggatggcaggcaacagcatggtgatctggctgctgggctttcgaatgcacaggaaccccttctgcatctatatcctcaacctggcggcagccgacctcctcttcctcttcagcatggcttccacgctcagcctggaaacccagcccctggtcaataccactgacaaggtccacgagctgatgaagagactgatgtactttgcctacacagtgggcctgagcctgctgacggccatcagcacccagcgctgtctctctgtcctcttccctatctggttcaagtgtcaccggcccaggcacctgtcagcctgggtgtgtggcctgctgtggacactctgtctcctgatgaacgggttgacctcttccttctgcagcaagttcttgaaattcaatgaagatcggtgcttcagggtggacatggtccaggccgccctcatcatgggggtcttaaccccagtgatgactctgtccagcctgaccctctttgtctgggtgcggaggagctcccagcagtggcggcggcagcccacacggctgttcgtggtggtcctggcctctgtcctggtgttcctcatctgttccctgcctctgagcatctactggtttgtgctctactggttgagcctgccgcccgagatgcaggtcctgtgcttcagcttgtcacgcctctcctcgtccgtaagcagcagcgccaaccccgtcatctacttcctggtgggcagccggaggagccacaggctgcccaccaggtccctggggactgtgctccaacaggcgcttcgcgaggagcccgagctggaaggtggggagacgcccaccgtgggcaccaatgagatgggggcttga

SEQ ID NO: 8 - protein, 321 aa, Homo sapiens
MNQTLNSSGTVESALNYSRGSTVHTAYLVLSSLAMFTCLCGMAGNSMVIWLLGFRMHRNPFCIYILNLAAADLLFLFSMASTLSLETQPLVNTTDKVHELMKRLMYFAYTVGLSLLTAISTQRCLSVLFPIWFKCHRPRHLSAWVCGLLWTLCLLMNGLTSSFCSKFLKFNEDRCFRVDMVQAALIMGVLTPVMTLSSLTLFVWVRRSSQQWRRQPTRLFVVVLASVLVFLICSLPLSIYWFVLYWLSLPPEMQVLCFSLSRLSSSVSSSANPVIYFLVGSRRSHRLPTRSLGTVLQQALREEPELEGGETPTVGTNEMGA

SEQ ID NO: 9 - DNA, 1401 bp, Homo sapiens
  CDS (1)..(1401)
atggaagctgacctgggtgccactggccacaggccccgcacagagcttgatgatgaggactcctacccccaaggtggctgggacacggtcttcctggtggccctgctgctccttgggctgccagccaatgggttgatggcgtggctggccggctcccaggcccggcatggagctggcacgcgtctggcgctgctcctgctcagcctggccctctctgacttcttgttcctggcagcagcggccttccagatcctagagatccggcatgggggacactggccgctggggacagctgcctgccgcttctactacttcctatggggcgtgtcctactcctccggcctcttcctgctggccgccctcagcctcgaccgctgcctgctggcgctgtgcccacactggtaccctgggcaccgcccagtccgcctgcccctctgggtctgcgccggtgtctgggtgctggccacactcttcagcgtgccctggctggtcttccccgaggctgccgtctggtggtacgacctggtcatctgcctggacttctgggacagcgaggagctgtcgctgaggatgctggaggtcctggggggcttcctgcctttcctcctgctgctcgtctgccacgtgctcacccaggccacagcctgtcgcacctgccaccgccaacagcagcccgcagcctgccggggcttcgcccgtgtggccaggaccattctgtcagcctatgtggtcctgaggctgccctaccagctggcccagctgctctacctggccttcctgtgggacgtctactctggctacctgctctgggaggccctggtctactccgactacctgatcctactcaacagctgcctcagccccttcctctgcctcatggccagtgccgacctccggaccctgctgcgctccgtgctctcgtccttcgcggcagctctctgcgaggagcggccgggcagcttcacgcccactgagccacagacccagctagattctgagggtccaactctgccagagccgatggcagaggcccagtcacagatggatcctgtggcccagcctcaggtgaaccccacactccagccacgatcggatcccacagctcagccacagctgaaccctacggcccagccacagtcggatcccacagcccagccacagctgaacctcatggcccagccacagtcagattctgtggcccagccacaggcagacactaacgtccagacccctgcacctgctgccagttctgtgcccagtccctgtgatgaagcttccccaaccccatcctcgcatcctaccccaggggcccttgaggacccagccacacctcctgcctctgaaggagaaagccccagcagcaccccgccagaggcggccccgggcgcaggccccacgtga

SEQ ID NO: 10 - protein, 466 aa, Homo sapiens [sequence omitted]

SEQ ID NO: 11 - DNA, 993 bp, Homo sapiens
  CDS (1)..(993)
atggatccaaccaccccggcctggggaacagaaagtacaacagtgaatggaaatgaccaagcccttcttctgctttgtggcaaggagaccctgatcccggtcttcctgatccttttcattgccctggtcgggctggtaggaaacgggtttgtgctctggctcctgggcttccgcatgcgcaggaacgccttctctgtctacgtcctcagcctggccggggccgacttcctcttcctctgcttccagattataaattgcctggtgtacctcagtaacttcttctgttccatctccatcaatttccctagcttcttcaccactgtgatgacctgtgcctaccttgcaggcctgagcatgctgagcaccgtcagcaccgagcgctgcctgtccgtcctgtggcccatctggtatcgctgccgccgccccagacacctgtcagcggtcgtgtgtgtcctgctctgggccctgtccctactgctgagcatcttggaagggaagttctgtggcttcttatttagtgatggtgactctggttggtgtcagacatttgatttcatcactgcagcgtggctgatttttttattcatggttctctgtgggtccagtctggccctgctggtcaggatcctctgtggctccaggggtctgccactgaccaggctgtacctgaccatcctgctcacagtgctggtgttcctcctctgcggcctgccctttggcattcagtggttcctaatattatggatctggaaggattctgatgtcttattttgtcatattcatccagtttcagttgtcctgtcatctcttaacagcagtgccaaccccatcatttacttcttcgtgggctcttttaggaagcagtggcggctgcagcagccgatcctcaagctggctctccagagggctctgcaggacattgctgaggtggatcacagtgaaggatgcttccgtcagggcaccccggagatgtcgagaagcagtctggtgtag

SEQ ID NO: 12 - protein, 330 aa, Homo sapiens
MDPTTPAWGTESTTVNGNDQALLLLCGKETLIPVFLILFIALVGLVGNGFVLWLLGFRMRRNAFSVYVLSLAGADFLFLCFQIINCLVYLSNFFCSISINFPSFFTTVMTCAYLAGLSMLSTVSTERCLSVLWPIWYRCRRPRHLSAVVCVLLWALSLLLSILEGKFCGFLFSDGDSGWCQTFDFITAAWLIFLFMVLCGSSLALLVRILCGSRGLPLTRLYLTILLTVLVFLLCGLPFGIQWFLILWIWKDSDVLFCHIHPVSVVLSSLNSSANPIIYFFVGSFRKQWRLQQPILKLALQRALQDIAEVDHSEGCFRQGTPEMSRSSLV

SEQ ID NO: 13 - DNA, 1014 bp, Homo sapiens
  CDS (1)..(1014)
atgaatgagccactagactatttagcaaatgcttctgatttccccgattatgcagctgcttttggaaattgcactgatgaaaacatcccactcaagatgcactacctccctgttatttatggcattatcttcctcgtgggatttccaggcaatgcagtagtgatatccacttacattttcaaaatgagaccttggaagagcagcaccatcattatgctgaacctggcctgcacagatctgctgtatctgaccagcctccccttcctgattcactactatgccagtggcgaaaactggatctttggagatttcatgtgtaagtttatccgcttcagcttccatttcaacctgtatagcagcatcctcttcctcacctgtttcagcatcttccgctactgtgtgatcattcacccaatgagctgcttttccattcacaaaactcgatgtgcagttgtagcctgtgctgtggtgtggatcatttcactggtagctgtcattccgatgaccttcttgatcacatcaaccaacaggaccaacagatcagcctgtctcgacctcaccagttcggatgaactcaatactattaagtggtacaacctgattttgactgcaactactttctgcctccccttggtgatagtgacactttgctataccacgattatccacactctgacccatggactgcaaactgacagctgccttaagcagaaagcacgaaggctaaccattctgctactccttgcattttacgtatgttttttacccttccatatcttgagggtcattcggatcgaatctcgcctgctttcaatcagttgttccattgagaatcagatccatgaagcttacatcgtttctagaccattagctgctctgaacacctttggtaacctgttactatatgtggtggtcagcgacaactttcagcaggctgtctgctcaacagtgagatgcaaagtaagcgggaaccttgagcaagcaaagaaaattagttactcaaacaacccttga

SEQ ID NO: 14 - protein, 337 aa, Homo sapiens
MNEPLDYLANASDFPDYAAAFGNCTDENIPLKMHYLPVIYGIIFLVGFPGNAVVISTYIFKMRPWKSSTIIMLNLACTDLLYLTSLPFLIHYYASGENWIFGDFMCKFIRFSFHFNLYSSILFLTCFSIFRYCVIIHPMSCFSIHKTRCAVVACAVVWIISLVAVIPMTFLITSTNRTNRSACLDLTSSDELNTIKWYNLILTATTFCLPLVIVTLCYTTIIHTLTHGLQTDSCLKQKARRLTILLLLAFYVCFLPFHILRVIRIESRLLSISCSIENQIHEAYIVSRPLAALNTFGNLLLYVVVSDNFQQAVCSTVRCKVSGNLEQAKKISYSNNP

SEQ ID NO: 15 - DNA, 1011 bp, Homo sapiens
  CDS (1)..(1011)
atgaacaacaatacaacatgtattcaaccatctatgatctcttccatggctttaccaatcatttacatcctcctttgtattgttggtgtttttggaaacactctctctcaatggatatttttaacaaaaataggtaaaaaaacatcaacgcacatctacctgtcacaccttgtgactgcaaacttacttgtgtgcagtgccatgcctttcatgagtatctatttcctgaaaggtttccaatgggaatatcaatctgctcaatgcagagtggtcaattttctgggaactctatccatgcatgcaagtatgtttgtcagtctcttaattttaagttggattgccataagccgctatgctaccttaatgcaaaaggattcctcgcaagagactacttcatgctatgagaaaatattttatggccatttactgaaaaaatttcgccagcccaactttgctagaaaactatgcatttacatatggggagttgtactgggcataatcattccagttaccgtatactactcagtcatagaggctacagaaggagaagagagcctatgctacaatcggcagatggaactaggagccatgatctctcagattgcaggtctcattggaaccacatttattggattttcctttttagtagtactaacatcatactactcttttgtaagccatctgagaaaaataagaacctgtacgtccattatggagaaagatttgacttacagttctgtgaaaagacatcttttggtcatccagattctactaatagtttgcttccttccttatagtatttttaaacccattttttatgttctacaccaaagagataactgtcagcaattgaattatttaatagaaacaaaaaacattctcacctgtcttgcttcggccagaagtagcacagaccccattatatttcttttattagataaaacattcaagaagacactatataatctctttacaaagtctaattcagcacatatgcaatcatatggttga

SEQ ID NO: 16 - protein, 336 aa, Homo sapiens
MNNNTTCIQPSMISSMALPIIYILLCIVGVFGNTLSQWIFLTKIGKKTSTHIYLSHLVTANLLVCSAMPFMSIYFLKGFQWEYQSAQCRVVNFLGTLSMHASMFVSLLILSWIAISRYATLMQKDSSQETTSCYEKIFYGHLLKKFRQPNFARKLCIYIWGVVLGIIIPVTVYYSVIEATEGEESLCYNRQMELGAMISQIAGLIGTTFIGFSFLVVLTSYYSFVSHLRKIRTCTSIMEKDLTYSSVKRHLLVIQILLIVCFLPYSIFKPIFYVLHQRDNCQQLNYLIETKNILTCLASARSSTDPIIFLLLDKTFKKTLYNLFTKSNSAHMQSYG

SEQ ID NO: 17 - DNA, 1041 bp, Homo sapiens
  CDS (1)..(1041)
atgtacaacgggtcgtgctgccgcatcgagggggacaccatctcccaggtgatgccgccgctgctcattgtggcctttgtgctgggcgcactaggcaatggggtcgccctgtgtggtttctgcttccacatgaagacctggaagcccagcactgtttaccttttcaatttggccgtggctgatttcctccttatgatctgcctgccttttcggacagactattacctcagacgtagacactgggcttttggggacattccctgccgagtggggctcttcacgttggccatgaacagggccgggagcatcgtgttccttacggtggtggctgcggacaggtatttcaaagtggtccacccccaccacgcggtgaacactatctccacccgggtggcggctggcatcgtctgcaccctgtgggccctggtcatcctgggaacagtgtatcttttgctggagaaccatctctgcgtgcaagagacggccgtctcctgtgagagcttcatcatggagtcggccaatggctggcatgacatcatgttccagctggagttctttatgcccctcggcatcatcttattttgctccttcaagattgtttggagcctgaggcggaggcagcagctggccagacaggctcggatgaagaaggcgacccggttcatcatggtggtggcaattgtgttcatcacatgctacctgcccagcgtgtctgctagactctatttcctctggacggtgccctcgagtgcctgcgatccctctgtccatggggccctgcacataaccctcagcttcacctacatgaacagcatgctggatcccctggtgtattatttttcaagcccctcctttcccaaattctacaacaagctcaaaatctgcagtctgaaacccaagcagccaggacactcaaaaacacaaaggccggaagagatgccaatttcgaacctcggtcgcaggagttgcatcagtgtggcaaatagtttccaaagccagtctgatgggcaatgggatccccacattgttgagtggcactga

SEQ ID NO: 18 - protein, 346 aa, Homo sapiens
MYNGSCCRIEGDTISQVMPPLLIVAFVLGALGNGVALCGFCFHMKTWKPSTVYLFNLAVADFLLMICLPFRTDYYLRRRHWAFGDIPCRVGLFTLAMNRAGSIVFLTVVAADRYFKVVHPHHAVNTISTRVAAGIVCTLWALVILGTVYLLLENHLCVQETAVSCESFIMESANGWHDIMFQLEFFMPLGIILFCSFKIVWSLRRRQQLARQARMKKATRFIMVVAIVFITCYLPSVSARLYFLWTVPSSACDPSVHGALHITLSFTYMNSMLDPLVYYFSSPSFPKFYNKLKICSLKPKQPGHSKTQRPEEMPISNLGRRSCISVANSFQSQSDGQWDPHIVEWH

SEQ ID NO: 19 - DNA, 1059 bp, Homo sapiens
  CDS (1)..(1059)
atgggccccggcgaggcgctgctggcgggtctcctggtgatggtactggccgtggcgctgctatccaacgcactggtgctgctttgttgcgcctacagcgctgagctccgcactcgagcctcaggcgtcctcctggtgaatctgtctctgggccacctgctgctggcggcgctggacatgcccttcacgctgctcggtgtgatgcgcgggcggacaccgtcggcgcccggcgcatgccaagtcattggcttcctggacaccttcctggcgtccaacgcggcgctgagcgtggcggcgctgagcgcagaccagtggctggcagtgggcttcccactgcgctacgccggacgcctgcgaccgcgctatgccggcctgctgctgggctgtgcctggggacagtcgctggccttctcaggcgctgcacttggctgctcgtggcttggctacagcagcgccttcgcgtcctgttcgctgcgcctgccgcccgagcctgagcgtccgcgcttcgcagccttcaccgccacgctccatgccgtgggcttcgtgctgccgctggcggtgctctgcctcacctcgctccaggtgcaccgggtggcacgcagacactgccagcgcatggacaccgtcaccatgaaggcgctcgcgctgctcgccgacctgcaccccaggtattggcccagtgcatgccgacaggcccaggccagggacttgggcgctccctgggcagttggcttgaggagcctgtgggcatcaccaccgttactctgcccagagttcaccagccacagcactgcccctgcacgctgctcacaggggtttcctgttggttcattggtgcagacactgcgggggcctctgcctcctgggatatgtgctcacagtgcacagggagctttgcgcagagctgtggggtgtgcttctccgggaggggttccgcgggctctgctgtgggcggccagacacacccctcctgtgcatggctgtgggtctgaggcatctgcttgtttctgcccactgctgacccagtgcccttgcatggacttgggcttcaagtcttga

SEQ ID NO: 20 - protein, 352 aa, Homo sapiens [sequence omitted]

SEQ ID NO: 21 - DNA, 984 bp, Homo sapiens
  CDS (1)..(984)
atgacgcccaacagcactggcgaggtgcccagccccattcccaagggggctttggggctctccctggccctggcaagcctcatcatcaccgcgaacctgctcctagccctgggcatcgctgggaccgccgcctgcgcagccacctgctggctgcttcttcctgagcctactgctggctgggctgctcacgggtctggcattgccacattgccagggctgtggaaccagagtcgccggggttactggtcctgcctcctcgtctacttggctcccaacttctccttcctctccctgcttgccaacctcttgctggtgcacggggagcgctacatggcagtcctgaggccactccagccccctgggagcattcggctggccctgctcctcacctgggctggtcccctgctctttgccagtctgcccgctctggggtggaaccactggacccctggtgccaactgcagctcccaggctatcttcccagccccctacctgtacctcgaagtctatgggctcctgctgcccgccgtgggtgctgctgccttcctctctgtccgcgtgctggccactgcccaccgccagctgcaggacatctgccggctggagcgggcagtgtgccgcgatgagccctccgccctggcccgggcccttacctggaggcaggcaagggcacaggctggagccatgctgctcttcgggctgtgctgggggccctacgtggccacactgctcctctcagtcctggcctatgagcagcgcccgccactggggcctgggacactgttgtccctcctctccctaggaagtgccagtgcagcggcagtgcccgtagccatggggctgggcgatcagcgctacacagccccctggaggcagccgcccaaaggtgcctgcaggggctgtggggaagagcctcccgggacagtcccggccccagcattgcctaccacccaagcagccaaagcagtgtcgacctggacttga

SEQ ID NO: 22 - protein, 327 aa, Homo sapiens
MTPNSTGEVPSPIPKGALGLSLALASLIITANLLLALGIAGTAACAATCWLLLPEPTAGWAAHGSGIATLPGLWNQSRRGYWSCLLVYLAPNFSFLSLLANLLLVHGERYMAVLRPLQPPGSIRLALLLTWAGPLLFASLPALGWNHWTPGANCSSQAIFPAPYLYLEVYGLLLPAVGAAAFLSVRVLATAHRQLQDICRLERAVCRDEPSALARALTWRQARAQAGAMLLFGLCWGPYVATLLLSVLAYEQRPPLGPGTLLSLLSLGSASAAAVPVAMGLGDQRYTAPWRQPPKGACRGCGEEPPGTVPAPALPTTQAAKAVSTWT

SEQ ID NO: 23 - DNA, 1044 bp, Homo sapiens
  CDS (1)..(1044)
atgggggatgagctggcaccttgccctgtgggcactacagcttggccggccctgatccagctcatcagcaagacaccctgcatgccccaagcagccagcaacacttccttgggcctgggggacctcagggtgcccagctccatgctgtactggcttttccttccctcaagcctgctggctgcagccacactggctgtcagccccctgctgctggtgaccatcctgcggaaccaacggctgcgacaggagccccactacctgctcccggctaacatcctgctctcagacctggcctacattctcctccacatgctcatctcctccagcagcctgggtggctgggagctgggccgcatggcctgtggcattctcactgatgctgtcttcgccgcctgcaccagcaccatcctgtccttcaccgccattgtgctgcacacctacctggcagtcatccatccactgcgctacctctccttcatgtcccatggggctgcctggaaggcagtggccctcatctggctggtggcctgctgcttccccacattccttatttggctcagcaagtggcaggatgcccagctggaggagcaaggagcttcatacatcctaccaccaagcatgggcacccagccgggatgtggcctcctggtcattgttacctacacctccattctgtgcgttctgttcctctgcacagctctcattgccaactgtttctggaggatctatgcagaggccaagacttcaggcatctgggggcagggctattcccgggccaggggcaccctgctgatccactcagtgctgatcacattgtacgtgagcacaggggtggtgttctccctggacatggtgctgaccaggtaccaccacattgactctgggactcacacatggctcctggcagctaacagtgaggtactcatgatgcttccccgtgccatgctcacatacctgtacctgctccgctaccggcagctgttgggcatggtccggggccacctcccatccaggaggcaccaggccatctttaccatttcctag

SEQ ID NO: 24 - protein, 347 aa, Homo sapiens
MGDELAPCPVGTTAWPALIQLISKTPCMPQAASNTSLGLGDLRVPSSMLYWLFLPSSLLAAATLAVSPLLLVTILRNQRLRQEPHYLLPANILLSDLAYILLHMLISSSSLGGWELGRMACGILTDAVFAACTSTILSFTAIVLHTYLAVIHPLRYLSFMSHGAAWKAVALIWLVACCFPTFLIWLSKWQDAQLEEQGASYILPPSMGTQPGCGLLVIVTYTSILCVLFLCTALIANCFWRIYAEAKTSGIWGQGYSRARGTLLIHSVLITLYVSTGVVFSLDMVLTRYHHIDSGTHTWLLAANSEVLMMLPRAMLTYLYLLRYRQLLGMVRGHLPSRRHQAIFTIS

SEQ ID NO: 25 - DNA, 1002 bp, Homo sapiens
  CDS (1)..(1002)
ggcccccataggagccaacgaagtcatctttgcttcagagctaaaccagtttttcttctctccacagcaaatatcttgacagtgatcatcctctcccagctggtggcaagaagacagaagtcctcctacaactatctcttggcactcgctgctgccgacatcttggtcctctttttcatagtgtttgtggacttcctgttggaagatttcatcttgaacatgcagatgcctcaggtccccgacaagatcatagaagtgctggaattctcatccatccacacctccatatggattactgtaccgttaaccattgacaggtatatcgctgtctgccacccgctcaagtaccacacggtctcatacccagcccgcacccggaaagtcattgtaagtgtttacatcacctgcttcctgaccagcatcccctattactggtggcccaacatctggactgaagactacatcagcacctctgtgcatcacgtcctcatctggatccactgcttcaccgtctacctggtgccctgctccatcttcttcatcttgaactcaatcattgtgtacaagctcaggaggaagagcaattttcgtctccgtggctactccacggggaagaccaccgccatcttgttcaccattacctccatctttgccacactttgggccccccgcatcatcatgattctttaccacctctatggggcgcccatccagaaccgctggctggtacacatcatgtccgacattgccaacatgctagcccttctgaacacagccatcaacttcttcctctactgcttcatcagcaagcggttccgcaccatggcagccgccacgctcaaggctttcttcaagtgccagaagcaacctgtacagttctacaccaatcataacttttccataacaagtagcccctggatctcgccggcaaactcacactgcatcaagatgctggtgtaccagtatgacaaaaatggaaaacctataaaagtatccccgtga

SEQ ID NO: 26 - protein, 333 aa, Homo sapiens
GPHRSQRSHLCFRAKPVFLLSTANILTVIILSQLVARRQKSSYNYLLALAAADILVLFFIVFVDFLLEDFILNMQMPQVPDKIIEVLEFSSIHTSIWITVPLTIDRYIAVCHPLKYHTVSYPARTRKVIVSVYITCFLTSIPYYWWPNIWTEDYISTSVHHVLIWIHCFTVYLVPCSIFFILNSIIVYKLRRKSNFRLRGYSTGKTTAILFTITSIFATLWAPRIIMILYHLYGAPIQNRWLVHIMSDIANMLALLNTAINFFLYCFISKRFRTMAAATLKAFFKCQKQPVQFYTNHNFSITSSPWISPANSHCIKMLVYQYDKNGKPIKVSP

SEQ ID NO: 27 - DNA, 1260 bp, Homo sapiens
  CDS (1)..(1260)
atgctggcagctgcctttgcagactctaactccagcagcatgaatgtgtcctttgctcacctccactttgccggagggtacctgccctctgattcccaggactggagaaccatcatcccggctctcttggtggctgtctgcctggtgggcttcgtgggaaacctgtgtgtgattggcatcctccttcacaatgcttggaaaggaaagccatccatgatccactccctgattctgaatctcagcctggctgatctctccctcctgctgttttctgcacctatccgagctacggcgtactccaaaagtgtttgggatctaggctggtttgtctgcaagtcctctgactggtttatccacacatgcatggcagccaagagcctgacaatcgttgtggtggccaaagtatgcttcatgtatgcaagtgacccagccaagcaagtgagtatccacaactacaccatctggtcagtgctggtggccatctggactgtggctagcctgttacccctgccggaatggttctttagcaccatcaggcatcatgaaggtgtggaaatgtgcctcgtggatgtaccagctgtggctgaagagtttatgtcgatgtttggtaagctctacccactcctggcatttggccttccattattttttgccagcttttatttctggagagcttatgaccaatgtaaaaaacgaggaactaagactcaaaatcttagaaaccagatacgctcaaagcaagtcacagtgatgctgctgagcattgccatcatctctgctctcttgtggctccccgaatgggtagcttggctgtgggtatggcatctgaaggctgcaggcccggccccaccacaaggtttcatagccctgtctcaagtcttgatgttttccatctcttcagcaaatcctctcatttttcttgtgatgtcggaagagttcagggaaggcttgaaaggtgtatggaaatggatgataaccaaaaaacctccaactgtctcagagtctcaggaaacaccagctggcaactcagagggtcttcctgacaaggttccatctccagaatccccagcatccataccagaaaaagagaaacccagctctccctcctctggcaaagggaaaactgagaaggcagagattcccatccttcctgacgtagagcagttttggcatgagagggacacagtcccttctgtacaggacaatgaccctatcccctgggaacatgaagatcaagagacaggggaaggtgttaaatag

SEQ ID NO: 28 - protein, 419 aa, Homo sapiens
MLAAAFADSNSSSMNVSFAHLHFAGGYLPSDSQDWRTIIPALLVAVCLVGFVGNLCVIGILLHNAWKGKPSMIHSLILNLSLADLSLLLFSAPIRATAYSKSVWDLGWFVCKSSDWFIHTCMAAKSLTIVVVAKVCFMYASDPAKQVSIHNYTIWSVLVAIWTVASLLPLPEWFFSTIRHHEGVEMCLVDVPAVAEEFMSMFGKLYPLLAFGLPLFFASFYFWRAYDQCKKRGTKTQNLRNQIRSKQVTVMLLSIAIISALLWLPEWVAWLWVWHLKAAGPAPPQGFIALSQVLMFSISSANPLIFLVMSEEFREGLKGVWKWMITKKPPTVSESQETPAGNSEGLPDKVPSPESPASIPEKEKPSSPSSGKGKTEKAEIPILPDVEQFWHERDTVPSVQDNDPIPWEHEDQETGEGVK

SEQ ID NO: 29 - protein, 13 aa, Artificial Sequence
  peptide
RRLIEDAEYAARG

SEQ ID NO: 30 - DNA, 11 bp, Artificial Sequence
  NF kappa B consensus binding site
ggggactttcc

SEQ ID NO: 31 - DNA, 30 bp, Artificial Sequence
  primer
ccggaattcaccatggatccaaccaccccg

SEQ ID NO: 32 - DNA, 31 bp, Artificial Sequence
  primer
ctagtctagactctacaccagactgcttctc

SEQ ID NO: 33 - DNA, 20 bp, Artificial Sequence
  primer
ttctctgtctacgtcctcag

SEQ ID NO: 34 - DNA, 20 bp, Artificial Sequence
  primer
gtcctgtcatctcttaacag

SEQ ID NO: 35 - protein, 8 aa, Artificial sequence
  Angiopeptin
XCYXKVCT

SEQ ID NO: 36 - protein, 8 aa, Artificial Sequence
  Somatostatin analogue
XCYXKVCX

SEQ ID NO: 37 - DNA, 30 bp, Artificial Sequence
  primer
ccggaattcaccatggagtcctcacccatc

SEQ ID NO: 38 - DNA, 29 bp, Artificial Sequence
  primer
ctagtctagacatcatgactccagccggg

SEQ ID NO: 39 - DNA, 33 bp, Artificial Sequence
  primer
ccggaattcaccatggatccaaccatctcaacc

SEQ ID NO: 40 - DNA, 30 bp, Artificial Sequence
  primer
ctagtctagatcactgctccaatctgcttc

SEQ ID NO: 41 - DNA, 33 bp, Artificial Sequence
  primer
cagagaattcgttatgctgtccattttgcttcc

SEQ ID NO: 42 - DNA, 33 bp, Artificial Sequence
  primer
tacttctagacccaccagcactcatctgtgtac

SEQ ID NO: 43 - DNA, 38 bp, Artificial Sequence
  primer
ccggaattcaccatgaaccagactttgaatagcagtgg

SEQ ID NO: 44 - DNA, 37 bp, Artificial Sequence
  primer
ctagtctagatctcaagcccccatctcattggtgccc

SEQ ID NO: 45 - DNA, 28 bp, Artificial Sequence
  primer
ccggaattcaccatggaagctgacctgg

SEQ ID NO: 46 - DNA, 29 bp, Artificial Sequence
  primer
ctagtctagactcacgtggggcctgcgcc

SEQ ID NO: 47 - DNA, 35 bp, Artificial Sequence
  primer
cagagaattcctgcaattctattctagctcctgtg

SEQ ID NO: 48 - DNA, 33 bp, Artificial Sequence
  primer
gcgggatcctattgtcaaccaagctgtgacatg

SEQ ID NO: 49 - DNA, 27 bp, Artificial Sequence
  primer
ccggaattcgccatgtacaacgggtcg

SEQ ID NO: 50 - DNA, 30 bp, Artificial Sequence
  primer
ctagtctagattcagtgccactcaacaatg

(19) United States
(12) Patent Application Publication
(10) Pub. No.: US 2003/0108986 A1
(43) Pub. Date: Jun. 12, 2003

(54) Compositions and methods comprising G-protein coupled receptors

(75) Inventors: Didier Communi, Dilbeek (BE); Vincent Lannoy, Brussels (BE); Stephane Brezillon, Brussels (BE); Michel Detheux, Mons (BE); Marc Parmentier, Brussels (BE); Cedric Govaerts, Brussels (BE)

Correspondence Address: PALMER & DODGE, LLP, KATHLEEN M. WILLIAMS, 111 HUNTINGTON AVENUE, BOSTON, MA, 02199, US

(73) Assignee: Euroscreen, S.A.

(21) Appl. No.: 10/079,384

(22) Filed: Feb. 20, 2002

Related U.S. Application Data
(63) Continuation-in-part of application No. 09/885,453, filed on Jun. 21, 2001.

(51) Int. Cl. G01N 33/53; A01K 67/00; C07H 21/04; C12P 21/02; C12N 5/06; C07K 14/705

(52) U.S. Cl. 435/69.1; 435/320.1; 435/325; 530/350; 536/23.5; 800/8; 435/7.1

(57) ABSTRACT

The present invention relates to G-protein coupled receptors and the nucleic acid molecules encoding them. The invention further relates to methods of screening for compounds which modulate the activity of one or more of the G-protein coupled receptors disclosed herein, and methods for modulating receptor activity. The invention also provides a natural ligand for one or more of the G-protein coupled receptors disclosed herein, and methods for identifying other natural ligands for these receptors.

### RELATED APPLICATION

This application claims priority under 35 U.S.C. §120 to U.S. application Ser. No. 09/885,453, filed Jun. 20, 2001, which is incorporated herein by reference in its entirety.

### BACKGROUND OF THE INVENTION

G-protein coupled receptors (GPCRs) are proteins responsible for transducing a signal within a cell. GPCRs have usually seven transmembrane domains. Upon binding of a ligand to an extra-cellular portion or fragment of a GPCR, a signal is transduced within the cell that results in a change in a biological or physiological property or behavior of the cell. GPCRs, along with G-proteins and effectors (intracellular enzymes and channels modulated by G-proteins), are the components of a modular signaling system that connects the state of intra-cellular second messengers to extra-cellular inputs.

GPCR genes and gene products can modulate various physiological processes and are potential causative agents of disease. The GPCRs seem to be of critical importance to both the central nervous system and peripheral physiological processes.

The GPCR protein superfamily is represented in five families: Family I, receptors typified by rhodopsin and the beta2-adrenergic receptor and currently represented by over 200 unique members; Family II, the parathyroid hormone/calcitonin/secretin receptor family; Family III, the metabotropic glutamate receptor family, Family IV, the CAMP receptor family, important in the chemotaxis and development of D. discoideum; and Family V, the fungal mating pheromone receptor such as STE2.

G proteins represent a family of heterotrimeric proteins composed of α, β and γ subunits, that bind guanine nucleotides. These proteins are usually linked to cell surface receptors (receptors containing seven transmembrane domains) for signal transduction. Indeed, following ligand binding to the GPCR, a conformational change is transmitted to the G protein, which causes the α-subunit to exchange a bound GDP molecule for a GTP molecule and to dissociate from the βγ-subunits.

The GTP-bound form of the α, β and γ-subunits typically functions as an effector-modulating moiety, leading to the production of second messengers, such as cAMP (e.g. by activation of adenyl cyclase), diacylglycerol or inositol phosphates.

Greater than 20 different types of α-subunits are known in humans. These subunits associate with a small pool of β and γ subunits. Examples of mammalian G proteins include Gi, Go, Gq, Gs and Gt. G proteins are described extensively in Lodish et al., Molecular Cell Biology (Scientific American Books Inc., New York, N.Y., 1995; and also by Downes and Gautam, 1999, The G-Protein Subunit Gene Families. Genomics 62:544-552), the contents of both of which are incorporated herein by reference.

Known and uncharacterized GPCRs currently constitute major targets for drug action and development. There are ongoing efforts to identify new G protein coupled receptors which can be used to screen for new agonists and antagonists having potential prophylactic and therapeutic properties.

More than 300 GPCRs have been cloned to date, excluding the family of olfactory receptors. Mechanistically, approximately 50-60% of all clinically relevant drugs act by modulating the functions of various GPCRs (Cudermann et al., J. Mol. Med., 73:51-63, 1995).

### SUMMARY OF THE INVENTION

The present invention is related to newly identified members of the G-protein coupled receptor superfamily. The present invention is based in part on the discovery that angiopeptin is a natural ligand for one of these receptors: GPCRx11. This invention therefore relates, in part, to the angiopeptin/receptor pair, and to functional homologs of the receptor which also bind angiopeptin and cells transformed by a vector comprising the nucleotide sequence (SEQ ID NO; 11) encoding the GPCRx11 receptor amino acid sequence (SEQ ID NO: 12). The invention further relates to a composition comprising an isolated GPCRx11 polypeptide and angiopeptin, as well as to methods of identifying agents that modulate the activities of GPCRx11 polypeptides. The invention still further relates to methods of identifying molecules which bind to the newly discovered G-protein coupled receptors described herein, and methods of identifying a ligand for these receptors. The methods described herein are useful for the identification of agonists, inverse agonist, or antagonist compounds useful for the development of new drugs. The interaction of GPCRx11 with angiopeptin is also useful for the development of diagnostics for diseases related to GPCRx11 activity.

The present invention provides an isolated G-protein coupled receptor comprising an amino acid sequence which is at least 90% identical to a sequence selected from the group consisting of SEQ ID Nos. 2, 4, 6, 8, 10, 12, 14, 16, 18, 20, 22, 24, 26, and 28.

The invention further provides an isolated G-protein coupled receptor comprising an amino acid sequence selected from the group consisting of SEQ ID Nos. 2, 4, 6, 8, 10, 12, 14, 16, 18, 20, 22, 24, 26, and 28.

The invention still further provides an isolated polynucleotide encoding a G-protein coupled receptor as described herein.

In one embodiment, the isolated polynucleotide the polynucleotide encoding each of said G-protein coupled receptors of SEQ ID Nos. 2, 4, 6, 8, 10, 12, 14, 16, 18, 20, 22, 24, 26, and 28, comprises the sequence of SEQ ID Nos. 1, 3, 5, 7, 9, 11, 13, 15, 17, 19, 21, 23, 25, and 27, respectively.

The invention provides a nucleic acid vector comprising a polynucleotide sequence selected from the group consisting of SEQ ID Nos. 1, 3, 5, 7, 9, 11, 13, 15, 17, 19, 21, 23, 25, and 27.

The invention also provides a cell comprising the nucleic acid vector comprising a polynucleotide sequence selected from the group consisting of SEQ ID Nos. 1, 3, 5, 7, 9, 11, 13, 15, 17, 19, 21, 23, 25, and 27.

In one embodiment, the polynucleotide sequence of said expression vector is expressed in the cell membrane of said cell.

The present invention further provides a non-human mammal having a homozygous null mutation in the gene comprising a polynucleotide selected from the group consisting of SEQ ID Nos. 2, 4, 6, 8, 10, 12, 14, 16, 18, 20, 22, 24, 26, and 28.

The invention further provides a non-human mammal transgenic for a polynucleotide encoding a G-protein coupled receptor, wherein said polynucleotide is selected from the group consisting of SEQ ID Nos. 1, 3, 5, 7, 9, 11, 13, 15, 17, 19, 21, 23, 25, and 27.

The present invention encompasses a method of identifying an agent that modulates the function of GPCRx11, said method comprising: (a) contacting a GPCRx11 polypeptide with angiopeptin in the presence and absence of a candidate modulator under conditions permitting the binding of said angiopeptin to said GPCRx11 polypeptide; and (b) measuring the binding of said GPCRx11 polypeptide to said angiopeptin, wherein a decrease in binding in the presence of said candidate modulator, relative to the binding in the absence of said candidate modulator, identifies said candidate modulator as an agent that modulates the function of GPCRx11.

The invention also encompasses a method of detecting, in a sample, the presence of an agent that modulates the function of GPCRx11, said method comprising: (a) contacting a GPCRx11 polypeptide with angiopeptin in the presence and absence of said sample under conditions permitting the binding of said angiopeptin to said GPCRxI polypeptide; and (b) measuring the binding of said GPCRx11 polypeptide to said angiopeptin, wherein a decrease in binding in the presence of said sample, relative to the binding in the absence of said sample, indicates the presence, in said sample of an agent that modulates the function of GPCRx11.

The invention further encompasses A method of identifying an agent that modulates the function of GPCRx11, said method comprising: (a) contacting a GPCRx11 polypeptide with angiopeptin in the presence and absence of a candidate modulator; and (b) measuring a signaling activity of said GPCRx11 polypeptide, wherein a change in the activity in the presence of said candidate modulator relative to the activity in the absence of said candidate modulator identifies said candidate modulator as an agent that modulates the function of GPCRx11.

The invention still further encompasses a method of identifying an agent that modulates the function of GPCRx11, said method comprising: (a) contacting a GPCRx11 polypeptide with a candidate modulator; (b) measuring a signaling activity of said GPCRx11 polypeptide in the presence of said candidate modulator; and (c) comparing said activity measured in the presence of said candidate modulator to said activity measured in a sample in which said GPCRx11 polypeptide is contacted with angiopeptin at its EC₅₀, wherein said candidate modulator is identified as an agent that modulates the function of GPCRx11 when the amount of said activity measured in the presence of said candidate modulator is at least 20% of the amount induced by said angiopeptin present at its EC₅₀.

The invention encompasses a method of detecting the presence, in a sample, of an agent that modulates the function of GPCRx11, said method comprising: (a) contacting a GPCRx11 polypeptide with angiopeptin in the presence and absence of said sample; (b) measuring a signaling activity of said GPCRx11 polypeptide; and (c) comparing the amount of said activity measured in a reaction containing GPCRx11 and angiopeptin without said sample to the amount of said activity measured in a reaction containing GPCRx11, angiopeptin and said sample, wherein a change in said activity in the presence of said sample relative to the activity in the absence of said sample indicates the presence, in said sample, of an agent that modulates the function of GPCRx11.

The invention further encompasses a method of detecting the presence, in a sample, of an agent that modulates the function of GPCRx11, said method comprising: (a) contacting a GPCRx11 polypeptide with said sample; (b) measuring a signaling activity of said GPCRx11 polypeptide in the presence of said sample; and (c) comparing said activity measured in the presence of said sample to said activity measured in a reaction in which said GPCRx11 polypeptide is contacted with angiopeptin present at its EC₅₀, wherein an agent that modulates the function of GPCRx11 is detected if the amount of said activity measured in the presence of said sample is at least 20% of the amount induced by said angiopeptin present at its EC₅₀.

In one embodiment of any of the preceding methods, angiopeptin is detectably labeled.

In one embodiment, angiopeptin is detectably labeled with a moiety selected from the group consisting of a radioisotope, a fluorophore, a quencher of fluorescence, an enzyme, and an affinity tag.

In one embodiment of any of the preceding methods, the step of contacting is performed in or on a cell expressing said GPCRx11 polypeptide.

In one embodiment of any of the preceding methods, the step of contacting is performed in or on synthetic liposomes.

In one embodiment of any of the preceding methods, the step of contacting is performed in or on virus-induced budding membranes containing a GPCRx11 polypeptide.

In one embodiment of any of the preceding methods, the method is performed using a membrane fraction from cells expressing said GPCRx11 polypeptide.

In one embodiment of any of the preceding methods, the step of measuring binding is performed using a method selected from label displacement, surface plasmon resonance, fluorescence resonance energy transfer, fluorescence quenching, and fluorescence polarization.

In one embodiment of any of the preceding methods, the agent is selected from the group consisting of a peptide, a polypeptide, an antibody or antigen-binding fragment thereof, a lipid, a carbohydrate, a nucleic acid, and a small organic molecule.

In one embodiment of any of the preceding methods, the step of measuring a signaling activity of said GPCRx11 polypeptide comprises detecting a change in the level of a second messenger.

In one embodiment of any of the preceding methods, the step of measuring a signaling activity comprises measurement of guanine nucleotide binding or exchange, adenylate cyclase activity, cAMP, Protein Kinase C activity, phosphatidylinosotol breakdown, diacylglycerol, inositol triphosphate, intracellular calcium, arachidoinc acid, MAP kinase activity, tyrosine kinase activity, or reporter gene expression.

In one embodiment of any of the preceding methods, the step of measuring a signaling activity comprises using an aequorin-based assay.

The present invention encompasses a method of identifying a molecule which binds to a G-protein coupled receptor comprising: (a) contacting a G-protein coupled receptor having an amino acid sequence selected from the group consisting of SEQ ID Nos. 2, 4, 6, 8, 10, 12, 14, 16, 18, 20, 22, 24, 26, and 28, with a candidate molecule; (b) measuring the binding of said G-protein coupled receptor to said candidate molecule, wherein a detectable level of binding indicates that said candidate molecule binds to said G-protein coupled receptor.

In one embodiment, the candidate molecule is detectably labeled.

In a further embodiment, the candidate molecule is detectably labeled with a moiety selected from the group consisting of a radioisotope, a fluorophore, a quencher of fluorescence, an enzyme, and an affinity tag.

In one embodiment of the preceding method, the step of measuring is performed using a method selected from label displacement, surface plasmon resonance, fluorescence resonance energy transfer, fluorescence quenching, and fluorescence polarization.

The invention also encompasses a method of identifying a ligand for a G-protein coupled receptor comprising: (a) contacting a G-protein coupled receptor having an amino acid sequence selected from the group consisting of SEQ ID Nos. 2, 4, 6, 8, 10, 12, 14, 16, 18, 20, 22, 24, 26, and 28, with a candidate ligand; (b) measuring a signaling activity of said G-protein coupled receptor in the presence of said candidate ligand, wherein a ligand for said G-protein coupled receptor is identified if an increase in said signaling activity is measured in the presence of said candidate ligand relative to said signaling activity in the absence of said candidate ligand.

In one embodiment of the preceding method, the candidate ligand is detectably labeled.

In one embodiment of the two preceding methods, the step of contacting is performed in or on a cell expressing said G-protein coupled receptor.

In one embodiment of the two preceding methods, the step of contacting is performed in or on synthetic liposomes.

In one embodiment of the two preceding methods, the step of contacting is performed in or on virus-induced budding membranes containing said G-protein coupled receptor.

In one embodiment of the two preceding methods, the method is performed using a membrane fraction from cells expressing said G-protein coupled receptor.

In one embodiment of the above methods, the step of measuring a signaling activity of said G-protein coupled receptor comprises detecting a change in the level of a second messenger.

In a further embodiment of the above methods, the step of measuring a signaling activity comprises measurement of guanine nucleotide binding or exchange, adenylate cyclase activity, cAMP, Protein Kinase C activity, phosphatidylinosotol breakdown, diacylglycerol, inositol triphosphate, intracellular calcium, arachinoid acid, MAP kinase activity, tyrosine kinase activity, or reporter gene expression.

In a still further embodiment, the step of measuring a signaling activity comprises using an aequorin-based assay.

The invention encompasses a method of modulating the activity of a GPCRx11 polypeptide in a cell, said method comprising the step of delivering to said cell an agent that modulates the activity of a GPCRx11 polypeptide, such that the activity of GPCRx11 is modulated.

The invention further encompasses a method of diagnosing a disease or disorder characterized by dysregulation of GPCRx11 signaling, said method comprising: (a) isolating nucleic acid from a tissue sample; (b) amplifying a GPCRx11 polynucleotide, using said nucleic acid as a template; and (c) comparing the amount of amplified GPCRx11 polynucleotide produced in step (b) with a standard, wherein a difference in said amount of amplified GPCRx11 polynucleotide relative to said standard is diagnostic of a disease or disorder characterized by dysregulation of GPCRx11.

The present invention further encompasses a composition consisting essentially of an isolated GPCRx11 polypeptide and isolated angiopeptin.

The invention encompasses a kit for screening for agents that modulate the activity of GPCRx11, said kit comprising an isolated GPCRx11 polypeptide and isolated angiopeptin.

The invention also encompasses a kit for screening for agents that modulate the activity of GPCRx11, said kit comprising isolated angiopeptin and a cell membrane fraction comprising a GPCRx11 polypeptide.

The invention further encompasses a kit for screening for agents that modulate the activity of GPCRx11, said kit comprising an isolated polynucleotide encoding a GPCRx11 polypeptide and isolated angiopeptin.

The invention still further encompasses a kit for screening for agents that modulate the activity of GPCRx11, said kit comprising a cell transformed with a polynucleotide encoding a GPCRx11 polypeptide and angiopeptin.

The invention also encompasses a kit for the diagnosis of a disease or disorder characterized by dysregulation of GPCRx11 signaling, said kit comprising an isolated polynucleotide encoding a GPCRx11 polypeptide, a standard and packaging materials therefor.

The invention also encompasses a kit for the diagnosis of a disease or disorder characterized by dysregulation of GPCRx11 signaling, said kit comprising a cellular membrane fraction comprising a GPCRx11 polypeptide, a standard and packaging materials therefor.

The invention also encompasses a kit for the diagnosis of a disease or disorder characterized by dysregulation of GPCRx11 signaling, said kit comprising a cell transformed with a polynucleotide encoding a GPCRx11 polypeptide, a standard and packaging materials therefor.

In one embodiment, either of the three preceding kits further comprise angiopeptin.

In one embodiment of the kits described above, the standard comprises a sample from an individual not affected by said disease or disorder.

As used herein, the term “GPCRx11 polypeptide” refers to a polypeptide having two essential properties: 1) a GPCRx11 polypeptide has at least 80% amino acid identity, preferably 85%, 90%, 95%, or higher, up to and including 100% identity, with SEQ ID NO. 11; and 2) a GPCRx11 polypeptide has GPCRx11 activity including either or both of GPCRx11 ligand binding activity or GPCRx11 signaling activity as defined herein.

As used herein, “GPCRx11 activity” refers to angiopeptin binding to or signaling by a GPCRx11 polypeptide as defined herein. A polypeptide that has “GPCRx11 activity” will bind to angiopeptin and stimulate mitochondrial aequorin fluorescence with an EC₅₀ of no greater than 160 nM.

A “homologous sequence” (which may exist in other mammal species or specific groups of human populations), where homology indicates sequence identity, means a sequence which presents a high sequence identity (more than 80%, 85%, 90%, 95% or 98% sequence identity) with the nucleotide sequences of SEQ ID Nos.: 1, 3, 5, 7, 9, 11, 13, 15, 17, 19, 21, 23, 25, or 27 or amino acid sequence of SEQ ID Nos.: 2, 4, 6, 8, 10, 12, 14, 16, 18, 20, 22, 24, 26, or 27. A functional homolog, or GPCRx11 for example, is characterized by the ability to bind angiopeptin ligand as defined herein or by the ability to initiate or propagate a signal in response to ligand binding, or both.

Homologous sequences of a sequence according to the invention may include an amino acid or nucleotide sequence encoding a similar receptor which exists in other animal species (rat, mouse, cat, dog, etc.) or in specific human population groups, but which are involved in the same biochemical pathway(s).

Such homologous sequences may comprise additions, deletions or substitutions of one or more amino acids or nucleotides, which do not substantially alter the functional characteristics of the receptor according to the invention. For example, homologs of GPCRx11 will have at least 90% of the activity of wt full length human GPCRx11 and will bind angiopeptin.

Such homologous sequences can also be nucleotide sequences of more than 400, 600, 800 or 1000 nucleotides which are able to hybridize to the complete nucleic acid sequences of SEQ ID Nos.: 1, 3, 5, 7, 9, 11, 13, 15, 17, 19, 21, 23, or 25 under stringent hybridization conditions (such as the ones described by SAMBROOK et al., Molecular Cloning, Laboratory Manuel, Cold Spring, Harbor Laboratory press, New York). An example of “stringent hybridization conditions” is as follows: hybridize in 50% formamide, 5×SSC, 50 mM sodium phosphate (pH 6.8), 0.1% sodium pyrophosphate, 5×Denhardt's solution, 50 μg/ml sonicated salmon sperm DNA, 0.1% SDS and 10% dextran sulfate at 42° C.; and wash at 42° C. (or higher, e.g., up to two degrees C. below the Tₘ of the perfect complement of the probe sequence) in 0.2×SSC and 0.1% SDS.

As used herein, the term “GPCRx11 signaling activity” refers to the initiation or propagation of signaling by a GPCRx11 polypeptide. GPCRx11 signaling activity is monitored by measuring a detectable step in a signaling cascade by assaying one or more of the following: stimulation of GDP for GTP exchange on a G protein; alteration of adenylate cyclase activity; protein kinase C modulation; phosphatidylinositol breakdown (generating second messengers diacylglycerol, and inositol triphosphate); intracellular calcium flux; activation of MAP kinases; modulation of tyrosine kinases; or modulation of gene or reporter gene activity. A detectable step in a signaling cascade is considered initiated or mediated if the measurable activity is altered by 10% or more above or below a baseline established in the substantial absence of angiopeptin relative to any of the GPCRx11 activity assays described herein below. The measurable activity can be measured directly, as in, for example, measurement of cAMP or diacylglycerol levels. Alternatively, the measurable activity can be measured indirectly, as in, for example, a reporter gene assay.

As used herein, the term “detectable step” refers to a step that can be measured, either directly, e.g., by measurement of a second messenger or detection of a modified (e.g., phosphorylated) protein, or indirectly, e.g., by monitoring a downstream effect of that step. For example, adenylate cyclase activation results in the generation of cAMP. The activity of adenylate cyclase can be measured directly, e.g., by an assay that monitors the production of cAMP in the assay, or indirectly, by measurement of actual levels of cAMP.

A “cell” as used herein can be either a prokaryotic or eukaryotic cell which is capable of expressing a nucleic acid molecule as described herein. Preferably the cell is a recombinant cell. Preferably, a recombinant cell according to the invention is a recombinant cell transformed by a plasmid, cosmid or viral vector, preferably a baculovirus, an adenovirus, or a semliki forest virus, and the cell is preferably selected from the group consisting of bacterial cells, yeast cells, insect cells or mammal cells.

According to a preferred embodiment of the present invention, the cell is selected from the group consisting of COS-7 cells, a CHO cell, a LM (TK-) cell, a NIH-3T3 cell, HEK-293 cell, K-562 cell or a 1321N1 astrocytoma cell. Other transfectable cell lines are also useful, however. Preferably, the vector comprises regulatory elements operatively linked to the polynucleotide sequence encoding the receptor according to the invention, so as to permit expression thereof.

Another aspect of the present invention is related to the use of a specific active portion of the sequences. As used herein, an “active portion” refers to a portion of a sequence that is of sufficient size to exhibit normal or near normal pharmacology (e.g., receptor activity (as defined herein), the response to an activator or inhibitor, or ligand binding are at least 90% of the level of activity, response, or binding exhibited by a wild type receptor). “A portion” as it refers to a sequence encoding a receptor, refers to less than 100% of the sequence (i.e., 99, 90, 80, 70, 60, 50% etc . . . ). The active portion could be a receptor which comprises a partial deletion of the complete nucleotide or amino acid sequence and which still maintains the active site(s) and protein domain(s) necessary for the binding of and interaction with a specific ligand, preferably acetate and propionate.

In another embodiment of any of the preceding methods, the contacting is performed in or on synthetic liposomes (Mirzabekov et al., (2000) Nat Biotechnol. 18: 649-54) or virus-induced budding membranes containing a GPCRx11 polypeptide. (see Patent application WO0102551, Virus-like particles, their Preparation and their Use preferably in Pharmaceutical Screening and Functional Genomics (2001) incorporated herein by reference).

As used herein, “ligand” refers to a moiety that is capable of associating or binding to a receptor. According to the method of the invention, a ligand and a receptor have a binding constant that is sufficiently strong to allow detection of binding by an assay method that is appropriate for detection of a ligand binding to a receptor (e.g. a second messenger assay to detect an increase or decrease in the production of a second messenger in response to ligand binding to the receptor, a binding assay to measure protein-ligand binding or an immunoassay to measure antibody-antigen interactions). A ligand according to the invention includes the actual molecule that binds a receptor (e.g. angiopeptin is the ligand for GPCRx11) or a ligand may be any nucleotide, antibody, antigen, enzyme, peptide, polypeptide or nucleic acid capable of binding to the receptor. According to the method of the invention, a ligand and receptor specifically bind to each other (e.g. via covalent or hydrogen bonding or via an interaction between, for example, a protein and a ligand, an antibody and an antigen or protein subunits).

Another aspect of the present invention is related to a method for the screening, detection and recovery of candidate modulators of a receptor of the invention comprising the steps of: contacting a cell expressing GPCRx11 with angiopeptin under conditions which permit binding of angiopeptin to GPCRx11, in the presence of the candidate modulator, performing a second messenger assay, and comparing the results of the second messenger assay obtained in the presence and absence of the candidate modulator.

Another aspect of the present invention is related to a method for the screening, detection and possible recovery of candidate modulators of a receptor of the invention comprising the steps of: contacting a cell membrane expressing GPCRx11 with angiopeptin under conditions which permit binding of angiopeptin to GPCRx11, performing a second messenger assay, and comparing the results of the second messenger assay obtained in the presence and absence of the candidate modulator.

In another embodiment, the step of measuring a signaling activity of the GPCRx11 polypeptide comprises detecting a change in the level of a second messenger.

A fuirther aspect of the present invention is related to the unknown agonist and/or antagonist compounds identified and/or recovered by the method of the invention, as well as to a diagnostic kit comprising the (unknown) compounds or a pharmaceutical composition (including a vaccine) comprising an adequate pharmaceutical carrier and a sufficient amount of the (unknown) compound.

An antagonist compound according to the invention means a molecule or a group of molecules able to bind to the receptor according to the invention and block the binding of natural compounds (angiopeptin).

The invention further encompasses a method of diagnosing a disease or disorder characterized by dysregulation of GPCRx11 signaling, the method comprising: a) contacting a tissue sample with an antibody specific for a GPCRx11 polypeptide and an antibody specific for a GPCRx11 ligand; b) detecting binding of the antibodies to the tissue sample; and c) comparing the binding detected in step (b) with a standard, wherein a difference in binding of either antibody or both, relative to the standard, is diagnostic of a disease or disorder characterized by dysregulation of GPCRx11

The invention further encompasses a method of diagnosing a disease or disorder characterized by dysregulation of GPCRx11 signaling, the method comprising: a) isolating a tissue sample; b) measuring the concentration of angiopeptin; and c) comparing the amount of angiopeptin measured in step (b) with a standard, wherein a difference in the amount of angiopeptin relative to the standard is diagnostic of a disease or disorder characterized by dysregulation of GPCRx11.

A further aspect of the present invention is related to a non-human mammal comprising a homozygous null mutation (homozygous “knock-out”) of the polynucleotide sequence encoding the GPCRx11 receptor according to the invention, or a transgenic non-human mammal that over expresses a GPCRx11 polypeptide above the natural level of expression. As used herein. “above the natural level of expression” refers to a level that is at least 2-fold, preferably 5-fold, more preferably 10-fold and most preferably 100-fold or more (i.e., 150-fold, 200-fold, 250-fold, 500-fold, 1000-fold, 10,000-fold etc.) as compared to the level of expression of the endogenous receptor in its normal native context. A transgenic non-human mammal according to the invention will express the transgene in at least one tissue or cell type but can express the GPCRx11 transgene in all tissues and cells. A transgenic non-human mammal can be obtained by a method well known by a person skilled in the art, for instance, as described in document WO 98/20112 using the classical technique based upon the transfection of embryonic stem cells, preferably according to the method described by Carmeliet et al. (Nature, Vol.380, p.435-439, 1996).

“Gene targeting” is a type of homologous recombination that occurs when a fragment of genomic DNA is introduced into a mammalian cell and that fragment locates and recombines with endogenous homologous sequences as exemplified in U.S. Pat. Nos. 5,464,764, and 5,777,195, the contents of which are hereby incorporated by reference herein in their entireties. As used herein the term “transgenic animal” refers to a non-human animal in which one or more, and preferably essentially all, of the cells of the animal contain a transgene introduced by way of human intervention, such as by transgenic techniques known in the art. The transgene can be introduced into the cell, directly or indirectly by introduction into a precursor of the cell, by way of deliberate genetic manipulation, such as by microinjection or by infection with a recombinant virus.

Preferably, the transgenic non-human mammal overexpressing the polynucleotide encoding the GPCRx11 receptor according to the invention comprises the polynucleotide incorporated in a DNA construct with an inducible promoter allowing the overexpression of the receptor and possibly also tissue and cell-specific regulatory elements.

In one embodiment, the kits according to the invention comprise reagents for measuring the binding of angiopeptin to a GPCRx11 polypeptide. In another embodiment, the kit comprises reagents for measuring a signaling activity of a GPCRx11 polypeptide.

In one embodiment, a screening or diagnostic kit according to the invention includes a GPCRx11 receptor polypeptide or a cellular membrane preparation comprising a GPCRx11 polypeptide and angiopeptin in separate containers. Such kits can additionally comprise all the necessary means and media for performing a detection of specific binding (for example of propionate) to the GPCRx11 receptor according to the invention. Binding or signaling activity can be correlated with a method of monitoring one or more of the symptoms of the diseases described hereafter.

The diagnostic kits can thus further comprise elements necessary for a specific diagnostic measurement, or, for example, the measurements of bound compounds using high throughput screening techniques known to the person skilled in the art, e.g., the techniques described in WO 00/02045. Such kits can be used, e.g. to monitor dosage and effectiveness of GPCRx11 modulating agents used for treatment. The high throughput screening diagnostic dosage and monitoring can be performed by using various solid supports, such as microtiter plates or biochips selected by the person skilled in the art.

In a pharmaceutical composition according to the invention, the adequate pharmaceutical carrier is a carrier of solid, liquid or gaseous form, which can be selected by the person skilled in the art according to the type of administration and the possible side effects of the compound administered to modulate GPCRx11 activity. The pharmaceutical carrier useful according to the invention does not include tissue culture medium or other media comprising serum. The ratio between the pharmaceutical carrier and the specific compound can be selected by the person skilled in the art according to the patient treated, the administration and the possible side effects of the compound, as well as the type of disease of disorder treated or sought to be prevented.

The pharmaceutical composition finds advantageous applications in the field of treatment and/or prevention of various diseases or disorders, preferably selected from the group consisting of viral infections or diseases induced by various viruses or bacteria, disturbances of cell migration, diseases or perturbations of the immune system, including cancer, development of tumours and tumour metastasis, inflammatory and neo-plastic processes, bacterial and fungal infections, for wound and bone healing and dysfunction of regulatory growth functions, pains, diabetes, obesity, anorexia, bulimia, acute heart failure, hypotension, hypertension, urinary retention, osteoporosis, angina pectoris, myocardial infarction, restenosis, atherosclerosis, diseases characterised by excessive smooth muscle cell proliferation, aneurysms, wound healing, diseases characterised by loss of smooth muscle cells or reduced smooth muscle cell proliferation, stroke, ischemia, ulcers, allergies, benign prostatic hypertrophy, migraine, vomiting, psychotic and neurological disorders, including anxiety, schizophrenia, maniac depression, depression, delirium, dementia and severe mental retardation, degenerative diseases, neurodegenerative diseases such as Alzheimer's disease or Parkinson's disease, and dyskinasias, such as Huntington's disease or Gilles de la Tourett's syndrome and other related diseases

Among the mentioned diseases the preferred applications are related to therapeutic agents targeting 7TM receptors that can play a function in preventing, improving or correcting dysfunctions or diseases, including, but not limited to fertility, fetal development, infections such as bacterial, fungal, protozoan and viral infections, particularly infections caused by HIV1 and HIV2, pain, cancer, anorexia, bulimia, asthma, Parkinson's disease, acute heart failure, hypertension, urinary retention, osteoporosis, angina pectoris, myocardial infarction, ulcers, asthma, allergies, benign prostatic hypertrophy, psychotic and neurological disorders including anxiety, depression, migraine, vomiting, stroke, schizophrenia, manic depression, delirium, dementia, severe mental retardation and dyskinesias, such as Huntington's disease or Gilles de la Tourette's syndrome including thrombosis and other cardiovascular diseases, autoimmune and inflammatory diseases.

As used herein, an “antagonist” is a ligand which competitively binds to a receptor at the same site as an agonist, but does not activate an intracellular response initiated by an active form of the receptor. An antagonist thereby inhibits the intracellular response induced by an agonist, for example angiopeptin, by at least 10%, preferably 15-25%, more preferably 25-50% and most preferably, 50-100%, as compared to the intracellular response in the presence of an agonist and in the absence of an antagonist.

As used herein, an “agonist” refers to a ligand that activates an intracellular response when it binds to a receptor at concentrations equal to or lower than angiopeptin concentrations which induce an intracellular response. An agonist according to the invention can increase the intracellular response mediated by a receptor by at least 2-fold, preferably 5-fold, more preferably 10-fold and most preferably 100-fold or more (i.e., 150-fold, 200-fold, 250-fold, 500-fold, 1000-fold, 10,000-fold etc . . . ), as compared to the intracellular response in the absence of agonist. An agonist according to the invention may decrease internalization of a cell surface receptor such that the cell surface expression of a receptor is increased by at least 2-fold, preferably 5-fold, more preferably 10-fold and most preferably, 100-fold or more (i.e., 150-fold, 200-fold, 250-fold, 500-fold, 1000-fold, 10,000-fold etc . . . ), as compared to the number of cell surface receptors present on the surface of a cell in the absence of an agonist. In another embodiment of the invention, an agonist stabilizes a cell surface receptor and increases the cell surface expression of a receptor by at least 2-fold, preferably 5-fold, more preferably 10-fold and most preferably, 100-fold or more (i.e., 200-fold, 250-fold, 500-fold, 1000-fold, 10,000-fold etc . . . ), as compared to the number of cell surface receptors present on the surface of a cell in the absence of agonist.

As used herein, an “inverse agonist” refers to a ligand which decreases a constitutive activity of a cell surface receptor when it binds to a receptor. An inverse agonist according to the invention can decrease the constitutive intracellular response mediated by a receptor by at least 2-fold, preferably 5-fold, more preferably 10-fold and most preferably 100-fold or more (i.e., 150-fold, 200-fold, 250-fold, 500-fold, 1000-fold, 10,000-fold etc . . . ), as compared to the intracellular response in the absence of inverse agonist.

An “inhibitor” compound according to the invention is a molecule directed against the receptor or against the natural ligand for the receptor that decreases the binding of the ligand to the receptor by at least 10%, preferably 15-25%, more preferably 25-50% and most preferably, 50-100%, in the presence of, for example, angiopeptin, as compared to the binding in the presence of angiopeptin and in the absence of inhibitor. An “inhibitor” compound of the invention can decrease the intracellular response induced by an agonist by at least 10%, preferably 15-25%, more preferably 25-50% and most preferably, 50-100%. An “inhibitor” also refers to a nucleotide sequence encoding an inhibitor compound of the invention.

As used herein, “natural ligand” refers to a naturally occurring ligand, found in nature, which binds to a G-protein coupled receptor. A “natural ligand” does not refer to an engineered ligand that is not found in nature and that is engineered to bind to a receptor, where it did not formerly do so in a manner different, either in degree or kind, from that which it was engineered to do. Such an engineered ligand is no longer naturally-occurring but is “non-natural” even if it is derived from a naturally occurring molecule.

As used herein, a “modulator” refers to a compound that increases or decreases the cell surface expression of a receptor of the invention, increases or decreases the binding of a ligand to a receptor of the invention, or any compound that increases or decreases the intracellular response initiated by an active form of the receptor of the invention, either in the presence or absence of an agonist, and in the presence of a ligand for the receptor, for example angiopeptin. A modulator includes an agonist, antagonist, inhibitor or inverse agonist, as defined herein. A modulator can be for example, a polypeptide, a peptide, an antibody or antigen-binding fragment thereof, a lipid, a carbohydrate, a nucleic acid, and a small organic molecule. Candidate modulators can be natural or synthetic compounds, including, for example, synthetic small molecules, compounds contained in extracts of animal, plant, bacterial or fungal cells, as well as conditioned medium from such cells.

As used herein, the term “small molecule” refers to a compound having molecular mass of less than 3000 Daltons, preferably less than 2000 or 1500, still more preferably less than 1000, and most preferably less than 600 Daltons. A “small organic molecule” is a small molecule that comprises carbon.

As used herein, the terms “change”, “difference”, “decrease”, or “increase” as applied to e.g., binding or signaling activity or amount of a substance refer to an at least 10% increase or decrease in binding, signaling activity, or for example, level of mRNA, polypeptide or ligand relative to a standard in a given assay.

As used herein, the term “dysregulation” refers to the signaling activity of GPCRx11 in a sample wherein:

a) a 10% or greater increase or decrease in the amount of one or more of GPCRx11 polypeptide, ligand or mRNA level is measured relative to a standard, as defined herein, in a given assay or;

b) at least a single base pair change in the GPCRx11 coding sequence is detected relative to SEQ ID NO: 11, and results in an alteration of GPCRx11 ligand binding or signaling activity as defined in paragraphs a), c) or d) or;

c) a 10% or greater increase or decrease in the amount of GPCRx11 ligand binding activity is measured relative to a standard, as defined herein, in a given assay or;

d) a 10% or greater increase or decrease in a second messenger, as defined herein, is measured relative to the standard, as defined herein, in a given assay.

As used herein, the term “conditions permitting the binding of angiopeptin to a GPCRx11 polypeptide” refers to conditions of, for example, temperature, salt concentration, pH and protein concentration under which angiopeptin binds GPCRx11. Exact binding conditions will vary depending upon the nature of the assay, for example, whether the assay uses viable cells or only a membrane fraction of cells. However, because GPCRx11 is a cell surface protein favored conditions will generally include physiological salt (90 mM) and pH (about 7.0 to 8.0). Temperatures for binding can vary from 15° C. to 37° C., but will preferably be between room temperature and about 30° C. The concentration of angiopeptin in a binding reaction will also vary, but will preferably be about 1 μM (e.g., in a reaction with radiolabelled tracer angiopeptin, where the concentration is generally below the K_{d}) to 10 mM.

As used herein, the term “sample” refers to the source of molecules being tested for the presence of an agent or modulator compound that modulates binding to or signaling activity of a GPCRx11 polypeptide. A sample can be an environmental sample, a natural extract of animal, plant yeast or bacterial cells or tissues, a clinical sample, a synthetic sample, or a conditioned medium from recombinant cells or a fermentation process. The term “tissue sample” refers to a tissue that is tested for the presence, abundance, quality or an activity of a GPCRx11 polypeptide, a nucleic acid encoding a GPCRx11 polypeptide, a GPCRx11 ligand or an agent or compound that modifies the ligand binding or activity of a GPCRx11 polypeptide.

As used herein, a “tissue” is an aggregate of cells that perform a particular function in an organism. The term “tissue” as used herein refers to cellular material from a particular physiological region. The cells in a particular tissue can comprise several different cell types. A non-limiting example of this would be brain tissue that further comprises neurons and glial cells, as well as capillary endothelial cells and blood cells, all contained in a given tissue section or sample. In addition to solid tissues, the term “tissue” is also intended to encompass non-solid tissues, such as blood.

As used herein, the term “membrane fraction” refers to a preparation of cellular lipid membranes comprising a GPCRx11 polypeptide. As the term is used herein, a “membrane fraction” is distinct from a cellular homogenate, in that at least a portion (i.e., at least 10%, and preferably more) of non-membrane-associated cellular constituents has been removed. The term “membrane associated” refers to those cellular constituents that are either integrated into a lipid membrane or are physically associated with a component that is integrated into a lipid membrane.

As used herein, the “second messenger assay” preferably comprises the measurement of guanine nucleotide binding or exchange, adenylate cyclase, intra-cellular cAMP, intracellular inositol phosphate, intra-cellular diacylglycerol concentration, intracellular calcium concentration (e.g., via an aequorin based assay), arachinoid acid concentration, MAP kinase(s) or tyrosine kinase(s), protein kinase C activity, or reporter gene expression according to methods known in the art and defined herein.

As used herein, the term “second messenger” refers to a molecule, generated or caused to vary in concentration by the activation of a G-Protein Coupled Receptor, that participates in the transduction of a signal from that GPCR. Non-limiting examples of second messengers include cAMP, diacylglycerol, inositol triphosphate, arachidonic acid release, inositol triphosphates and intracellular calcium. The term “change in the level of a second messenger” refers to an increase or decrease of at least 10% in the detected level of a given second messenger relative to the amount detected in an assay performed in the absence of a candidate modulator.

As used herein, the term “aequorin-based assay” refers to an assay for GPCR activity that measures elevations in intracellular calcium induced by activated GPCRs, wherein intracellular calcium flux is measured by the luminescence of mitochondiral aequorin expressed in the cell.

As used herein, the term “binding” refers to the physical association of a ligand (e.g., angiopeptin, or an antibody) with a receptor (e.g., GPCRx11). As the term is used herein, binding is “specific” if it occurs with an EC₅₀ or a K_{d} of 1 mM less, generally in the range of 1 mM to 10 nM For example, binding is specific if the EC₅₀ or K_{d} is 1 mM, 500 μM, 100 μM, 10 μM, 9.5 μM, 9 μM, 8.5 μM, 8 μM, 7.5 μM, 7 μM, 6.5 μM, 6 μM, 5.5 μM, 5 μM, 4.5 μM, 4 μM, 3.5 μM, 3 μM, 2.5 μM, 2 μM, 1.5 μM, 1 μM, 750 nM, 500 nM, 250 nM, 100 nNM, or 50 nM or less.

As used herein, the term “EC₅₀,” refers to that concentration of a compound at which a given activity, including binding of angiopeptin or other ligand and a functional activity of a G-protein coupled receptor polypeptide, is 50% of the maximum for that receptor activity measurable using the same assay in the absence of compound. Stated differently, the “EC₅₀” is the concentration of compound that gives 50% activation, when 100% activation is set at the amount of activity that does not increase with the addition of more agonist. It should be noted that the “EC₅₀” of an analog of, for example, angiopeptin, will vary according to the identity of the analogue used in the assay; for example, angiopeptin analogues can have EC₅₀ values higher than, lower than or the same as angiopeptin. Therefore, where an angiopeptin analogue differs from angiopeptin, one of skill in the art can determine the EC₅₀ for that analogue according to conventional methods. The EC₅₀ of a given ligand is measured by performing an assay for the activity of a fixed amount of G-protein coupled receptor polypeptide in the presence of doses of ligand that increase at least until the receptor response is saturated or maximal, and then plotting the measured receptor activity versus the concentration of ligand.

As used herein, the term “saturation” refers to the concentration of angiopeptin or other ligand at which further increases in ligand concentration fail to increase the binding of ligand or G-protein coupled receptor-specific signaling activity.

As used herein, the term “IC₅₀” is the concentration of an antagonist or inverse agonist that reduces the maximal activation of a G-protein coupled receptor by 50%.

As used herein, the term “decrease in binding” refers to a decrease of at least 10% in the amount of ligand binding detected in a given assay with a known or suspected modulator of a G-protein coupled receptor, such as GPCRx11, relative to binding detected in an assay lacking that known or suspected modulator.

As used herein, the term “delivering,” when used in reference to a drug or agent, means the addition of the drug or agent to an assay mixture, or to a cell in culture. The term also refers to the administration of the drug or agent to an animal. Such administration can be, for example, by injection (in a suitable carrier, e.g., sterile saline or water) or by inhalation, or by an oral, transdermal, rectal, vaginal, or other common route of drug administration.

As used herein, the term “standard” refers to a sample taken from an individual who is not affected by a disease or disorder characterized by dysregulation of G-protein coupled receptor (i.e., GPCRx11) activity. The “standard” is used as a reference for the comparison of receptor mRNA or polypeptide levels and quality (i.e., mutant vs. wild type), as well as for the comparison of G-protein coupled receptor activities. A “standard” also encompasses a reference sequence, e.g., SEQ ID NO: 11 or SEQ ID NO: 12, with which sequences of nucleic acids or their encoded polypeptides are compared.

As used herein, the term “amplifying,” when applied to a nucleic acid sequence, refers to a process whereby one or more copies of a nucleic acid sequence is generated from a template nucleic acid. A preferred method of “amplifying” is PCR or RT/PCR.

As used herein, the term “G-Protein coupled receptor,” or “GPCR” refers to a membrane-associated polypeptide with 7 alpha helical transmembrane domains. Functional GPCR's associate with a ligand or agonist and also associate with and activate G-proteins. GPCRx11 is a GPCR.

As used herein, the term “antibody” is the conventional immunoglobulin molecule, as well as fragments thereof which are also specifically reactive with one of the subject polypeptides. Antibodies can be fragmented using conventional techniques and the fragments screened for utility in the same manner as described herein below for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific, single-chain, and chimeric and humanized molecules having affinity for a polypeptide conferred by at least one CDR region of the antibody. In preferred embodiments, the antibody further comprises a label attached thereto and able to be detected, (e.g., the label can be a radioisotope, fluorescent compound, chemiluminescent compound, enzyme, or enzyme co-factor). The antibodies, monoclonal or polyclonal and its hypervariable portion thereof (FAB, FAB″, etc.) as well as the hybridoma cell producing the antibodies are a further aspect of the present invention which find a specific industrial application in the field of diagnostics and monitoring of specific diseases, preferably the ones hereafter described.

Inhibitors according to the invention include but are not limited to labeled monoclonal or polyclonal antibodies or hypervariable portions of the antibodies.

As used herein, the term “transgenic animal” refers to any animal, preferably a non-human mammal, bird, fish or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extra-chromosomally replicating DNA. In the typical transgenic animals described herein, the transgene causes cells to express a recombinant form of one of the subject polypeptide, e.g. either agonistic or antagonistic forms. However, transgenic animals in which the recombinant gene is silent are also contemplated, as for example, the FLP or CRE recombinase dependent constructs described below. Moreover, “transgenic animal” also includes those recombinant animals in which gene disruption of one or more genes is caused by human intervention, including both recombination and antisense techniques.

### BRIEF DESCRIPTION OF THE DRAWINGS

FIG. 1 shows the nucleic aicd and amino acid sequence of GPCRx11 (SEQ ID NO: 11, 12).

FIGS. 2-14 show the nucleic acid and amino acid sequences of GPCRx2 (SEQ ID NO: 1, 2), 5 (SEQ ID NO: 3, 4), 6 (SEQ ID NO: 5, 6), 7 (SEQ ID NO: 7, 8), 9 (SEQ ID NO: 9, 10), 10 (SEQ ID NO: 13, 14) 13 (SEQ ID NO: 15, 16), 14 (SEQ ID NO: 17, 18), 16 (SEQ ID NO: 19, 20), 17 (SEQ ID NO: 21, 22), 18 (SEQ ID NO: 23, 24), 19 (SEQ ID NO: 25, 26), and 20 (SEQ ID NO: 27, 28) respectively.

FIG. 15 shows the dendrogram constructed based on the alignment of the amino acid sequence of GPCRx11 with RTA and other RTA related sequences.

FIG. 16 shows the dose response curve of angiopeptin stimulation of aequorin fluorescence measured from transfected CHO-K1 cells.

### DETAILED DESCRIPTION

The invention is further based on the identification of 13 new G-protein coupled receptors, methods of identifying molecules which bind the receptors, and methods of identifying ligands of the receptors. The invention is further based on the discovery that angiopeptin are natural ligands for the one of these G protein coupled receptors, GPCRx11, and on methods of using the binding of this ligand to the receptor in drug screening methods. The known ligand and its interaction with the receptor GPCRx11 also provides for the diagnosis of conditions involving dysregulated receptor activity. The invention also relates to a kit comprising GPCRx11 and homologous sequences, its corresponding polynucleotide and/or recombinant cells expressing the polynucleotide, to identify agonist, antagonist and inverse agonist compounds of the receptor polypeptide and/or its corresponding polynucleotide. Such kits are useful for the diagnosis, prevention and/or a treatment of diseases and disorders related to GPCRx11 activity.

The invention also relates to novel agonist, antagonist and inverse agonist compounds of the receptor polypeptide and its corresponding polynucleotide, identified according to the method of the invention.

All references referred to below and above are incorporated herein by reference in their entirety. Sequences The invention relates to the nucleotide (SEQ ID NO: 1, 3, 5, 7, 9, 11, 13, 15, 17, 19, 21, 23, 25, and 27) and amino acid (SEQ ID NO: 2, 4, 6, 8, 10, 12, 14, 16, 18, 20, 22, 24, 26, and 28) sequences encoding the G-protein coupled recpetors according to the invention, and in particular, the nucleotide sequence of SEQ ID NO: 11 which encodes the polypeptide sequence of GPCRx11 (SEQ ID NO: 12). Table 1 below shows the designation of each of the G-protein coupled receptors of the invention and their corresponding SEQ ID NO.

TABLE 1

| | Nucleic Acid | Amino Acid SEQ |
|---|---|---|
| Receptor Name | SEQ ID NO. | ID NO. |
| GPCRx2 | 1 | 2 |
| GPCRx5 | 3 | 4 |
| GPCRx6 | 5 | 6 |
| GPCRx7 | 7 | 8 |
| GPCRx9 | 9 | 10 |
| GPCRx10 | 13 | 14 |
| GPCRx11 | 11 | 12 |
| GPCRx13 | 15 | 16 |
| GPCRx14 | 17 | 18 |
| GPCRx16 | 19 | 20 |
| GPCRx17 | 21 | 22 |
| GPCRx18 | 23 | 24 |
| GPCRx19 | 25 | 26 |
| GPCRx20 | 27 | 28 |

The invention also relates to sequences that are homologous to the nucleotide and amino acid sequences encoding the GPCRs of Table 1.

Calculation of Sequence Homology

Sequence identity with respect to any of the sequences presented herein can be determined by a simple “eyeball” comparison (i.e. a strict comparison) of any one or more of the sequences with another sequence to see if that other sequence has, for example, at least 80% sequence identity to the sequence(s).

Relative sequence identity can also be determined by commercially available computer programs that can calculate % identity between two or more sequences using any suitable algorithm for determining identity, using for example default parameters. A typical example of such a computer program is CLUSTAL. Other computer program methods to determine identity and similarity between two sequences include but are not limited to the GCG program package (Devereux et al 1984 Nucleic Acids Research 12: 387) and FASTA (Atschul et al 1990 J Molec Biol 403-410).

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalizing unduly the overall homology score. This is achieved by inserting “gaps” in the sequence alignment to try to maximize local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimized alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software that can perform sequence comparisons include, but are not limited to, the BLAST package (Ausubel et al., 1995, Short Protocols in Molecular Biology, 3rd Edition, John Wiley & Sons), FASTA (Atschul et al., 1990, J. Mol. Biol., 403-410) and the GENEWORKS suite of comparison tools. Both BLAST and FASTA are available for offline and online searching (Ausubel et al., 1999 supra, pages 7-58 to 7-60).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied. It is preferred to use the public default values for the GCG package, or in the case of other software, the default matrix, such as BLOSUM62.

Advantageously, the BLAST algorithm is employed, with parameters set to default values. The BLAST algorithm is described in detail in Altschul et al., (1990) J. Mol. Biol. 215:403-410, which is incorporated herein by reference. The search parameters are defined as follows, and can be advantageously set to the defined default parameters.

Advantageously, “substantial identity” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul (Karlin and Altschul 1990, Proc. Natl. Acad. Sci. USA 87:2264-68; Karlin and Altschul, 1993, Proc. Natl. Acad. Sci. USA 90:5873-7) with a few enhancements. The BLAST programs are tailored for sequence similarity searching, for example to identify homologues to a query sequence. For a discussion of basic issues in similarity searching of sequence databases, see Altschul et al (1994) Nature Genetics 6:119-129.

The five BLAST programs available through the National Institutes of Health (NIH; Bethesda, Md.) perform the following tasks: blastp—compares an amino acid query sequence against a protein sequence database; blastn—compares a nucleotide query sequence against a nucleotide sequence database; blastx—compares the six-frame conceptual translation products of a nucleotide query sequence (both strands) against a protein sequence database; tblastn—compares a protein query sequence against a nucleotide sequence database dynamically translated in all six reading frames (both strands); tblastx—compares the six-frame translations of a nucleotide query sequence against the six-frame translations of a nucleotide sequence database.

BLAST uses the following search parameters:

HISTOGRAM—Display a histogram of scores for each search; default is yes. (See parameter H in the BLAST Manual).

DESCRIPTIONS —Restricts the number of short descriptions of matching sequences reported to the number specified; default limit is 100 descriptions. (See parameter V in the manual page).

EXPECT—The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF—Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

ALIGNMENTS—Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

MATRIX—Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND—Restrict a TBLASTN search to just the top or bottom strand of the database sequences; or restrict a BLASTN, BLASTX or TBLASTX search to just reading frames on the top or bottom strand of the query sequence.

FILTER—Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Claverie & States (1993) Computers and Chemistry 17:191-201, or, for BLASTN, by the DUST program of Tatusov and Lipman (NIH). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi—Causes NCBI gi identifiers to be shown in the output, in addition to the accession and/or locus name.

Most preferably, sequence comparisons are conducted using the simple BLAST search algorithm provided by the NIH. In some embodiments of the present invention, no gap penalties are used when determining sequence identity.

Vectors and Host Cells

In one embodiment, the present invention provides both vector constructs comprising a nucleic acid sequence encoding the G-protein coupled receptors of the present invention, and one or more host cells comprising such a vector.

A “vector” for purposes of the present invention may be any vector known to those of skill in the art such as a plasmid or viral vector, into which a sequence of the invention (e.g., SEQ ID NO: 11) has been inserted, in a forward or reverse orientation. The construct also will include regulatory sequences, including, for example, a promoter, operably linked to the sequence. Large numbers of suitable vectors and promoters are known to those of skill in the art, and are commercially available. The following vectors are provided by way of example. Bacterial: pQE70, pQE60, pQE-9 (Qiagen) pBs, phagescript, psiX174, pBluescript SK, pBsKS, pNH8a, pNH16a, pNH18a, pNH46a (Stratagene); pTrc99A, pKK223-3, pKK233-3, pDR540, pRIT5 (Pharmacia). Eukaryotic: pWLneo, pSV2cat, pOG44, pXT1, pSG (Stratagene) pSVK3, pBPV, pMSG, pSVL (Pharmacia). However, any other plasmid or vector may be used as long as it is replicable and viable in the host.

Promoter regions can be selected from any characterized gene and incorporated into appropriate vectors using techniques well known in the art. Two appropriate vectors are pKK232-8 and pCM7. Particular named bacterial promoters include lac, lacZ, T3, T7, gpt, P_{L} and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

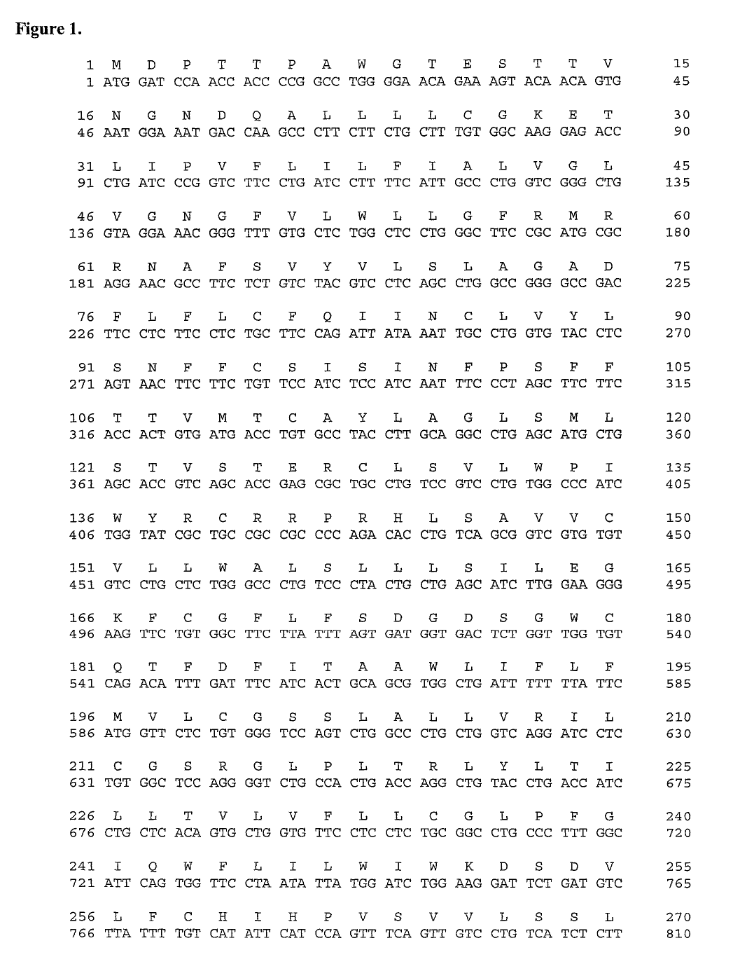

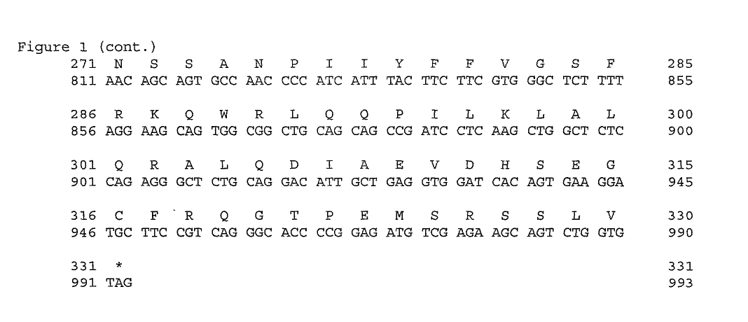

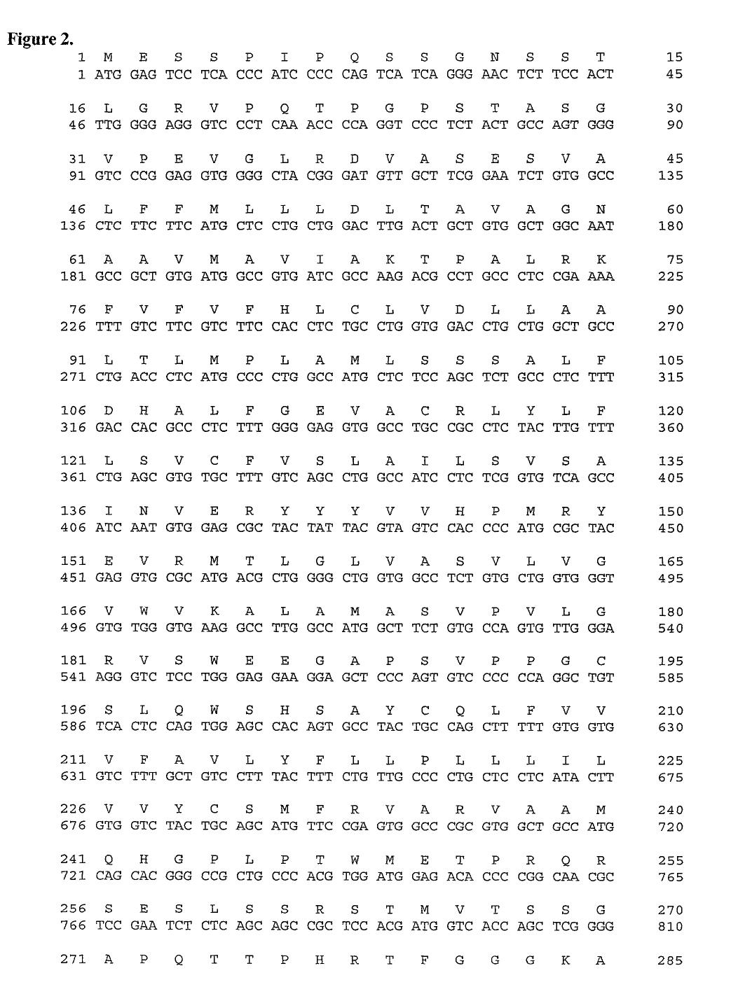

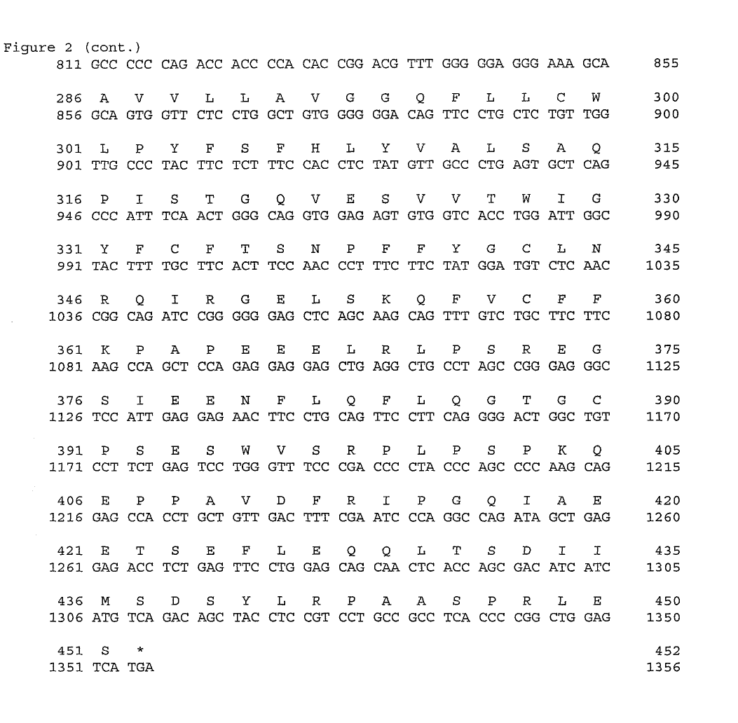

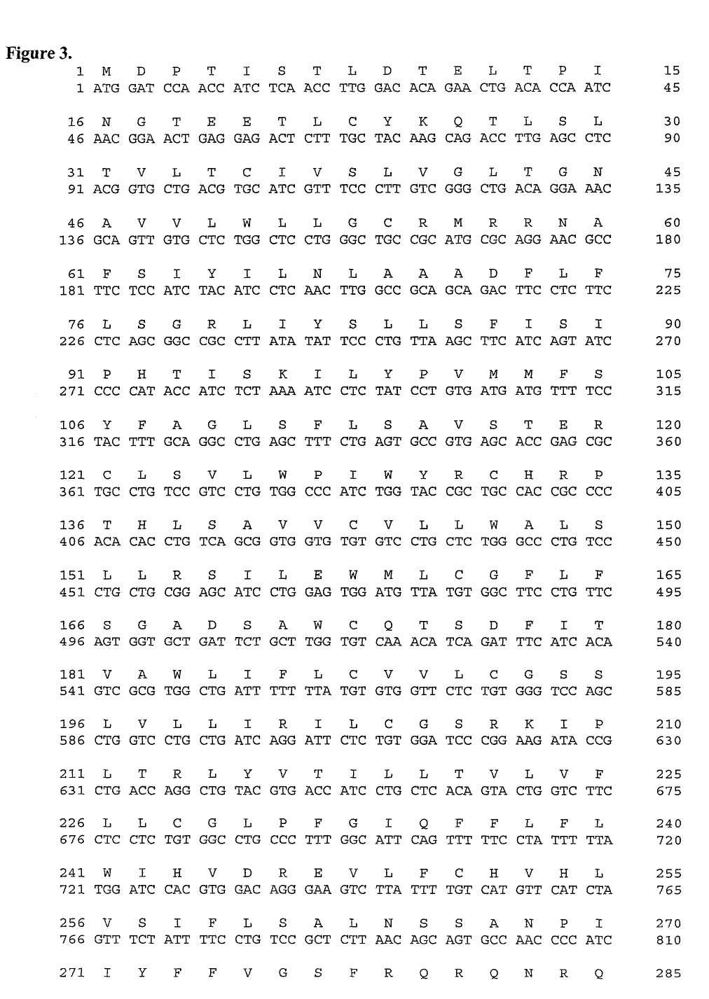

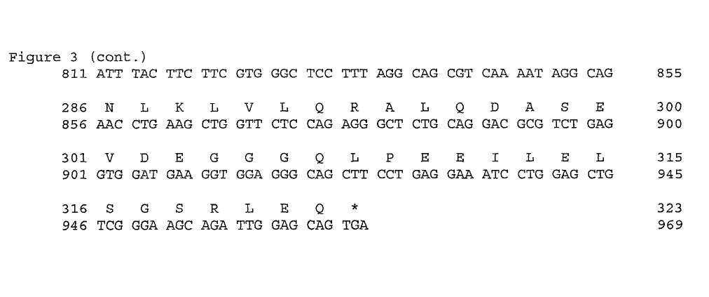

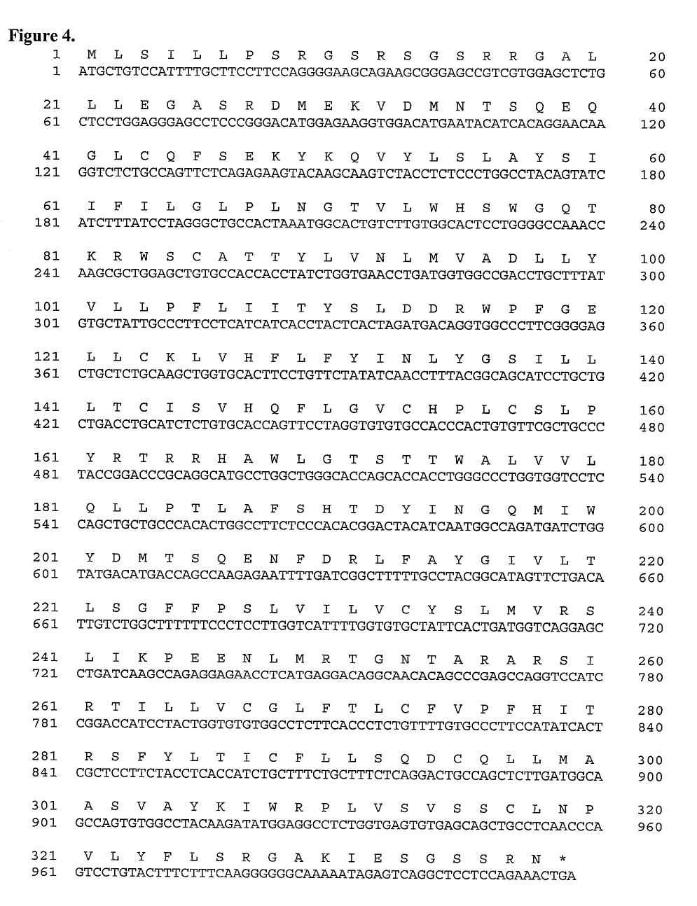

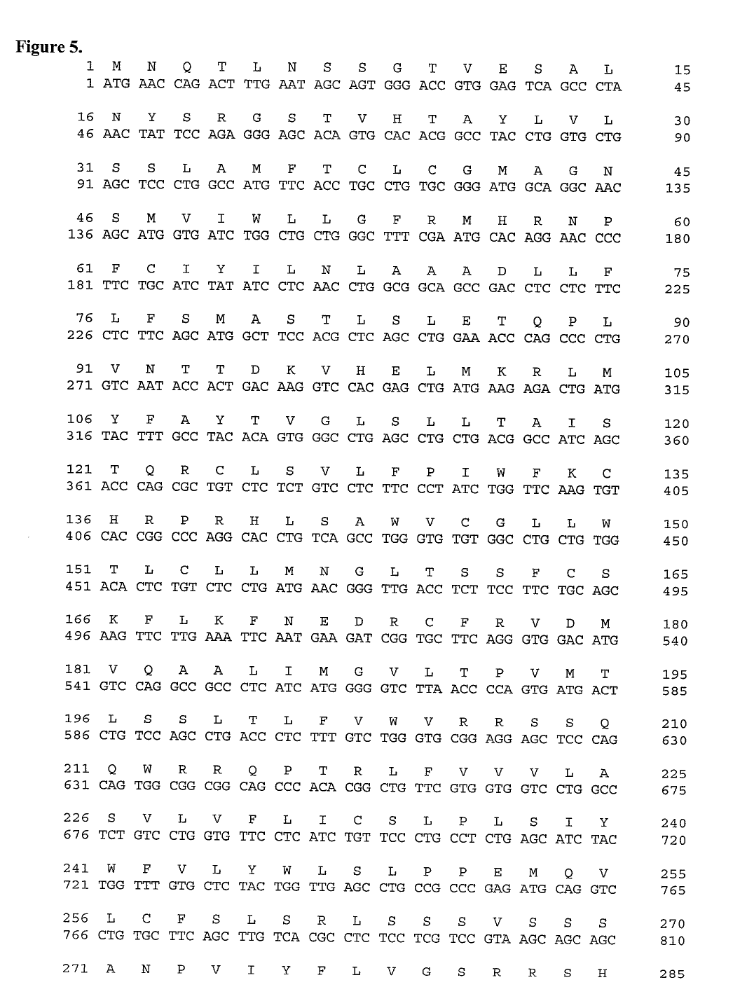

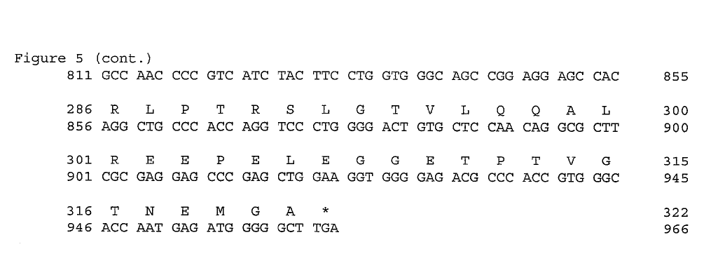

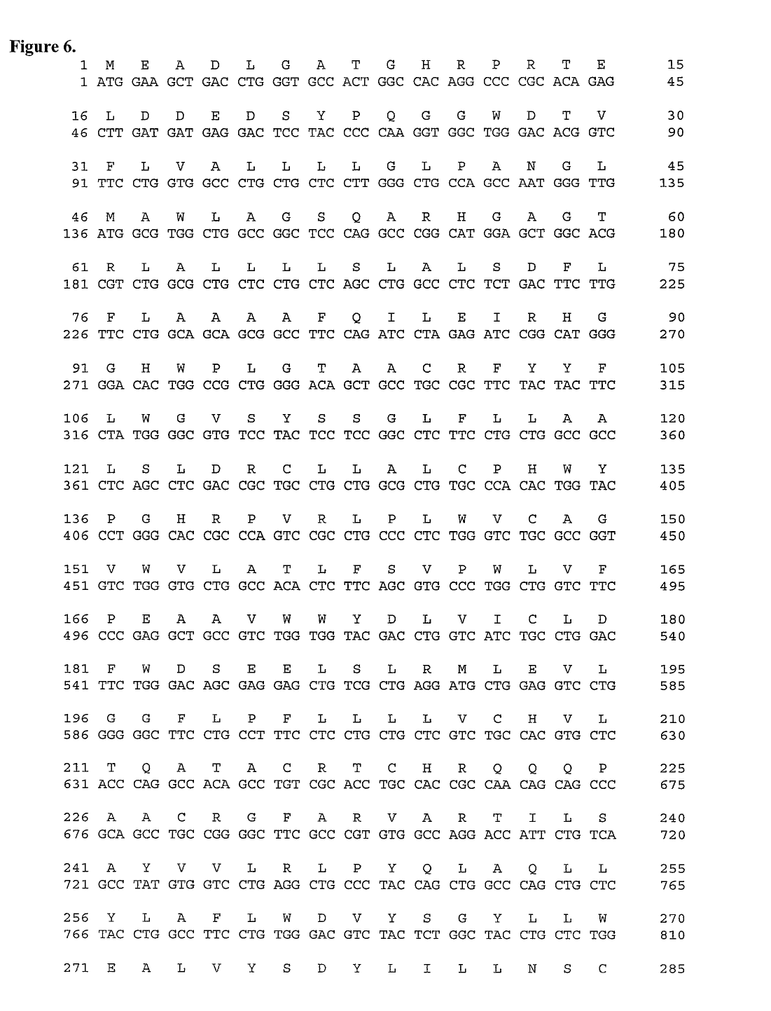

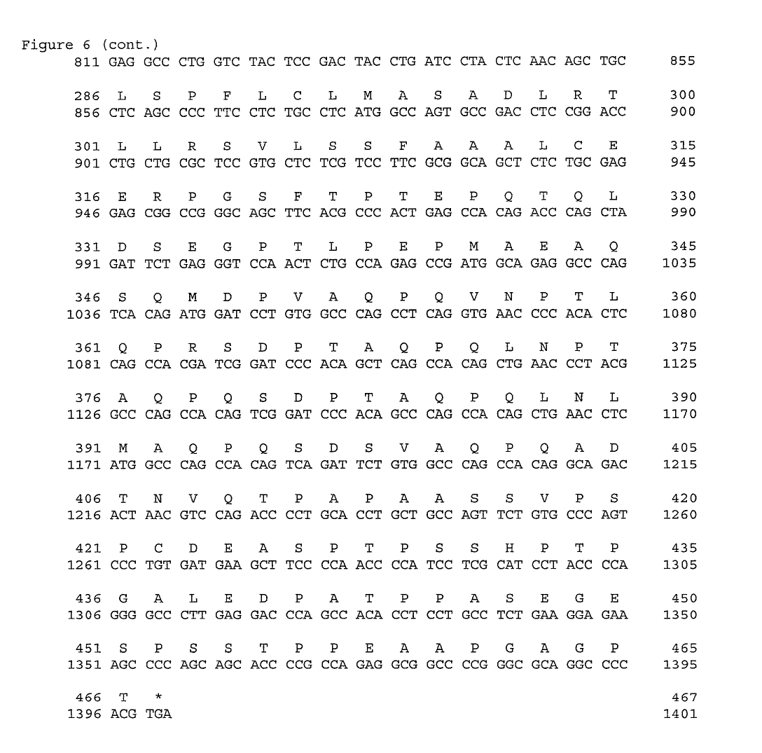

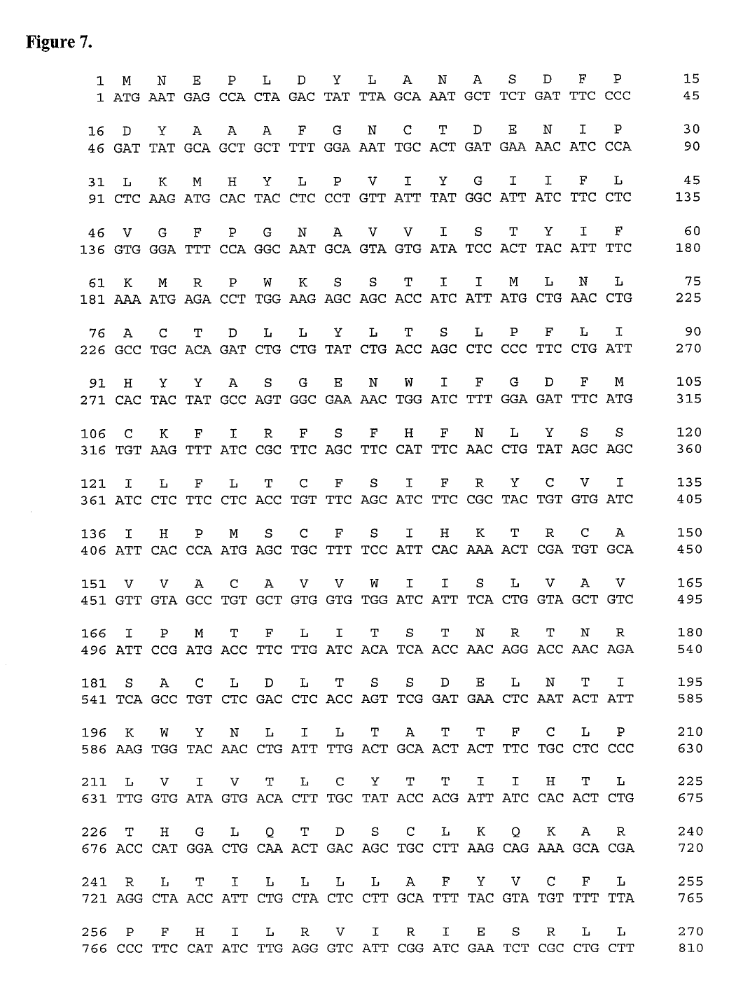

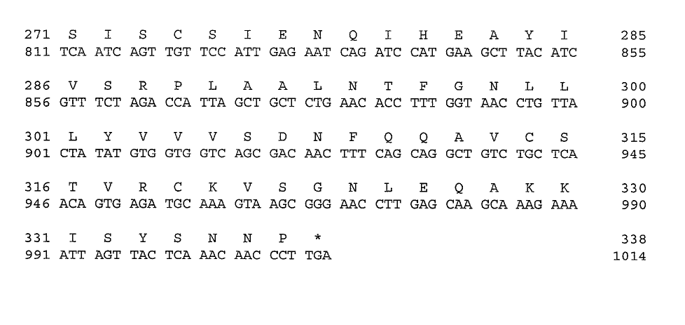

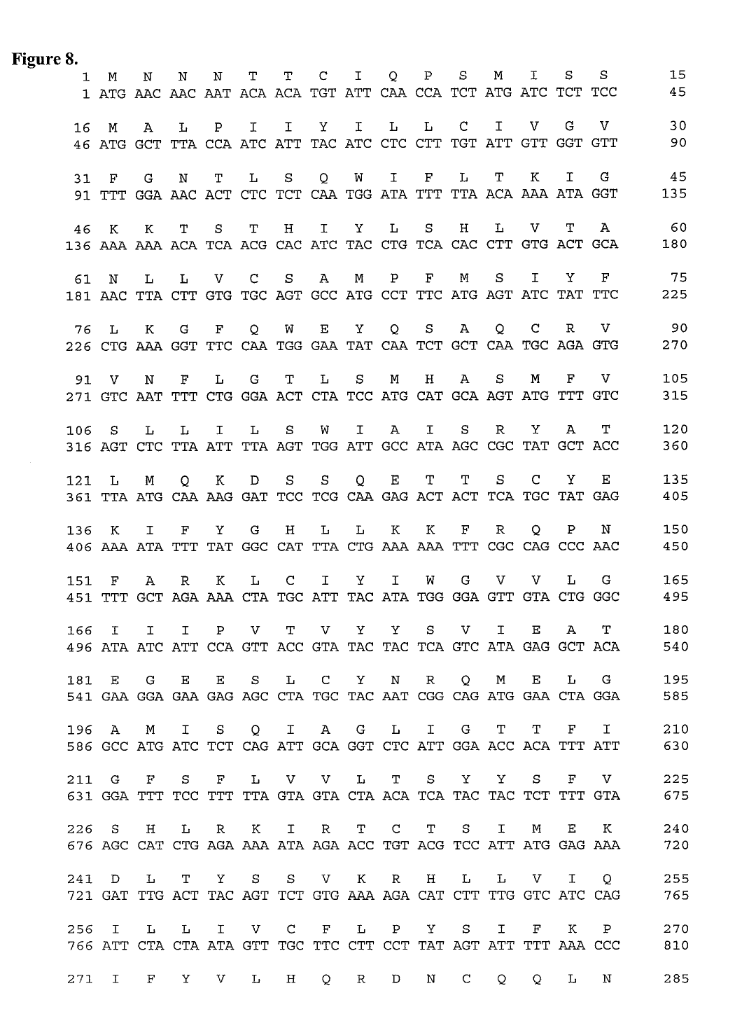

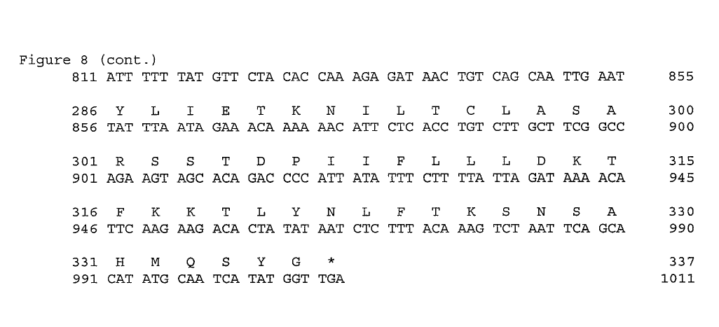

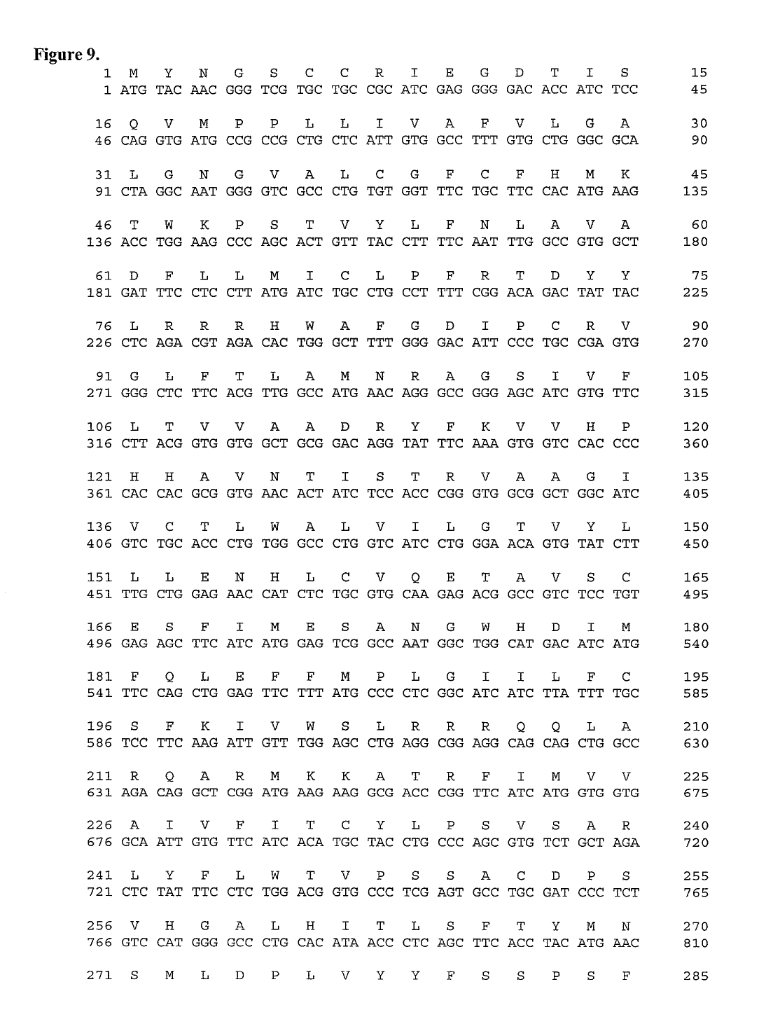

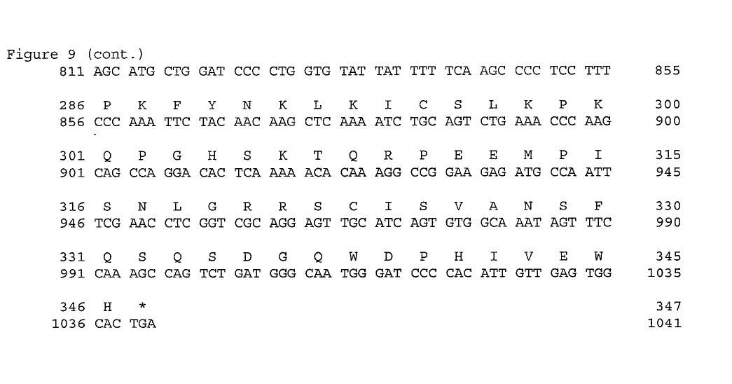

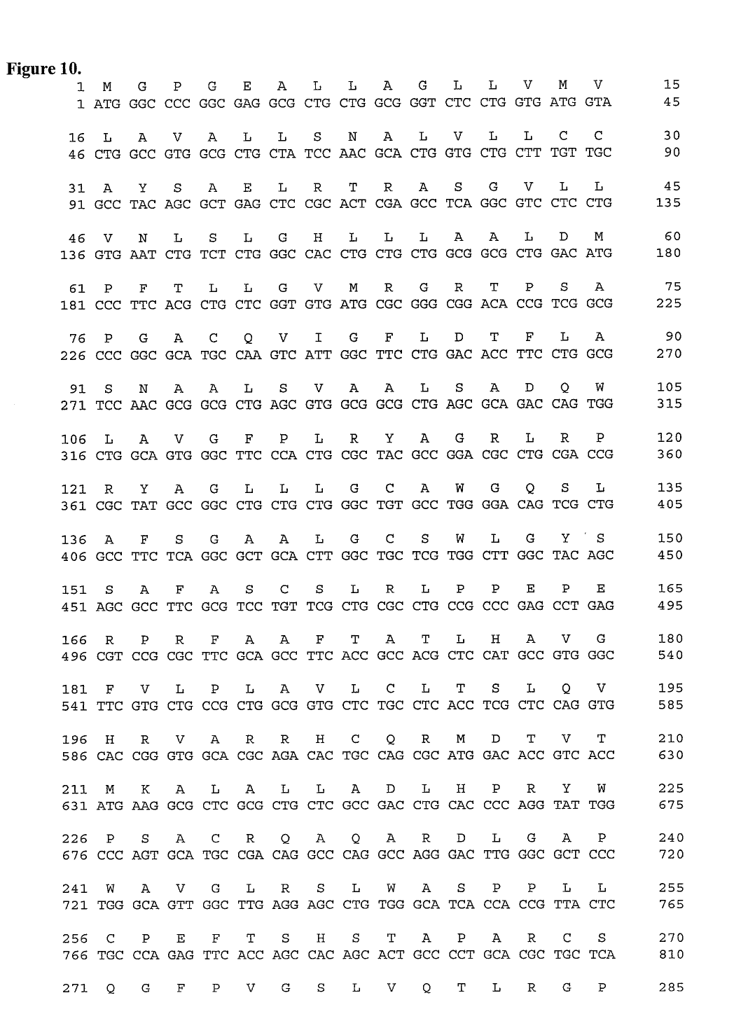

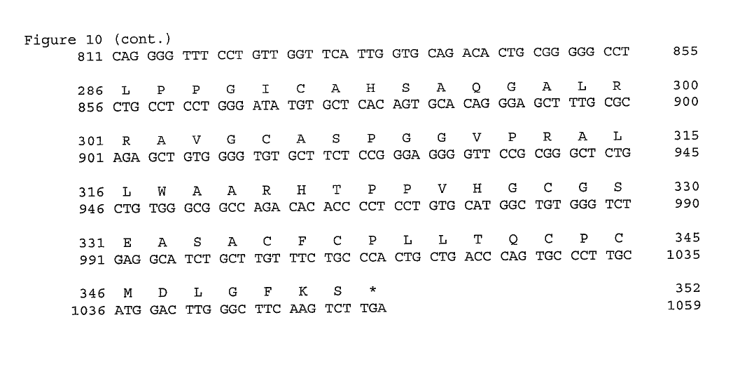

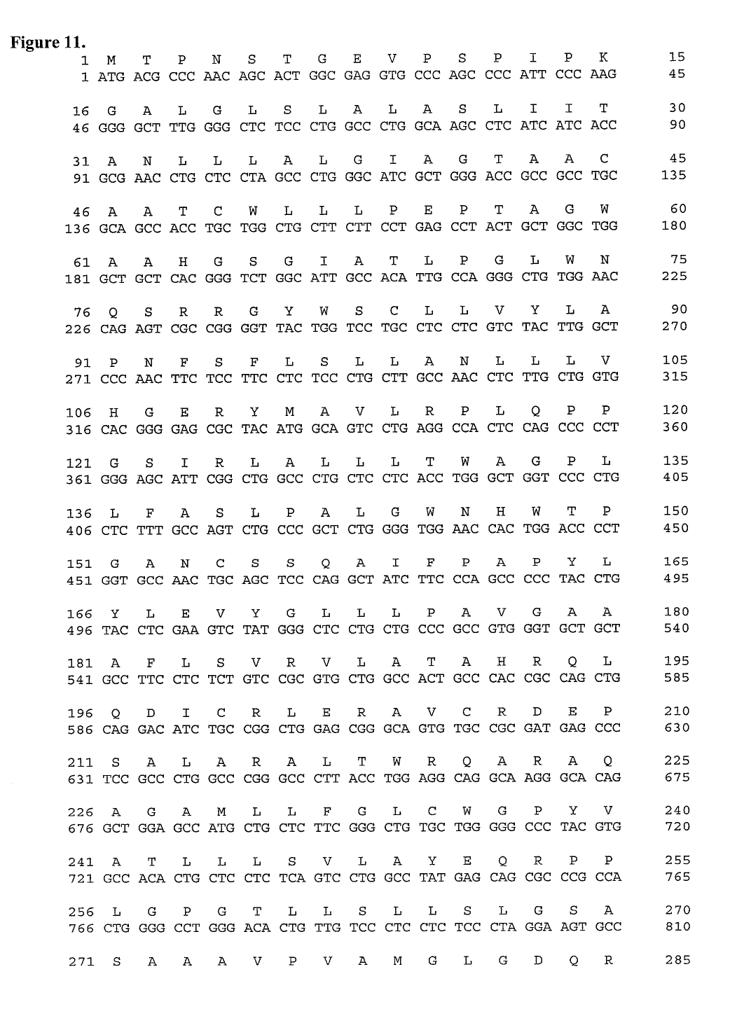

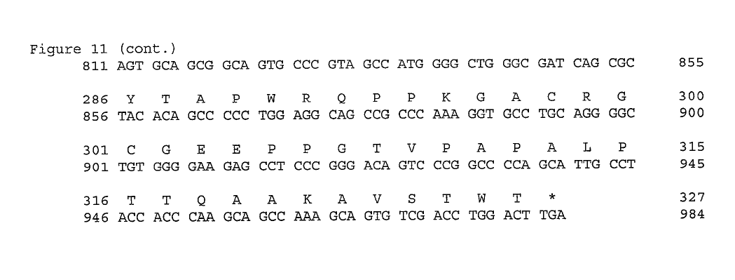

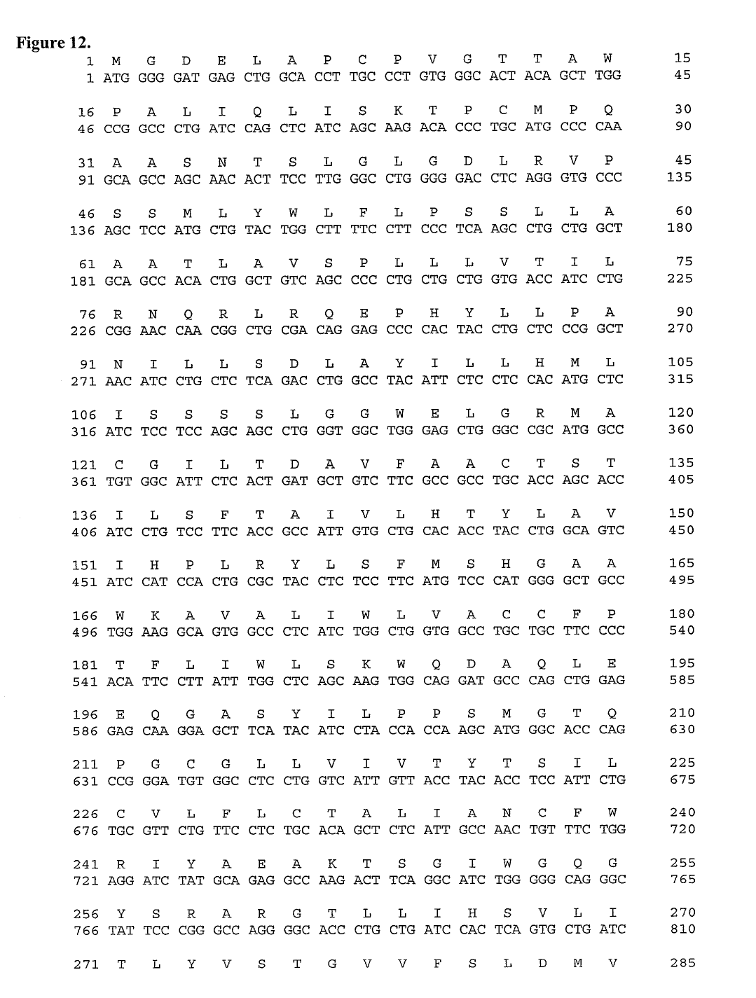

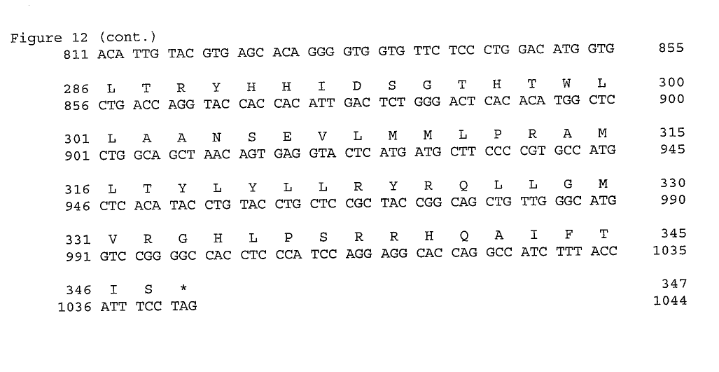

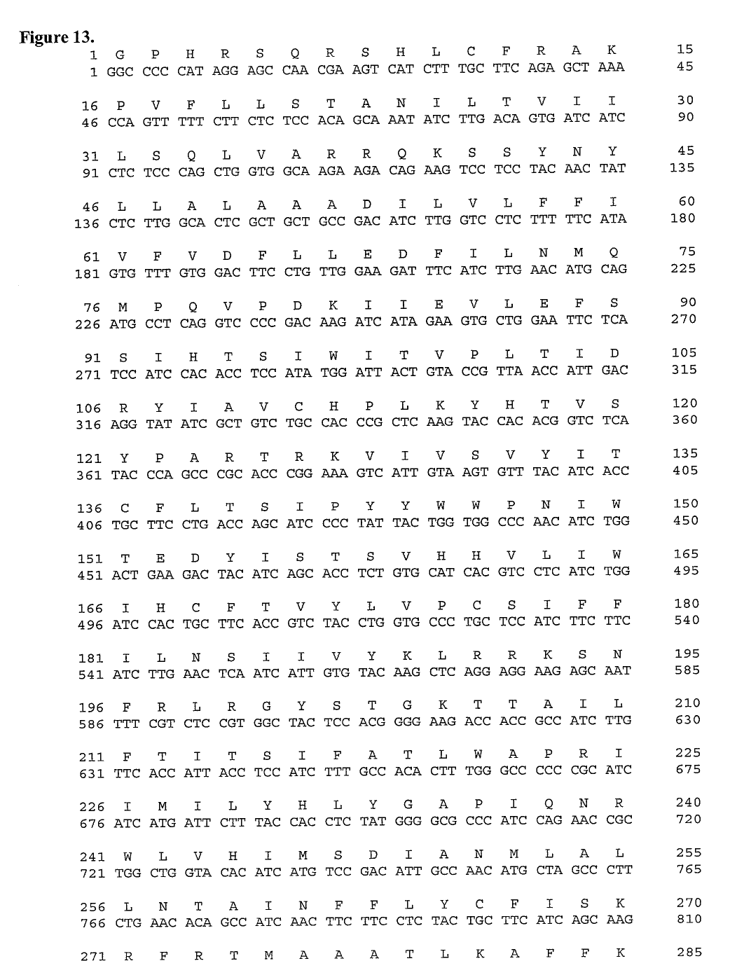

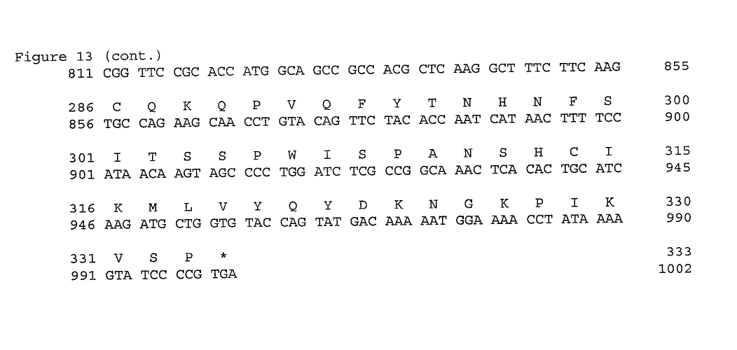

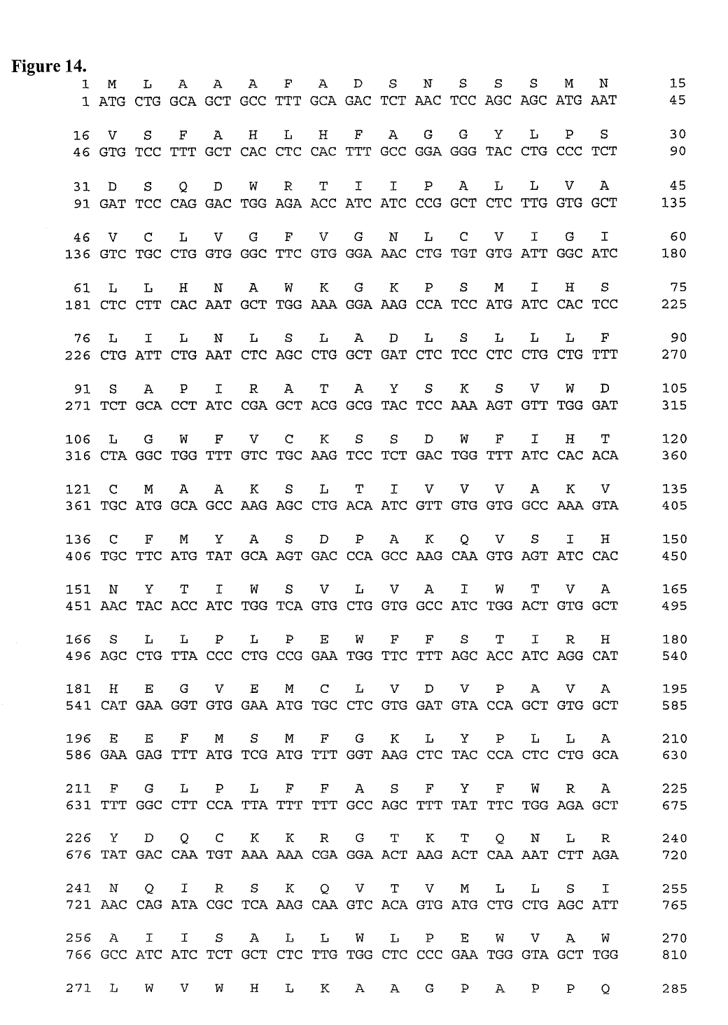

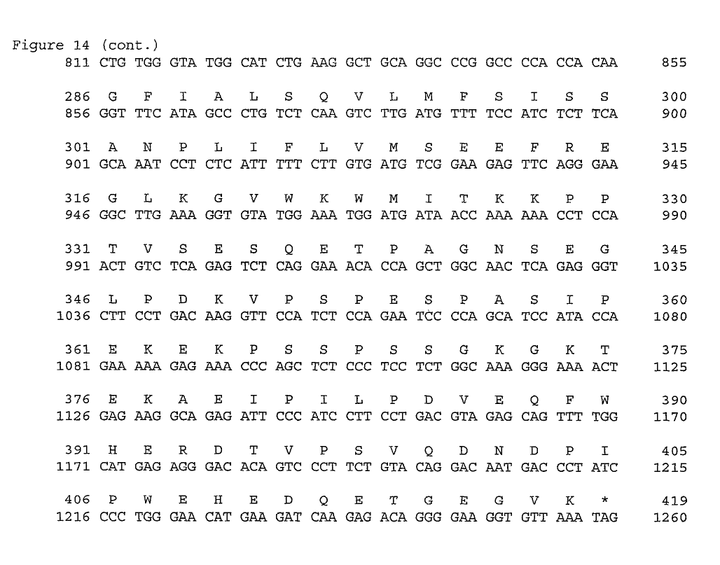

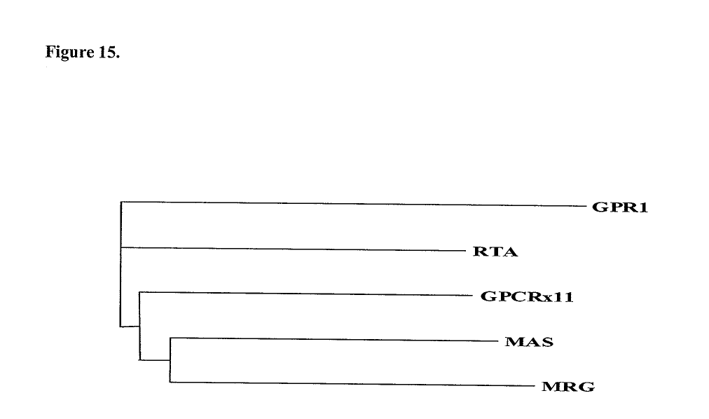

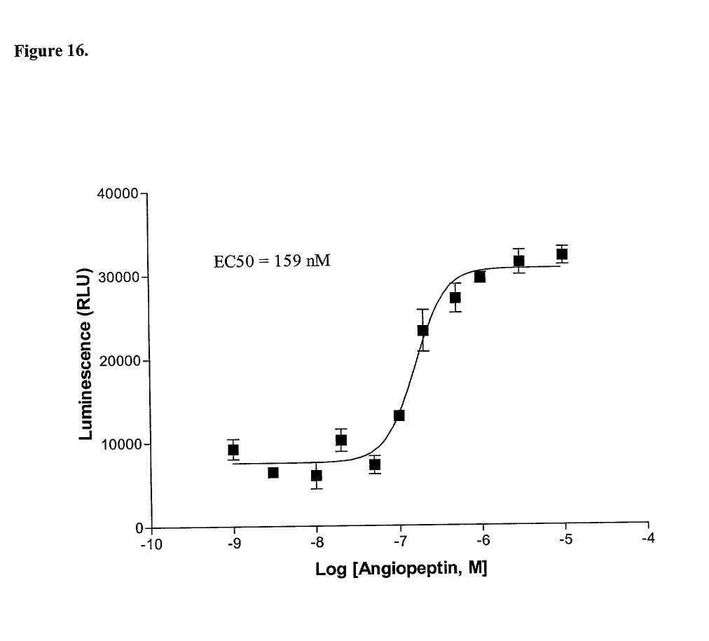

A host cell containing an above-described construct may be a higher eukaryotic cell, such as a mammalian cell, or a lower eukaryotic cell, such as a yeast cell, or the host cell may be a prokaryotic cell, such as a bacterial cell. A host cell that is useful according to the invention can be any cell into which a nucleic acid sequence encoding a receptor according to the invention can be introduced such that the receptor is expressed at natural levels or above natural levels, as defined herein. Preferably a receptor of the invention that is expressed in a cell exhibits normal or near normal pharmacology, as defined herein. Most preferably a receptor of the invention that is expressed in a cell comprises the nucleotide or amino acid sequence presented in FIG. 1 or a nucleotide or amino acid sequence that is at least 70% identical to the amino acid sequence presented in FIG. 1.

According to a preferred embodiment of the present invention, a cell is selected from the group consisting of COS7-cells, a CHO cell, a LM (TK-) cell, a NIH-3T3 cell, HEK-293 cell, K-562 cell or a 1321N1 astrocytoma cell but also other transfectable cell lines.

Introduction of the construct into the host cell can be effected by calcium phosphate transfection, DEAE-Dextran mediated transfection, liposome mediated transfection, or electroporation (Ausubel et al., supra, 1992, pp. 9-5 to 9-14). The constructs in host cells can be used in a conventional manner to produce the gene product encoded by the recombinant sequence (i.e., GPCRx11). Alternatively, the polypeptides of the invention can be synthetically produced by conventional peptide synthesizers.

Polypeptides can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook et al., Molecular Cloning: A Laboratory Manual, Second Edition, 1989, (Cold Spring Harbor Press, Cold Spring Harbor, N.Y.), the disclosure of which is hereby incorporated by reference.

Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), alpha factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

Transcription of a DNA encoding the polypeptides of the present invention by higher eukaryotes is increased by inserting an enhancer sequence into the vector. Enhancers are cis-acting elements of DNA, usually about from 10 to 300 bp, that act on a promoter to increase its transcription. Examples include the SV40 enhancer on the late side of the replication origin (bp 100 to 270), a cytomegalovirus early promoter enhancer, a polyoma enhancer on the late side of the replication origin, and adenovirus enhancers. Useful expression vectors for bacterial use are constructed by inserting a structural DNA sequence encoding a desired protein together with suitable translation initiation and termination signals in operable reading phase with a functional promoter. The vector may include one or more phenotypic selectable markers and an origin of replication to ensure maintenance of the vector and, if desirable, to provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice. As a representative but nonlimiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, PKK223-3 (Pharmacia Fine Chemicals, Uppsala, Sweden) and GEM1 (Promega, Madison, Wis.). These pBR322 “backbone” sections are combined with an appropriate promoter and the structural sequence to be expressed.

Following transformation of a suitable host strain and growth of the host strain to an appropriate cell density, the selected promoter is de-repressed by appropriate means (e.g., temperature shift or chemical induction) and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents. Such methods are well-known to those skilled in the art.

Various mammalian cell culture systems can also be employed to express recombinant protein. Examples of mammalian expression systems include the COS-7 lines of monkey kidney fibroblasts, described by Gluzman, 1981, Cell, 23:175, and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, a polyadenylation site, splice donor and acceptor sites, transcriptional termination sequences, and 5′ flanking nontranscribed sequences. DNA sequences derived from the SV40 viral genome, for example, SV40 origin, early promoter, enhancer, splice, and polyadenylation sites, may be used to provide the required nontranscribed genetic elements. The expressed recombinant protein encoded by the gene sequence comprising the polynucleotide of the invention may be isolated, if necessary, by means known to those skilled in the art.

Assays

I. Assays for the Identification of Agents that Modulate the Activity of a G-protein Coupled Receptor

Agents that act as ligands, or modulate the activity of a G-protein coupled receptor according to the invention can be identified in a number of ways. In one embodiment, this identification takes advantage of the newly discovered interaction of the GPCRx11 with angiopeptin. For example, the ability to reconstitute GPCRx11/angiopeptin binding either in vitro, on cultured cells or in vivo provides a target for the identification of agents that disrupt that binding. Assays based on disruption of binding can identify agents, such as small organic molecules, from libraries or collections of such molecules. Alternatively, such assays can identify agents in samples or extracts from natural sources, e.g., plant, fungal or bacterial extracts or even in human tissue samples (e.g., tumor tissue). In one aspect, the extracts can be made from cells expressing a library of variant nucleic acids, peptides or polypeptides. Modulators of GPCRx11/angiopeptin binding can then be screened using a binding assay or a functional assay that measures downstream signaling through the receptor.

Another approach that uses the GPCRx11/angiopeptin interaction more directly to identify agents that modulate GPCRx11 function measures changes in GPCRx11 downstream signaling induced by candidate agents or candidate modulators. These functional assays can be performed in isolated cell membrane fractions or on cells expressing the receptor on their surfaces.

The discovery that angiopeptin is a ligand of the GPCRx11 receptor permits screening assays to identify agonists, antagonists and inverse agonists of receptor activity. In another embodiment, the invention provides screening assays to identify molecules which bind to the G-protein coupled receptors of the present invention, and to identify potential ligands of the receptors disclosed herein. The screening assays have two general approaches, detailed below.

1) Ligand binding assays, in which cells expressing a G-protein coupled receptor, membrane extracts from such cells, or immobilized lipid membranes comprising a G-protein coupled receptor (preferably GPCRx11) are exposed to a labeled candidate compound. Following incubation, the reaction mixture is measured for specific binding of the labeled candidate compound to the G-protein coupled receptor. In one embodiment, compounds that interfere with binding or displace labeled ligand (i.e., angiopeptin) can be agonists, antagonists or inverse agonists of GPCRx11 activity. Subsequent functional analysis can then be performed on positive compounds to determine in which of these categories they belong.

2) Functional assays, in which a signaling activity of a G-protein coupled receptor, preferably GPCRx11, is measured.

a) For agonist or ligand screening, cells expressing a G-protein coupled receptor or membranes prepared from them are incubated with a candidate compound, and a signaling activity of the receptor is measured. For receptors for which the natural ligand is not known (i.e., GPCRx2, 5, 6, 7, 9, 13, 14, 16, 17, 18, 19, and 20), the detection of a signaling activity upon contacting the receptor with a candidate compound is indicative of the candidate compound being a potential ligand for the particular receptor.

In the case of GPCRx11, the activity induced by compounds that modulate receptor activity is compared to that induced by the natural ligand, angiopeptin. An agonist or partial agonist will have a maximal biological activity corresponding to at least 10% of the maximal activity of angiopeptin when the agonist or partial agonist is present at 1 mM or less, and preferably will have a potency which is at least as potent as angiopeptin.

b) For antagonist or inverse agonist screening, cells expressing GPCRx11 or membranes isolated from them are assayed for signaling activity in the presence of angiopeptin with or without a candidate compound. Antagonists will reduce the level of angiopeptin-stimulated receptor activity by at least 10%, relative to reactions lacking the antagonist in the presence of angiopeptin. Inverse agonists will reduce the constitutive activity of the receptor by at least 10%, relative to reactions lacking the inverse agonist.

c) For inverse agonist screening, cells expressing constitutive GPCRx11 activity or membranes isolated from them are used in a functional assay that measures an activity of the receptor in the presence of a candidate compound. Inverse agonists are those compounds that reduce the constitutive activity of the receptor by at least 10%. Overexpression of GPCRx11 may lead to constitutive activation. GPCRx11 can be overexpressed by placing it under the control of a strong constitutive promoter, e.g., the CMV early promoter. Alternatively, certain mutations of conserved GPCR amino acids or amino acid domains tend to lead to constitutive activity. See for example: Kjelsberg et al., 1992, J. Biol. Chem. 267:1430; McWhinney et al., 2000. J. Biol. Chem. 275:2087; Ren et al., 1993, J. Biol. Chem. 268:16483; Samama et al., 1993, J.Biol.Chem 268:4625; Parma et al., 1993, Nature 365:649; Parma et al., 1998, J. Pharmacol. Exp.Ther. 286:85; and Parent et al., 1996, J. Biol. Chem. 271:7949.

Ligand Binding and Displacement Assays

As noted in (1) above, one can use G-protein coupled receptor polypeptides expressed on a cell, or isolated membranes containing receptor polypeptides, along with angiopeptin (in the case of GPCRx11) in order to screen for compounds that bind to the G-protein coupled receptor, or in an alternate embodiment, compounds which inhibit the binding of angiopeptin to GPCRx11.

For displacement experiments, cells expressing a GPCRx11 polypeptide (generally 25,000 cells per assay or 1 to 100 μg of membrane extracts) are incubated in binding buffer with labeled angiopeptin in the presence or absence of increasing concentrations of a candidate modulator. To validate and calibrate the assay, control competition reactions using increasing concentrations of unlabeled angiopeptin can be performed. After incubation, cells are washed extensively, and bound, labeled angiopeptin is measured as appropriate for the given label (e.g., scintillation counting, fluorescence, etc.). A decrease of at least 10% in the amount of labeled angiopeptin bound in the presence of candidate modulator indicates displacement of binding by the candidate modulator. Candidate modulators are considered to bind specifically in this or other assays described herein if they displace 50% of labeled angiopeptin (sub-saturating angiopeptin dose) at a concentration of 1 mM or less.

Alternatively, binding or displacement of binding can be monitored by surface plasmon resonance (SPR). Surface plasmon resonance assays can be used as a quantitative method to measure binding between two molecules by the change in mass near an immobilized sensor caused by the binding or loss of binding of angiopeptin from the aqueous phase to a GPCRx11 polypeptide immobilized in a membrane on the sensor, or in the case of the other GPCRs disclosed herein, the binding of a candidate molecule from the aqueous phase to the GPCR. This change in mass is measured as resonance units versus time after injection or removal of the angiopeptin or candidate molecule and is measured using a Biacore Biosensor (Biacore AB). A G-protein coupled receptor can be immobilized on a sensor chip (for example, research grade CM5 chip; Biacore AB) in a thin film lipid membrane according to methods described by Salamon et al. (Salamon et al., 1996, Biophys J. 71: 283-294; Salamon et al., 2001, Biophys. J. 80: 1557-1567; Salamon et al., 1999, Trends Biochem. Sci. 24: 213-219, each of which is incorporated herein by reference.). Sarrio et al. demonstrated that SPR can be used to detect ligand binding to the GPCR A(1) adenosine receptor immobilized in a lipid layer on the chip (Sarrio et al., 2000, Mol. Cell. Biol. 20: 5164-5174, incorporated herein by reference). Conditions for candidate molecule binding to a G-protein coupled receptor in an SPR assay can be fine-tuned by one of skill in the art using the conditions reported by Sarrio et al. as a starting point.

In one aspect, SPR can also be used to assay for modulators of angiopeptin binding to GPCRx11 in at least two ways. First, angiopeptin can be pre-bound to immobilized GPCRx11 polypeptide, followed by injection of candidate modulator at a concentration ranging from 0.1 nM to 1 μM. Displacement of the bound angiopeptin can be quantitated, permitting detection of modulator binding. Alternatively, the membrane-bound GPCRx11 polypeptide can be pre-incubated with candidate modulator and challenged with angiopeptin. A difference in angiopeptin binding to the GPCRx11 exposed to modulator relative to that on a chip not pre-exposed to modulator will demonstrate binding or displacement of angiopeptin in the presence of modulator. In either assay, a decrease of 10% or more in the amount of angiopeptin bound is in the presence of candidate modulator, relative to the amount of a angiopeptin bound in the absence of candidate modulator indicates that the candidate modulator inhibits the interaction of GPCRx11 and angiopeptin.

Another method of detecting inhibition of binding of angiopeptin to GPCRx11 uses fluorescence resonance energy transfer (FRET). FRET is a quantum mechanical phenomenon that occurs between a fluorescence donor (D) and a fluorescence acceptor (A) in close proximity to each other (usually <100 A of separation) if the emission spectrum of D overlaps with the excitation spectrum of A. The molecules to be tested, e.g. angiopeptin and a GPCRx11 polypeptide, are labeled with a complementary pair of donor and acceptor fluorophores. While bound closely together by the GPCRx11 :angiopeptin interaction, the fluorescence emitted upon excitation of the donor fluorophore will have a different wavelength than that emitted in response to that excitation wavelength when the angiopeptin and GPCRx11 polypeptide are not bound, providing for quantitation of bound versus unbound molecules by measurement of emission intensity at each wavelength. Donor fluorophores with which to label the GPCRx11 polypeptide are well known in the art. Of particular interest are variants of the A. victoria GFP known as Cyan FP (CFP, Donor (D)) and Yellow FP (YFP, Acceptor(A)). As an example, the YFP variant can be made as a fuision protein with GPCRx11. Vectors for the expression of GFP variants as fusions (Clontech) as well as flurophore-labeled angiopeptin compounds (Molecular Probes) are known in the art. The addition of a candidate modulator to the mixture of labeled angiopeptin and YFP-GPCRx11 protein will result in an inhibition of energy transfer evidenced by, for example, a decrease in YFP fluorescence relative to a sample without the candidate modulator. In an assay using FRET for the detection of GPCRx11 angiopeptin interaction, a 10% or greater decrease in the intensity of fluorescent emission at the acceptor wavelength in samples containing a candidate modulator, relative to samples without the candidate modulator, indicates that the candidate modulator inhibits the GPCRx11 :angiopeptin interaction. Alternatively, FRET technology can be used, according to the invention to detect the binding of a candidate molecule to a G-protein coupled receptor for which the natural ligand is not yet known (i.e., GPCRx2, 5, 6, 7, 9, 13, 14, 16, 17, 18, 19, and 20). In this embodiment, the G-protein coupled receptor and the candidate molecule are labeled with two members of a FRET pair. Thus, binding of the receptor by the candidate molecule may be detected and quantitated as described above by changes in the fluorescence emission.

A variation on FRET uses fluorescence quenching to monitor molecular interactions. One molecule in the interacting pair can be labeled with a fluorophore, and the other with a molecule that quenches the fluorescence of the fluorophore when brought into close apposition with it. A change in fluorescence upon excitation is indicative of a change in the association of the molecules tagged with the fluorophore:quencher pair. Generally, an increase in fluorescence of the labeled GPCRx11 polypeptide is indicative that the angiopeptin molecule bearing the quencher has been displaced. For quenching assays, a 10% or greater increase in the intensity of fluorescent emission in samples containing a candidate modulator, relative to samples without the candidate modulator, indicates that the candidate modulator inhibits GPCRx11 :angiopeptin interaction.

In addition to the surface plasmon resonance and FRET methods, fluorescence polarization measurement is useful to quantitate binding. The fluorescence polarization value for a fluorescently-tagged molecule depends on the rotational correlation time or tumbling rate. Complexes, such as those formed by GPCRx11 associating with a fluorescently labeled angiopeptin, have higher polarization values than uncomplexed, labeled angiopeptin. The inclusion of a candidate inhibitor of the GPCRx11 :angiopeptin interaction results in a decrease in fluorescence polarization, relative to a mixture without the candidate inhibitor, if the candidate inhibitor disrupts or inhibits the interaction of GPCRx11 with angiopeptin. Fluorescence polarization is well suited for the identification of small molecules that disrupt the formation of receptor:ligand complexes. A decrease of 10% or more in fluorescence polarization in samples containing a candidate modulator, relative to fluorescence polarization in a sample lacking the candidate modulator, indicates that the candidate modulator inhibits GPCRx11:angiopeptin interaction.

Another alternative for monitoring GPCRx11 angiopeptin interactions uses a biosensor assay. ICS biosensors have been described in the art (Australian Membrane Biotechnology Research Institute; Cornell B et al., Nature (1997), 387: 580). In this technology, the association of GPCRx11 and its ligand is coupled to the closing of gramacidin-facilitated ion channels in suspended membrane bilayers and thus to a measurable change in the admittance (similar to impedance) of the biosensor. This approach is linear over six orders of magnitude of admittance change and is ideally suited for large scale, high throughput screening of small molecule combinatorial libraries. A 10% or greater change (increase or decrease) in admittance in a sample containing a candidate modulator, relative to the admittance of a sample lacking the candidate modulator, indicates that the candidate modulator inhibits the interaction of GPCRx11 and angiopeptin. It is important to note that in assays testing the interaction of GPCRx11 with angiopeptin, it is possible that a modulator of the interaction need not necessarily interact directly with the domain(s) of the proteins that physically interact with angiopeptin. It is also possible that a modulator will interact at a location removed from the site of interaction and cause, for example, a conformational change in the GPCRx11 polypeptide. Modulators (inhibitors or agonists) that act in this manner are nonetheless of interest as agents to modulate the activity of GPCRx11.

It should be understood that any of the binding assays described herein can be performed with a non-angiopeptin ligand (for example, agonist, antagonist, etc.) of GPCRx11, or one or more of the other G-protein coupled receptors described herein, e.g., a small molecule identified as described herein or angiopeptin analogues including but not limited to any of the angiopeptin analogues, a natural or synthetic peptide, a polypeptide, an antibody or antigen-binding fragment thereof, a lipid, a carbohydrate, and a small organic molecule.

Any of the binding assays described can be used to determine the presence of an agent in a sample, e.g., a tissue sample, that binds to the G-protein coupled receptor molecules described herein, or that affects the binding of angiopeptin to the GPCRx11. To do so, for example, GPCRx11 polypeptide is reacted with angiopeptin or another ligand in the presence or absence of the sample, and angiopeptin or ligand binding is measured as appropriate for the binding assay being used. A decrease of 10% or more in the binding of angiopeptin or other ligand indicates that the sample contains an agent that modulates angiopeptin or ligand binding to the receptor polypeptide. This method could also be adapted by one of skill in the art to determine the presence in a sample of an agent which binds to one or more of the receptors disclosed herein for which a natural ligand is not known.

Functional Assays of Receptor Activity

i. GTPase/GTP Binding Assays

For GPCRs such as those disclosed herein, a measure of receptor activity is the binding of GTP by cell membranes containing receptors. In the method described by Traynor and Nahorski, 1995, Mol. Pharmacol. 47: 848-854, incorporated herein by reference, one essentially measures G-protein coupling to membranes by detecting the binding of labeled GTP. For GTP binding assays, membranes isolated from cells expressing the receptor are incubated in a buffer containing 20 mM HEPES, pH 7.4, 100 mM NaCl, and 10 mM MgCl2, 80 pM ³⁵S-GTPγS and 3 μM GDP. The assay mixture is incubated for 60 minutes at 30° C., after which unbound labeled GTP is removed by filtration onto GF/B filters. Bound, labeled GTP is measured by liquid scintillation counting. This method can thus be used to identify a potential ligand for one or more of GPCRx2, 5, 6, 7, 9, 14, 16, 17, 18, 19, or 20. In addition such an assay can be used to assay for modulation of angiopeptin-induced GPCRx11 activity. Briefly, membranes prepared from cells expressing a GPCRx11 polypeptide are mixed with angiopeptin, and the GTP binding assay is performed in the presence and absence of a candidate modulator of GPCRx11 activity. An increase of 10% or more in labeled GTP binding as measured by scintillation counting in an assay of this kind containing a candidate modulator, relative to an assay without the modulator, indicates that the candidate modulator inhibits GPCRx11 activity. A similar GTP-binding assay can be performed without angiopeptin to identify compounds that act as agonists. In this case, angiopeptin-stimulated GTP binding is used as a standard. A compound is considered an agonist if it induces at least 50% of the level of GTP binding induced by angiopeptin when the compound is present at 1 μM or less, and preferably will induce a level the same as or higher than that induced by angiopeptin.

In an alternate embodiment, GTPase activity can be measured by incubating the membranes containing a G-protein coupled receptor polypeptide with γ³²P-GTP. Active GTPase will release the label as inorganic phosphate, which is detected by separation of free inorganic phosphate in a 5% suspension of activated charcoal in 20 mM H₃PO₄, followed by scintillation counting. Controls include assays using membranes isolated from cells not expressing the G-protein coupled receptor (mock-transfected), in order to exclude possible non-specific effects of the candidate compound. An increase in GTPase activity of at least 10% as measured by the GTPase assay is indicative of activation of the receptor. This assay can thus be used to identify candidate ligands of the G-protein coupled receptors described herein

In order to assay for the effect of a candidate modulator on GPCRx11-regulated GTPase activity, membrane samples are incubated with angiopeptin, with and without the modulator, followed by the GTPase assay. A change (increase or decrease) of 10% or more in the level of GTP binding or GTPase activity relative to samples without modulator is indicative of GPCRx11 modulation by a candidate modulator.

ii. Downstream Pathway Activation Assays

a. Calcium flux—The Aequorin-based Assay

The aequorin assay takes advantage of the responsiveness of mitochondrial apoaequorin to intracellular calcium release induced by the activation of GPCRs (Stables et al., 1997, Anal. Biochem. 252:115-126; Detheux et al., 2000, J. Exp. Med., 192 1501-1508; both of which are incorporated herein by reference). Briefly, GPCR-expressing clones are transfected to coexpress mitochondrial apoaequorin and Gα16. Cells are incubated with 5 μM Coelenterazine H (Molecular Probes) for 4 hours at room temperature, washed in DMEM-F12 culture medium and resuspended at a concentration of 0.5×10⁶ cells/ml. Cells are then mixed with test agonist, or ligand molecules and light emission by the aequorin is recorded with a luminometer for 30 sec. Results are expressed as Relative Light Units (RLU). Controls include assays using membranes isolated from cells not expressing GPCRs (mock transfected), in order to exclude possible non-specific effects of the candidate compound.

Aequorin activity or intracellular calcium levels are “changed” if light intensity increases or decreases by 10% or more in a sample of cells, expressing a GPCR polypeptide and treated with a candidate modulator, relative to a sample of cells expressing the GPCR polypeptide but not treated with the candidate modulator or relative to a sample of cells not expressing the GPCR polypeptide (mock-transfected cells) but treated with the candidate modulator.

When performed in the absence of angiopeptin, the assay can be used to identify an agonist of GPCRx11 activity. When the assay is performed in the presence of angiopeptin, it can be used to assay for an antagonist of GPCRx11 activity.

b. Adenylate Cyclase Assay

Assays for adenylate cyclase activity are described by Kenimer & Nirenberg, 1981, Mol. Pharmacol. 20: 585-591, incorporated herein by reference. That assay is a modification of the assay taught by Solomon et al., 1974, Anal. Biochem. 58: 541-548, also incorporated herein by reference. Briefly, 100 μl reactions contain 50 mM Tris-Hcl (pH 7.5), 5 mM MgCl₂, 20 mM creatine phosphate (disodium salt), 10 units (71 μg of protein) of creatine phosphokinase, 1 mM α-³²P-ATP (tetrasodium salt, 2 μCi), 0.5 mM cyclic AMP, G-³H-labeled cyclic AMP (approximately 10,000 cpm), 0.5 mM Ro20-1724, 0.25% ethanol, and 50-200 μg of protein homogenate to be tested (i.e., homogenate from cells expressing or not expressing a GPCR polypeptide, treated or not treated with a ligand, with or without a candidate modulator). Reaction mixtures are generally incubated at 37° C. for 60 minutes. Following incubation, reaction mixtures are deproteinized by the addition of 0.9 ml of cold 6% trichloroacetic acid. Tubes are centrifuged at 1800×g for 20 minutes and each supernatant solution is added to a Dowex AG50W-X4 column. The cAMP fraction from the column is eluted with 4 ml of 0.1 mM imidazole-HCl (pH 7.5) into a counting vial. Assays should be performed in triplicate. Control reactions should also be performed using protein homogenate from cells that do not express a GPCR polypeptide.

According to the invention, adenylate cyclase activity is “changed” if it increases or decreases by 10% or more in a sample taken from cells treated with a candidate modulator of GPCR activity, relative to a similar sample of cells not treated with the candidate modulator or relative to a sample of cells not expressing the GPCR polypeptide (mock-transfected cells) but treated with the candidate modulator.

c. cAMP Assay

Intracellular or extracellular cAMP is measured using a cAMP radioimmunoassay (RIA) or cAMP binding protein according to methods widely known in the art. For example, Horton & Baxendale, 1995, Methods Mol. Biol. 41: 91-105, which is incorporated herein by reference, describes an RIA for cAMP.

A number of kits for the measurement of cAMP are commercially available, such as the High Efficiency Fluorescence Polarization-based homogeneous assay marketed by LJL Biosystems and NEN Life Science Products. Control reactions should be performed using extracts of mock-transfected cells to exclude possible non-specific effects of some candidate modulators.

The level of cAMP is “changed” if the level of cAMP detected in cells, expressing a GPCR polypeptide and treated with a candidate modulator of GPCR activity (or in extracts of such cells), using the RIA-based assay of Horton & Baxendale, 1995, supra, increases or decreases by at least 10% relative to the cAMP level in similar cells not treated with the candidate modulator.

d. Phospholipid breakdown, DAG production and Inositol Triphosphate levels

Receptors that activate the breakdown of phospholipids can be monitored for changes due to the activity of known or suspected modulators of GPCRs according to the invention, preferably GPCRx11, by monitoring phospholipid breakdown, and the resulting production of second messengers DAG and/or inositol triphosphate (IP3). Methods of detecting each of these are described in Phospholipid Signaling Protocols, edited by Ian M. Bird. Totowa, N.J., Humana Press, 1998, which is incorporated herein by reference. See also Rudolph et al., 1999, J. Biol. Chem. 274: 11824-11831, incorporated herein by reference, which also describes an assay for phosphatidylinositol breakdown. Assays should be performed using cells or extracts of cells expressing GPCR polypeptide, treated or not treated with ligand with or without a candidate modulator. Control reactions should be performed using mock-transfected cells, or extracts from them in order to exclude possible non-specific effects of some candidate modulators.

According to the invention, phosphatidylinositol breakdown, and diacylglycerol and/or inositol triphosphate levels are “changed” if they increase or decrease by at least 10% in a sample from cells expressing a GPCR polypeptide and treated with a candidate modulator, relative to the level observed in a sample from cells expressing a GPCR polypeptide that is not treated with the candidate modulator.

e. PKC Activation Assays

Growth factor receptor tyrosine kinases can signal via a pathway involving activation of Protein Kinase C (PKC), which is a family of phospholipid- and calcium-activated protein kinases. PKC activation ultimately results in the transcription of an array of proto-oncogene transcription factor-encoding genes, including c-fos, c-myc and c-jun, proteases, protease inhibitors, including collagenase type I and plasminogen activator inhibitor, and adhesion molecules, including intracellular adhesion molecule I (ICAM I). Assays designed to detect increases in gene products induced by PKC can be used to monitor PKC activation and thereby receptor activity. In addition, the activity of receptors that signal via PKC can be monitored through the use of reporter gene constructs driven by the control sequences of genes activated by PKC activation. This type of reporter gene-based assay is discussed in more detail below.

For a more direct measure of PKC activity, the method of Kikkawa et al., 1982, J. Biol. Chem. 257: 13341, incorporated herein by reference, can be used. This assay measures phosphorylation of a PKC substrate peptide, which is subsequently separated by binding to phosphocellulose paper. This PKC assay system can be used to measure activity of purified kinase, or the activity in crude cellular extracts. Protein kinase C sample can be diluted in 20 mM HEPES/2 mM DTT immediately prior to assay.

The substrate for the assay is the peptide Ac-FKKSFKL-NH2, derived from the myristoylated alanine-rich protein kinase C substrate protein (MARCKS). The Kₘ of the enzyme for this peptide is approximately 50 μM. Other basic, protein kinase C-selective peptides known in the art can also be used, at a concentration of at least 2-3 times their Kₘ. Cofactors required for the assay include calcium, magnesium, ATP, phosphatidylserine and diacylglycerol. Depending upon the intent of the user, the assay can be performed to determine the amount of PKC present (activating conditions) or the amount of active PKC present (non-activating conditions). For most purposes according to the invention, non-activating conditions will be used, such that the PKC, that is active in the sample when it is isolated, is measured, rather than measuring the PKC that can be activated. For non-activating conditions, calcium is omitted from the assay in favor of EGTA.

The assay is performed in a mixture containing 20 mM HEPES, pH 7.4, 1-2 mM DTT, 5 mM MgCl₂, 100 μM ATP, ˜1 μCi γ-³²P-ATP, 100 μg/ml peptide substrate (˜100 μM), 140 μM/ 3.8 μM phosphatidylserine/diacylglycerol membranes, and 100 μM calcium (or 500 μM EGTA). 48 μl of sample, diluted in 20 mM HEPES, pH 7.4, 2 mM DTT is used in a final reaction volume of 80 μl. Reactions are performed at 30° C. for 5-10 minutes, followed by addition of 25 μl of 100 mM ATP, 100 mM EDTA, pH 8.0, which stops the reactions.

After the reaction is stopped, a portion (85 μl) of each reaction is spotted onto a Whatman P81 cellulose phosphate filter, followed by washes: four times 500 ml in 0.4% phosphoric acid, (5-10 min per wash); and a final wash in 500 ml 95% EtOH, for 2-5 min. Bound radioactivity is measured by scintillation counting. Specific activity (cpm/nmol) of the labeled ATP is determined by spotting a sample of the reaction onto P81 paper and counting without washing. Units of PKC activity, defined as nmol phosphate transferred per min, are calculated as follows:

The activity, in UNITS (nmol/min) is:

$$The activity , in UNITS ( nmol \text{/} min ) is \text{:} = \frac{( cpm on paper ) \times ( 105 µ 1 total / 85 µ1 spotted )}{( assay time , min ) ( specific activity of ATP cpm \text{/} nmol )} .$$

An alternative assay can be performed using a Protein Kinase C Assay Kit sold by PanVera (Cat. #P2747).

Assays are performed on extracts from cells expressing a GPCR polypeptide, treated or not treated with ligand (if it is known, or with a candidate ligand if the natural ligand is not known) with or without a candidate modulator. Control reactions should be performed using mock-transfected cells, or extracts from them in order to exclude possible non-specific effects of some candidate modulators.

According to the invention, PKC activity is “changed” by a candidate modulator when the units of PKC measured by either assay described above increase or decrease by at least 10%, in extracts from cells expressing GPCR and treated with a candidate modulator, relative to a reaction performed on a similar sample from cells not treated with a candidate modulator.

f. Kinase Assays

MAP kinase activity can be assayed using any of several kits available commercially, for example, the p38 MAP Kinase assay kit sold by New England Biolabs (Cat #9820) or the FlashPlate™ MAP Kinase assays sold by Perkin-Elmer Life Sciences.

MAP Kinase activity is “changed” if the level of activity is increased or decreased by 10% or more in a sample from cells, expressing a GPCR polypeptide, treated with a candidate modulator relative to MAP kinase activity in a sample from similar cells not treated with the candidate modulator.

Direct assays for tyrosine kinase activity using known synthetic or natural tyrosine kinase substrates and labeled phosphate are well known, as are similar assays for other types of kinases (e.g., Ser/Thr kinases). Kinase assays can be performed with both purified kinases and crude extracts prepared from cells expressing a GPCR polypeptide, treated with or without ligand (such as angiopeptin for GPCRx11, or a candidate ligand in the case of a GPCR for which the natural ligand is not known), with or without a candidate modulator. Control reactions should be performed using mock-transfected cells, or extracts from them in order to exclude possible non-specific effects of some candidate modulators. Substrates can be either full-length protein or synthetic peptides representing the substrate. Pinna & Ruzzene (1996, Biochem. Biophys. Acta 1314: 191-225, incorporated herein by reference) list a number of phosphorylation substrate sites useful for detecting kinase activities. A number of kinase substrate peptides are commercially available. One that is particularly useful is the “Src-related peptide,” RRLIEDAEYAARG (SEQ ID NO: 29; available from Sigma #A7433), which is a substrate for many receptor and nonreceptor tyrosine kinases. Because the assay described below requires binding of peptide substrates to filters, the peptide substrates should have a net positive charge to facilitate binding. Generally, peptide substrates should have at least 2 basic residues and a free amino terminus. Reactions generally use a peptide concentration of 0.7-1.5 mM.

Assays are generally carried out in a 25 μl volume comprising 5 μl of 5×kinase buffer (5 mg/mL BSA, 150 mM Tris-Cl (pH 7.5), 100 mM MgCl₂; depending upon the exact kinase assayed for, MnCl₂ can be used in place of or in addition to the MgCl₂), 5 μl of 1.0 mM ATP (0.2 mM final concentration), γ-32P-ATP (100-500 cpm/pmol), 3 μl of 10 mM peptide substrate (1.2 mM final concentration), cell extract containing kinase to be tested (cell extracts used for kinase assays should contain a phosphatase inhibitor (e.g. 0.1-1 mM sodium orthovanadate)), and H₂O to 25 μl. Reactions are performed at 30° C., and are initiated by the addition of the cell extract.

Kinase reactions are performed for 30 seconds to about 30 minutes, followed by the addition of 45 μl of ice-cold 10% trichloroacetic acid (TCA). Samples are spun for 2 minutes in a microcentrifuge, and 35 μl of the supernatant is spotted onto Whatman P81 cellulose phosphate filter circles. The filters are washed three times with 500 ml cold 0.5% phosphoric acid, followed by one wash with 200 ml of acetone at room temperature for 5 minutes. Filters are dried and incorporated ³²P is measured by scintillation counting. The specific activity of ATP in the kinase reaction (e.g., in cpm/pmol) is determined by spotting a small sample (2-5 μl) of the reaction onto a P81 filter circle and counting directly, without washing. Counts per minute obtained in the kinase reaction (minus blank) are then divided by the specific activity to determine the moles of phosphate transferred in the reaction.

Tyrosine kinase activity is “changed” if the level of kinase activity is increased or decreased by 10% or more in a sample from cells, expressing a GPCR polypeptide, treated with a candidate modulator relative to kinase activity in a sample from similar cells not treated with the candidate modulator.

g. Transcriptional Reporters for Downstream Pathway Activation

The intracellular signal initiated by binding of an agonist to a receptor, e.g., GPCRx11, sets in motion a cascade of intracellular events, the ultimate consequence of which is a rapid and detectable change in the transcription or translation of one or more genes. The activity of the receptor can therefore be monitored by detecting the expression of a reporter gene driven by control sequences responsive to receptor activation.

As used herein “promoter” refers to the transcriptional control elements necessary for receptor-mediated regulation of gene expression, including not only the basal promoter, but also any enhancers or transcription-factor binding sites necessary for receptor-regulated expression. By selecting promoters that are responsive to the intracellular signals resulting from agonist binding, and operatively linking the selected promoters to reporter genes whose transcription, translation or ultimate activity is readily detectable and measurable, the transcription based reporter assay provides a rapid indication of whether a given receptor is activated.

Reporter genes such as luciferase, CAT, GFP, β-lactamase or β-galactosidase are well known in the art, as are assays for the detection of their products.

Genes particularly well suited for monitoring receptor activity are the “immediate early” genes, which are rapidly induced, generally within minutes of contact between the receptor and the effector protein or ligand. The induction of immediate early gene transcription does not require the synthesis of new regulatory proteins. In addition to rapid responsiveness to ligand binding, characteristics of preferred genes useful for making reporter constructs include: low or undetectable expression in quiescent cells; induction that is transient and independent of new protein synthesis; subsequent shut-off of transcription requires new protein synthesis; and mRNAs transcribed from these genes have a short half-life. It is preferred, but not necessary that a transcriptional control element have all of these properties for it to be useful.

An example of a gene that is responsive to a number of different stimuli is the c-fos proto-oncogene. The c-fos gene is activated in a protein-synthesis-independent manner by growth factors, hormones, differentiation-specific agents, stress, and other known inducers of cell surface proteins. The induction of c-fos expression is extremely rapid, often occurring within minutes of receptor stimulation. This characteristic makes the c-fos regulatory regions particularly attractive for use as a reporter of receptor activation.

The c-fos regulatory elements include (see, Verma et al., 1987, Cell 51: 513-514): a TATA box that is required for transcription initiation; two upstream elements for basal transcription, and an enhancer, which includes an element with dyad symmetry and which is required for induction by TPA, serum, EGF, and PMA.

The 20 bp c-fos transcriptional enhancer element located between −317 and −298 bp upstream from the c-fos mRNA cap site, is essential for serum induction in serum starved NIH 3T3 cells. One of the two upstream elements is located at −63 to −57 and it resembles the consensus sequence for cAMP regulation.

The transcription factor CREB (cyclic AMP responsive element binding protein) is, as the name implies, responsive to levels of intracellular cAMP. Therefore, the activation of a receptor that signals via modulation of cAMP levels can be monitored by detecting either the binding of the transcription factor, or the expression of a reporter gene linked to a CREB-binding element (termed the CRE, or cAMP response element). The DNA sequence of the CRE is TGACGTCA. Reporter constructs responsive to CREB binding activity are described in U.S. Pat. No. 5,919,649.

Other promoters and transcriptional control elements, in addition to the c-fos elements and CREB-responsive constructs, include the vasoactive intestinal peptide (VIP) gene promoter (cAMP responsive; Fink et al., 1988, Proc. Natl. Acad. Sci. 85:6662-6666); the somatostatin gene promoter (cAMP responsive; Montminy et al., 1986, Proc. Natl. Acad. Sci. 8.3:6682-6686); the proenkephalin promoter (responsive to cAMP, nicotinic agonists, and phorbol esters; Comb et al., 1986, Nature 323:353-356); the phosphoenolpyruvate carboxy-kinase (PEPCK) gene promoter (cAMP responsive; Short et al., 1986, J. Biol. Chem. 261:9721-9726).

Additional examples of transcriptional control elements that are responsive to changes in GPCR activity include, but are not limited to those responsive to the AP-1 transcription factor and those responsive to NF-κB activity. The consensus AP-1 binding site is the palindrome TGA(C/G)TCA (Lee et al., 1987, Nature 325: 368-372; Lee et al., 1987, Cell 49: 741-752). The AP-1 site is also responsible for mediating induction by tumor promoters such as the phorbol ester 12-O-tetradecanoylphorbol-β-acetate (TPA), and are therefore sometimes also referred to as a TRE, for TPA-response element. AP-1 activates numerous genes that are involved in the early response of cells to growth stimuli. Examples of AP-I-responsive genes include, but are not limited to the genes for Fos and Jun (which proteins themselves make up AP-1 activity), Fos-related antigens (Fra) 1 and 2, IκBα, ornithine decarboxylase, and annexins I and II.

The NF-κB binding element has the consensus sequence GGGGACTTTCC (SEQ ID NO: 30). A large number of genes have been identified as NF-κB responsive, and their control elements can be linked to a reporter gene to monitor GPCR activity. A small sample of the genes responsive to NF-κB includes those encoding IL-1β (Hiscott et al., 1993, Mol. Cell. Biol. 13: 6231-6240), TNF-α: (Shakhov et al., 1990, J. Exp. Med. 171: 35-47), CCR5 (Liu et al., 1998, AIDS Res. Hum. Retroviruses 14: 1509-1519), P-selection (Pan & McEver, 1995, J. Biol. Chem. 270:23077-23083), Fas ligand (Matsui et al., 1998, J. Immunol. 161: 3469-3473), GM-CSF (Schreck & Baeuerle, 1990, Mol. Cell. Biol. 10: 1281-1286) and IKBXo (Haskill et al., 1991, Cell 65: 1281-1289). Each of these references is incorporated herein by reference. Vectors encoding NF-κB-responsive reporters are also known in the art or can be readily made by one of skill in the art using, for example, synthetic NF-κB elements and a minimal promoter, or using the NF-κB-responsive sequences of a gene known to be subject to NF-κB regulation. Further, NF-κB responsive reporter constructs are commercially available from, for example, CLONTECH.

A given promoter construct may be tested by exposing GPCRx11-expressing cells, transfected with the construct, to angiopeptin. An increase of at least two-fold in the expression of reporter in response to angiopeptin indicates that the reporter is an indicator of GPCRx11 activity.

In order to assay GPCR activity with a transcriptional reporter construct, cells that stably express a GPCR polypeptide are stably transfected with the reporter construct. To screen for agonists, the cells are left untreated, exposed to candidate modulators, or exposed to angiopeptin (or a candidate ligand in the case of a GPCR for which the natural ligand is not known), and expression of the reporter is measured. The angiopeptin-treated cultures serve as a standard for the level of transcription induced by a known agonist. An increase of at least 50% in reporter expression in the presence of a candidate modulator indicates that the candidate is a modulator of GPCRx11 activity. An agonist will induce at least as much, and preferably the same amount or greater reporter expression than angiopeptin alone. This approach can also be used to screen for inverse agonists where cells express a GPCRx11 polypeptide at levels such that there is an elevated basal activity of the reporter in the absence of propionate or another agonist. A decrease in reporter activity of 10% or more in the presence of a candidate modulator, relative to its absence, indicates that the compound is an inverse agonist.

To screen for antagonists, the cells expressing GPCRx11 and carrying the reporter construct are exposed to angiopeptin (or another agonist) in the presence and absence of candidate modulator. A decrease of 10% or more in reporter expression in the presence of candidate modulator, relative to the absence of the candidate modulator, indicates that the candidate is a modulator of GPCRx11 activity.

Controls for transcription assays include cells not expressing GPCRs but carrying the reporter construct, as well as cells with a promoterless reporter construct. Compounds that are identified as modulators of GPCRx11-regulated transcription should also be analyzed to determine whether they affect transcription driven by other regulatory sequences and by other receptors, in order to determine the specificity and spectrum of their activity.

The transcriptional reporter assay, and most cell-based assays, are well suited for screening expression libraries for proteins for those that modulate GPCRx11 activity. The libraries can be, for example, cDNA libraries from natural sources, e.g., plants, animals, bacteria, etc., or they can be libraries expressing randomly or systematically mutated variants of one or more polypeptides. Genomic libraries in viral vectors can also be used to express the mRNA content of one cell or tissue in the different libraries used for screening of GPCRx11.

h. Inositol Phosphates (IP) Measurement

Cells of the invention, for example, CHO-K1 cells, are labelled for 24 hours with 10 μCi/ml [³H] inositol in inositol free DMEM containing 5% FCS, antibiotics, amphotericin, sodium pyruvate and 400 μg/ml G418. Cells are incubated for 2 h in Krebs-Ringer Hepes (KRH) buffer of the following composition (124 mM NaCl, 5 mM KCl, 1.25 mM MgSO₄, 1.45 mM CaCl₂, 1.25 mM KH₂PO₄, 25 mM Hepes (pH:7.4) and 8 mM glucose). The cells are then challenged with angiopeptin for 30 min. The incubation is stopped by the addition of an ice cold 3% perchloric acid solution. IP are extracted and separated on Dowex columns as previously described (25).

i. Membrane Polarization Assays for Measurement of Receptor Activity

Electrophysiological measurements of receptor activity may be performed using standard patch-clamp techniques, as described by Hoo et al. (1994, Receptors and Channels 2: 327), and summarized as follows.

Electrophysiological recordings are performed in a standard extracellular solution composed of 140 mM NaCl, 5.4 mM KCl, 1.0 mM MgCl₂, 1.3 mM CaCl₂, 5.0 mM HEPES and glucose to an osmolarity of 300 mOsm and pH adjusted to 7.2 with 1 mM NaOH.

For ion permeability studies, two other recording solutions are used, including a low calcium solution (140 mM NaCl, 1.0 mM MgCl2, 5.0 mM HEPES (pH 7.2 with NaOH)), and a low sodium solution (110 mM CaCl2, 1.0 mM MgCl2, 5.0 mM HEPES (pH to 7.2 with Ca(OH)₂).

Electrodes are constructed from thin-walled borosilicate glass (WPI Instruments), pulled to a fine point (1-2 μm in width) and filled with an intracellular solution composed of 140 mM CsCl, 1.0 mM MgCl2, 10 mM EGTA, 10 mM HEPES with pH adjusted to 7.2 with Cs(OH)2 and an osmolarity of 300 mOsm.

Whole cell voltage clamp recordings are carried out using an Axopatch 1B amplifier (Axon Instruments) or its equivalent. Agonists and antagonists are rapidly perfused over the cells through a multibarrel array of square glass tubes, the position of which is adjusted using a piezomotor under computer control. With this system it is possible to rapidly exchange solutions flowing over the cell and thus carry out extensive studies of receptor pharmacology.

Such electrophysiological assays may be performed on cells which express one or more GPCRs according to the invention, wherein a change in membrane potential or the observation of inward or outward currents upon application of a ligand (or a candidate ligand in the case of GPCRs for which the natural ligand is not known), a candidate modulator, or a ligand plus a candidate modulator is indicative of a change in receptor activity.

GPCRx11 Assay

The invention provides for an assay for detecting the activity of a receptor, preferably GPCRx11 of the invention in a sample. For example, GPCRx11 activity can be measured in a sample comprising a cell or a cell membrane that expresses GPCRx11. As above, angiopeptin is used as an example in this section. The assay is performed by incubating the sample in the presence or absence of angiopeptin and carrying out a second messenger assay, as described above. The results of the second messenger assay performed in the presence or absence of angiopeptin are compared to determine if the GPCRx11 receptor is active. An increase of 10% or more in the detected level of a given second messenger, as defined herein, in the presence of angiopeptin relative to the amount detected in an assay performed in the absence of angiopeptin is indicative of GPCRx11 activity.

Any of the assays of receptor activity, including but not limited to the GTP-binding, GTPase, adenylate cyclase, cAMP, phospholipid-breakdown, diacylglycerol, inositol triphosphate, arachidonic acid release (see below), PKC, kinase and transcriptional reporter assays, can be used to determine the presence of an agent in a sample, e.g., a tissue sample, that affects the activity of the GPCRx11 receptor molecule. To do so, GPCRx11 polypeptide is assayed for activity in the presence and absence of the sample or an extract of the sample. An increase in GPCRx11 activity in the presence of the sample or extract relative to the absence of the sample indicates that the sample contains an agonist of the receptor activity. A decrease in receptor activity in the presence of propionate or another agonist and the sample, relative to receptor activity in the presence of propionate alone, indicates that the sample contains an antagonist of GPCRxl activity. If desired, samples can then be fractionated and further tested to isolate or purify the agonist or antagonist. The amount of increase or decrease in measured activity necessary for a sample to be said to contain a modulator depends upon the type of assay used. Generally, a 10% or greater change (increase or decrease) relative to an assay performed in the absence of a sample indicates the presence of a modulator in the sample. One exception is the transcriptional reporter assay, in which at least a two-fold increase or 10% decrease in signal is necessary for a sample to be said to contain a modulator. It is preferred that an agonist stimulates at least 50%, and preferably 75% or 100% or more, e.g., 2-fold, 5-fold, 10-fold or greater receptor activation than with propionate alone.

Other functional assays include, for example, microphysiometer or biosensor assays (see Hafner, 2000, Biosens. Bioelectron. 15:149-158, incorporated herein by reference). The intracellular level of arachinoid acid can also be determined as described in Gijon et al., 2000, J. Biol. Chem., 275:20146-20156.

II. Diagnostic Assays

Signaling through GPCRs is instrumental in the pathology of a large number of diseases and disorders. GPCRx11 is expressed in reproductive cells as well as cells of immune organs such as the thymus. The other GPCRs disclosed herein have been observed, as described below, to be expressed in cells of the lymphocyte lineages, brain and spinal cord, spleen, stomach, lung as well as leukemic cells, can have a role in immune processes, cancer, neurological diseases, gasteroenterological diseases, and associated disorders or diseases.

The expression pattern of GPCRs and the knowledge with respect to disorders generally mediated by GPCRs suggests that the G-protein coupled receptors of the present invention can be involved in disturbances of cell migration, cancer, development of tumors and tumor metastasis, inflammatory and neoplastic processes, wound and bone healing and dysfunction of regulatory growth functions, diabetes, obesity, anorexia, bulimia, acute heart failure, hypotension, hypertension, urinary retention, osteoporosis, angina pectoris, myocardial infarction, restenosis, atherosclerosis, thrombosis and other cardiovascular diseases, autoimmune and inflammatory diseases, diseases characterized by excessive smooth muscle cell proliferation, aneurysms, diseases characterized by loss of smooth muscle cells or reduced smooth muscle cell proliferation, stroke, ischemia, ulcers, allergies, benign prostatic hypertrophy, migraine, vomiting, psychotic and neurological disorders, including anxiety, schizophrenia, manic depression, depression, delirium, dementia and severe mental retardation, degenerative diseases, neurodegenerative diseases such as Alzheimer's disease or Parkinson's disease, and dyskinasias, such as Huntington's disease or Gilles de la Tourett's syndrome and other related diseases including thrombosis and other cardiovascular diseases, autoimmune and inflammatory diseases.

The interaction of GPCRx11 with angiopeptin can be used as the basis of assays for the diagnosis or monitoring of diseases, disorders or processes involving GPCRx11 signaling. Diagnostic assays for GPCR and GPCRx11-related diseases or disorders can have several different forms. First, diagnostic assays can measure the amount of GPCR polypeptides, mRNA or ligand in a sample of tissue. Assays that measure the amount of mRNA encoding GPCR polypeptides also fit into this category. Second, assays can evaluate the qualities of the receptor or the ligand. For example, assays that determine whether an individual expresses a mutant or variant form of GPCRx11 can be used diagnostically. Third, assays that measure one or more activities of GPCRx11 polypeptide can be used diagnostically.

A. Assays that Measure the Amount of GPCR Polypeptide

In one embodiment, the present invention provides a diagnostic assay to determine whether the GPCRs of the present invention are expressed in a given cell population. Protein expression in cells may be determined using any technique known to those of skill in the art including but not limited to Western Blot, ELISA, immunoprecipitation, immunohistochemistry, and the like.

For example, the GPCR of the present invention may be detected in a cell by immunohistochemistry using an antibody which specifically binds to the GPCR of the invention or an immunogenic portion thereof.

i. Generation of Antibodies

Antibodies that bind to the protein products encoded by a polynucleotide comprising a sequence of the invention are useful for protein purification, for the diagnosis and treatment of various diseases and for drug screening and drug design methods useful for identifying and developing compounds to be used in the treatment of various diseases. The term “antibody” is meant to encompass constructions using the binding (variable) region of such an antibody, and other antibody modifications. Thus, an antibody useful in the invention may comprise a whole antibody, an antibody fragment, a polyfunctional antibody aggregate, or in general a substance comprising one or more specific binding sites from an antibody. The antibody fragment may be a fragment such as an Fv, Fab or F(ab′)₂ fragment or a derivative thereof, such as a single chain Fv fragment. The antibody or antibody fragment may be non-recombinant, recombinant or humanized. The antibody may be of an immunoglobulin isotype, e.g., IgG, IgM, and so forth. In addition, an aggregate, polymer, derivative and conjugate of an immunoglobulin or a fragment thereof can be used where appropriate. Neutralizing antibodies are especially useful according to the invention for diagnostics, therapeutics and methods of drug screening and drug design.

Although a protein product (or fragment or oligopeptide thereof) derived from a polynucleotide comprising a sequence of the invention that is useful for the production of antibodies does not require biological activity, it must be antigenic. Peptides used to induce specific antibodies may have an amino acid sequence consisting of at least five amino acids and more conveniently at least ten amino acids. It is advantageous for such peptides to be identical to a region of the natural protein and they may contain the entire amino acid sequence of a small, naturally occurring molecule. Short stretches of amino acids corresponding to the protein product of a candidate gene of the invention may be fused with amino acids from another protein such as keyhole limpet hemocyanin or GST, and antibody will be produced against the chimeric molecule. Procedures well known in the art can be used for the production of antibodies to the protein products derived from the polynucleotide comprising a sequence of the invention.

For the production of antibodies, various hosts including goats, rabbits, rats, mice, etc., may be immunized by injection with the protein products (or any portion, fragment, or oligonucleotide thereof which retains immunogenic properties) of the candidate genes of the invention. Depending on the host species, various adjuvants may be used to increase the immunological response. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful human adjuvants.

To generate polyclonal antibodies, the antigen protein may be conjugated to a conventional carrier in order to increase its immunogenicity, and an antiserum to the peptide-carrier conjugate raised. Coupling of a peptide to a carrier protein and immunizations may be performed as described in Dymecki et al., 1992, J. Biol. Chem., 267:4815. The serum can be titered against protein antigen by ELISA (below) or alternatively by dot or spot blotting (Boersma & Van Leeuwen, 1994, J. Neurosci. Methods, 51:317). A useful serum will react strongly with the appropriate peptides by ELISA, for example, following the procedures of Green et al., 1982, Cell, 28:477.

Techniques for preparing monoclonal antibodies are well known, and monoclonal antibodies may be prepared using a candidate antigen whose level is to be measured or which is to be either inactivated or affinity-purified, preferably bound to a carrier, as described by Arniheiter et al., 1981, Nature, 294:278.

Monoclonal antibodies are typically obtained from hybridoma tissue cultures or from ascites fluid obtained from animals into which the hybridoma tissue was introduced. Monoclonal antibody-producing hybridomas (or polyclonal sera) can be screened for antibody binding to the target protein according to methods known in the art.

ii. Use of Antibodies to Detect a GPCR

A polyclonal or monoclonal antibody or fragment thereof (in subsequent discussions, “antibody” is meant to include all such forms), prepared according to the methods described above and to the references therein included, allows the detection of the presence or absence of the protein encoded by a gene comprising the polynucleotide comprising a sequence of the invention in cells, tissues, or other samples derived from them. Such an antibody preparation will be used as a diagnostic marker to detect the presence or absence of disease associated with the presence (over-, or underabundance relative to non-diseased tissue) or absence of the polypeptide encoded by the novel polynucleotide comprising the sequence disclosed.

Immunological tests rely on the use of either monoclonal or polyclonal antibodies and include enzyme-linked immunoassays (ELISA), immunoblotting and immunoprecipitation (see Voller, 1978, Diagnostic Horizons, 2: 1, Microbiological Associates Quarterly Publication, Walkersville, Md.; Voller et al., 1978, J. Clin. Pathol., 31:507; U.S. Reissue Pat. No. 31,006; UK Patent No. 2,019,408; Butler, 1981, Methods Enzvmol., 73:482; Maggio, E. (ed.), 1980, Enzyme Immunoassay, (CRC Press, Boca Raton, Fla.) or Radioimmunoassays (RIA) (Weintraub, B., Principles of Radioimmunoassays, Seventh Training Course on Radioligand Assay Techniques, The Endocrine Society, March 1986, pp. 1-5, 46-49 and 68-78).

In addition to the methods mentioned above, organized tissues can be examined for the presence or absence of a protein produced by a polynucleotide according to the present invention using immunohistochemistry techniques. Broadly defined, immunohistochemistry is the term for the detection of specific antigens in tissue preparations. The method basically involves the steps of: 1) preparing fixed sections of the tissue of interest, immobilized on microscope slides; 2) incubation of the tissue sections with an antibody preparation specific for the antigen of interest; 3) removal of non-bound antibodies; and 4) detection of antibody-antigen complexes on the tissue sections.

Protocols for immunohistochemistry vary widely, as antigens and their recognition by particular antibody preparations differ dramatically, as do the tissue contexts of the antigen. Thus, different tissues require different methods of processing. For example, tissues may be fixed in paraformaldehyde or another fixative and embedded in paraffin wax or simply frozen prior to sectioning. In addition, the treatment of sectioned tissue will vary according to the antigen and antibody involved and according to the detection method used. Detection typically involves reaction of the bound antibody with a secondary antibody specific for a constant region domain of the antibody which reacts with the experimental antigen target (the so called “primary antibody”), but can alternatively be accomplished by labeling the primary antibody directly, such as with radiolabel, or with a fluorescence or enzyme tag (methods of antibody labeling are described in Harlow & Lane, 1989, Antibodies, Cold Spring Harbor Laboratory, Cold Spring Harbor, N.Y.). When the secondary antibody approach is used, the secondary antibody is typically conjugated to a detection moiety such as a radionuclide, an enzyme (e.g., horseradish peroxidase) or fluorescent tag. Secondary antibodies bearing moieties for detection by an array of different methods, and suitable for the detection of a wide variety of primary antibody types are commercially available. Detailed methods for sample preparation and performance of immunohistochemical analyses are described in Ausubel et al., 1992, supra, pp. 14-22 to 14-29, and in Humason, G. L., 1979, Animal Tissue Techniques, 4th ed. (W. H. Freeman & Co., San Francisco, Calif.). Decisions regarding the use of tissue processing methods and subsequent steps, such as detection, and other considerations, such as appropriate positive and negative controls, depend upon which tissue and antigen are under investigation, but may be made with a limited amount of experimentation by one skilled in the art.

It is contemplated that immunohistochemical methods will allow detection of the polypeptide encoded by the gene sequence comprising the polynucleotide of the invention in the following tissues: muscle, including but not limited to skeletal, cardiac, and smooth muscle; cells and tissues of the circulatory system, including but not limited to those of veins and arteries; cells of the skin; bone and bone forming cells; neuronal cells and tissues of the central nervous system, including but not limited to those of the brain and spine; liver cells, including but not limited to parenchymal and non-parenchymal hepatic cells; cells and tissues of the alimentary tract, including but not limited to esophagus, stomach, large and small intestines and rectum; tissues and cells of the reproductive systems, including but not limited to those of the ovary and testis, uterus, cervix, breast and prostate; cells and tissues of the genitourinary tract, including but not limited to kidney and bladder; cells and tissues of the endocrine system, including but not limited to the adrenal glands, hypothalamus, pituitary gland, and pancreas; immune system components, including but not limited to cells of bone marrow, lymphoid and myeloid lineages, B cells, T cells, NK cells, macrophages, and cells of the spleen; cells and tissues of the pulmonary system and lung; cells of the eye and cells of the auditory system.

It is contemplated that immunohistochemistry, performed as described above, may be used to correlate the presence or absence of a polypeptide encoded by a gene sequence comprising the polynucleotide of the invention with the presence of a disease that is associated with the polypeptide. In order for the antibodies of the invention to be used in such a way, it is necessary to perform immunohistochemical analysis on non-diseased tissues with the same antibody to establish the baseline levels of the polypeptide detected with the antibody. Thus the use of the antibody specific for the protein product of the invention as a diagnostic indicator may comprise the steps of: 1) performing immunohistochemical analysis on cells, tissues, or other samples derived from an individual suffering from, or thought to be suffering from a disease potentially related to the expression of the gene of this invention; and 2) comparing the immunohistochemical signal obtained with that observed in samples derived from non-diseased sources (preferably, but not necessarily derived from non-diseased tissue of the same patient). Presumably, the inappropriate presence (over- or underabundance) or absence of the protein product of the invention in samples derived from diseased tissues, relative to the level of such product in non-diseased tissues indicates that the product of the sequence is involved in or is diagnostic of a disease, or the propensity for a disease.

B. Assays the Determine the Presence of Nucleic Acid Encoding a GPCR

One embodiment of the present invention is the use of the polynucleotide sequence encoding the GPCRs (SEQ ID Nos.: 1, 3, 5, 7, 9, 11, 13, 15, 17, 19, 21, 23, 25, and 27) of the present invention, or a sequence complementary thereto as a probe or probes which allow the detection of DNA or RNA (“target sequence”) corresponding or complementary to that of the disclosed sequence in cells, tissues, or samples derived therefrom. This embodiment will be referred to as a “probe for in situ hybridization analysis.” Thus, the nucleic acid sequences disclosed allows the determination of the presence or absence of the DNA or RNA sequence disclosed in a given cell, tissue, or other sample preparation. In addition, such use allows determination of the cell or tissue-specific expression pattern of the newly identified gene encoded by the disclosed polynucleotide, as well as the levels of expression of its transcript. In turn, knowledge of the patterns and levels of expression of the gene in normal cells or tissues allows comparison with the levels seen in various disease states.

The probe may be DNA, RNA, or modified forms thereof as discussed above, and may be designed to hybridize with the sense or antisense strands of the target sequence, or both.

As embodied as a probe for in situ hybridization analysis, the probe may be of any suitable length and base composition, spanning the whole of or any number of portions of the disclosed sequence or its corresponding genomic sequence. As used herein, “suitable length and base composition” refers to the selection of probe length and base composition such that the probe will hybridize in a specific manner with the target sequence under stringent conditions. As used herein, “stringent conditions” means hybridization will occur only if there is at least 95%, preferably at least 97%, and optimally 100% identity or complementarity between the probe and the sequences it binds. Specific solution compositions and methods for hybridization under stringent conditions are described herein below.

One method of in situ hybridization involves the use of oligonucleotide probes complementary to the desired target nucleic acid sequence. Considerations involved in designing oligonucleotide probes for in situ hybridization analysis are similar to those involved in designing an oligonucleotide probe to screen a library, as discussed above. Oligonucleotides for use in in situ hybridization will generally be between 8 and 100 bases in length, preferably between 8 and 40 bases in length, and optimally between 15 and 25 bases in length.

Oligonucleotides used as probes for in situ hybridization may be naturally occurring double stranded or single stranded DNA or RNA. Alternatively, oligonucleotides may be chemically synthesized as described above or obtained from any of a number of commercial suppliers of custom oligonucleotides. It should be understood that oligonucleotides used for in situ hybridization probes may contain modifications as described above for polynucleotides.

For purposes of hybrid detection, probes are radioactively labeled by methods well known in the art. Particularly useful is ³⁵S labeling, which combines a high energy signal with high resolution. Alternatively, a hybrid is detected via non-isotopic methods. Non-isotopically labeled probes are produced by the addition of biotin or digoxigenin, fluorescent groups, chemiluminescent groups (e.g., dioxetanes, particularly triggered dioxetanes), enzymes or antibodies. Typically, non-isotopic probes are detected by fluorescence or enzymatic methods. Detection of a radiolabeled probe-target nucleic acid complex is accomplished by separating the complex from free probe and measuring the level of complex by autoradiography. If the probe is covalently linked to an enzyme, the enzyme-probe-conjugate-target nucleic acid complex will be isolated away from the free probe enzyme conjugate and a substrate will be added for enzyme detection. Enzymatic activity will be observed as a change in color development or luminescent output resulting in a 103-106-fold increase in sensitivity. An example of the preparation and use of nucleic acid probe-enzyme conjugates as hybridization probes (wherein the enzyme is alkaline phosphatase), is described in Jablonski et al., 1986, Nuc. Acids Res., 14:6115.

Two-step label amplification methodologies are known in the art. These assays are based on the principle that a small ligand (such as digoxigenin, biotin, or the like) is attached to a nucleic acid probe capable of specifically binding to a desired target sequence.

According to the method of two-step label amplification, the small ligand attached to the nucleic acid probe will be specifically recognized by an antibody-enzyme conjugate. For example, digoxigenin will be attached to the nucleic acid probe and hybridization will be detected by an antibody-alkaline phosphatase conjugate wherein the alkaline phosphatase reacts with a chemiluminescent substrate. For methods of preparing nucleic acid probe-small ligand conjugates, see Martin et al., 1990, BioTechniques, 9:762. Alternatively, the small ligand will be recognized by a second ligand-enzyme conjugate that is capable of specifically complexing to the first ligand. A well known example of this manner of small ligand interaction is the biotin-avidin interaction. Methods for labeling nucleic acid probes and their use in biotin-avidin based assays are described in Rigby et al., 1977, J. Mol. Biol., 113:237 and Nguyen et al., 1992, BioTechniques, 13:116).

Variations of the basic hybrid detection protocol are known in the art, and include modifications that facilitate separation of the hybrids to be detected from extraneous materials and/or that employ the signal from the labeled moiety. A number of these modifications are reviewed in: Matthews & Kricka, 1988, Anal. Biochem., 169:1; Landegren et al., 1988, Science, 242:229; Mittlin, 1989, Clinical Chem. 35:1819; U.S. Pat. No. 4,868,105; and in EPO Publication No. 225,807.

As an alternative to oligonucleotides, probes for in situ hybridization analysis may comprise fragments of single or double-stranded DNA or RNA comprising sequence which allows specific hybridization with a desired target sequence. As used in this context, a “fragment” refers to any polynucleotide greater in length than an oligonucleotide, up to approximately 6 kb, but preferably between approximately 100 bases and 1 kb in length. Such fragments to be used as probes can be chemically synthesized, but are preferably generated enzymatically. It should be understood that any or all of the modifications discussed above for polynucleotides can be incorporated into such fragments.

DNA probe fragments are generated enzymatically in a number of ways. For example, fragments are generated by digestion of naturally occurring DNA or of cloned recombinant DNA bearing the desired polynucleotide sequence with restriction endonucleases according to methods well known in the art. Such restriction digest-generated sequence fragments may contain, in addition to the sequences corresponding to (i.e., complementary to) the desired target sequence, sequences derived from the surrounding genomic sequence or from the recombinant DNA vector from which it was digested. Such restriction digest-generated sequence fragments are utilized as probes following labeling by means known in the art, such as radioactive labeling or non-isotopic labeling methods as discussed above for oligonucleotide probes. Further, there may be one or many label moieties incorporated per probe molecule, depending upon the method utilized to incorporate such label. For example, DNA or RNA polynucleotides may be labeled with a single labeling moiety per molecule, as in 5′ end labeling with γ-³²P-ATP and T4 polynucleotide kinase, or with multiple ³²P-labeled bases, as in random-primed labeling with γ-³²P-labeled deoxynucleotides and the Klenow fragment of E. coli DNA Pol I.

Another means of generating longer polynucleotide fragments for use as probes in in situ hybridization involves the use of PCR techniques. Probe sequences generated by PCR methodology are isotopically or non-isotopically labeled according to methods known in the art, as discussed above. Alternatively, PCR-generated probes are labeled during the PCR process by incorporation of one or more isotopically or non-isotopically labeled nucleoside triphosphates (or analogs) added to the reaction mixture.

It should be noted that the efficiency of in situ hybridization can be enhanced when using probes derived from longer DNA or RNA fragments by partial hydrolysis of the labeled probe preparation, usually by alkali treatment. In this context, “partial hydrolysis” is meant to be hydrolysis which results in the majority of fragments generated being shorter than the length prior to hydrolysis, but greater than or equal to a length that allows specific hybridization under a given set of hybridization conditions. Preferably, the hydrolyzed probe is 8 to 500 bases long, and more preferably 20 to 200 nucleotides in length. Thus, probes derived from longer DNA or RNA fragments may in practice comprise many DNA or RNA fragments generated from them.

Another alternative means of generating probes to detect the presence of polynucleotide bearing the sequence disclosed is through in vitro transcription of a DNA template to generate the corresponding RNA. This has the advantage that it generates a single-stranded probe which will hybridize with only the sense or the antisense strand of the target nucleic acid. The use of antisense RNA probes to detect sense RNA in situ has the added advantage that RNA:RNA hybrids are generally more stable than DNA:RNA hybrids.

There are at least two ways to make the transcription template. First, the sequence to be used to generate the probe can be inserted, using vectors and techniques known in the art, into a plasmid vector adjacent to a bacteriophage promoter (usually SP6, T7, or T3). Alternatively, the bacteriophage promoter sequence may be appended to a probe template fragment by being incorporated into the 5′ end of a PCR primer, the remainder of which is complementary to one end of a sequence used as a PCR template (which contains the desired target polynucleotide sequence). PCR is then carried out using the primer with the appended promoter, an appropriate 3′ primer, and a PCR template containing the desired polynucleotide sequence to be used as probe, to generate a PCR product bearing the bacteriophage promoter at one end. It is important to note that for the initial PCR cycles (i.e., cycles 1-5), the annealing temperature chosen is based upon the calculated Tₘ of that portion of the 5′ primer which is able to hybridize with the PCR template. In subsequent cycles, the annealing temperature is adjusted (increased) to reflect the Tₘ of the fall length of the 5′ primer, which hybridizes along its full length with molecules synthesized in the initial five cycles. The promoter is situated with respect to the desired polynucleotide transcription template sequence such that either a sense or an antisense RNA transcript is generated. (It is possible to generate a template with a different bacteriophage promoter at each end, allowing the synthesis of sense and antisense transcripts from the same template, if desired).

An RNA transcript useful as a probe for in situ hybridization analysis may be generated for example, by the steps of: 1) denaturing the DNA template strands; 2) adding appropriate labeled or non-labeled ribonucleoside triphosphates or analogues thereof along with the appropriate buffers and bacteriophage RNA polymerase; 3) incubating for the appropriate time at the appropriate temperature; and 4) removal of the DNA template by digestion with DNaseI. (For specifics, see Ausubel et al., 1992, supra, pp. 14-16 to 14-17). As noted for longer DNA probes, RNA probes can be partially hydrolyzed prior to use to improve the efficiency of hybridization.

The use of a disclosed nucleotide sequence as an in situ hybridization probe to detect the presence or absence of nucleic acid corresponding (i.e. complementary) to the disclosed polynucleotide sequence in a cell, tissue, or other sample preparation, involves the steps of: 1) incubation, in the appropriate buffer, of labeled probe(s) with cells, tissue sections, or other sample preparations immobilized on glass slides or other appropriate support; 2) removal of unbound probe molecules; and 3) detection of bound probe complexes. The methods of preparation (i.e., sectioning, fixation, and pre-hybridization blocking) of cell, tissue, or other samples for in situ hybridization vary widely depending upon the characteristics of a given sample type and the form of probe to be used.

The following are examples of conditions for the preparation of histological samples. However, one skilled in the art may choose conditions appropriate for a given cell, tissue, or sample type. Tissue samples intended for use in in situ detection of either RNA or protein are fixed using conventional reagents; such samples may comprise whole or squashed cells, or may instead comprise sectioned tissue. Fixatives adequate for such procedures include, but are not limited to, fonnalin, 4% paraformaldehyde in an isotonic buffer, formaldehyde (each of which confers a measure of RNase resistance to the nucleic acid molecules of the sample) or a multi-component fixative, such as FAAG (85% ethanol, 4% formaldehyde, 5% acetic acid, 1% EM grade glutaraldehyde). Note that for RNA detection, water used in the preparation of an aqueous component of a solution to which the tissue is exposed until it is embedded is RNAase-free, i.e., treated with 0.1% diethylpyrocarbonate (DEPC) at room temperature overnight and subsequently autoclaved for 1.5 to 2 hours. Tissue is fixed at 4° C., either on a sample roller or a rocking platform, for 12 to 48 hours in order to allow fixative to reach the center of the sample.

Prior to embedding, samples are purged of fixative and dehydrated; this is accomplished through a series of two- to ten-minute washes in increasingly high concentrations of ethanol, beginning at 60% and ending with two washes each in 95% and 100% ethanol, followed by two ten-minute washes in xylene. Samples are embedded in one of a variety of sectioning supports, e.g., paraffin, plastic polymers or a mixed paraffin/polymer medium (e.g., Paraplast® Plus Tissue Embedding Medium, supplied by Oxford Labware). For example, fixed, dehydrated tissue is transferred from the second xylene wash to paraffin or a paraffin/polymer resin in the liquid-phase at about 5\tilde{8}C, then replaced three to six times over a period of approximately three hours to dilute out residual xylene, followed by overnight incubation at 5\tilde{8} C under a vacuum in order to optimize infiltration of the embedding medium into the tissue. The next day, following several more changes of medium at 20 minute to 1 hour intervals also at 5\tilde{8} C, the tissue sample is positioned in a sectioning mold, the mold is surrounded by ice water and the medium is allowed to harden. Sections of 6 m thickness are taken and affixed to ‘subbed’ slides, which are those coated with a proteinaceous substrate material, usually bovine serum albumin (BSA), to promote adhesion. Other methods of fixation and embedding are also applicable for use according to the methods of the invention; examples of these are found in Humason, G. L., 1979, Animal Tissue Techniques, 4th ed. (W. H. Freeman & Co., San Francisco), as is frozen sectioning in Serrano et al., 1989, Dev. Biol. 132:410.

In addition to variations in the fixation and sample preparation conditions, the actual procedures and conditions for the hybridization of the probe to the sample will vary according to how the tissue/cell sample was prepared.

It is contemplated that probes derived from the sequence of the present invention may be used to detect the presence of DNA or RNA complementary in sequence to the gene sequence comprising the polynucleotide of the invention in any one or more of the following cell and tissue types: muscle, including but not limited to skeletal, cardiac, and smooth muscle; cells and tissues of the circulatory system, including but not limited to those of veins and arteries; cells of the skin; bone and bone forming cells; neuronal cells and tissues of the central nervous system, including but not limited to those of the brain and spine; liver cells, including but not limited to parenchymal and non-parenchymal hepatic cells; cells and tissues of the alimentary tract, including but not limited to esophagus, stomach, large and small intestines and rectum; tissues and cells of the reproductive systems, including but not limited to those of the ovary and testis, uterus, cervix, breast and prostate; cells and tissues of the genitourinary tract, including but not limited to kidney and bladder; cells and tissues of the endocrine system, including but not limited to the adrenal glands, hypothalamus, pituitary gland, and pancreas; immune system components, including but not limited to cells of bone marrow, lymphoid and myeloid lineages, B cells, T cells, NK cells, macrophages, and cells of the spleen; cells and tissues of the pulmonary system and lung; cells of the eye and cells of the auditory system.

Alternatively, nucleic acid probes constructed as described above may be employed in a Northern Blot analysis to detect nucleic acid sequences of the present invention. Molecular methods such as Northern analysis are well known in the art (see Sambrook et al., 1989, Molecular Cloning. A Laboratory Manual., 2nd Edition, Cold Spring Harbor Laboratory Press, Cold Spring Harbor, N.Y.). Briefly, Northern analysis may be performed on total RNA obtained from one or more cells to be examined for the presence of nucleic acid encoding the GPCR of the invention using classically described techniques. For example, total RNA samples are denatured with formaldehyde/formamide and run for two hours in a 1% agarose, MOPS-acetate-EDTA gel. RNA is then transferred to nitrocellulose membrane by upward capillary action and fixed by UV cross-linkage. Membranes are pre-hybridized for at least 90 minutes and hybridized overnight at 42° C. Post hybridization washes are performed as known in the art (Ausubel, supra). The membrane is then exposed to x-ray film overnight with an intensifying screen at −80° C. Labeled membranes are then visualized after exposure to film. The signal produced on the x-ray film by the radiolabeled cDNA probes can then be quantified using any technique known in the art, such as scanning the film and quantifying the relative pixel intensity using a computer program such as NIH Image (National Institutes of Health, Bethesda, Md.).

C. Qualitative Assays

Assays that evaluate whether or not a GPCR polypeptide or the mRNA encoding it are wild-type or not can be used diagnostically. In order to diagnose a disease or disorder characterized by GPCR dysregulation in this manner, RNA isolated from a sample is used as a template for PCR amplification of GPCR. The amplified sequences are then either directly sequenced using standard methods, or are first cloned into a vector, followed by sequencing. A difference in the sequence that changes one or more encoded amino acids relative to the sequence of wild-type GPCR can be diagnostic of a disease or disorder characterized by dysregulation of GPCR signaling. It can be useful, when a change in coding sequence is identified in a sample, to express the variant receptor or ligand and compare its activity to that of wild type GPCR (i.e., wild-type GPCRx11). Among other benefits, this approach can provide novel mutants, including constitutively active and null mutants.

In addition to standard sequencing methods, amplified sequences can be assayed for the presence of specific mutations using, for example, hybridization of molecular beacons that discriminate between wild type and variant sequences. Hybridization assays that discriminate on the basis of changes as small as one nucleotide are well known in the art. Alternatively, any of a number of “minisequencing” assays can be performed, including, those described, for example, in U.S. Pat. Nos. 5,888,819, 6,004,744 and 6,013,431 (incorporated herein by reference). These assays and others known in the art can determine the presence, in a given sample, of a nucleic acid with a known polymorphism.

If desired, array or microarray-based methods can be used to analyze the expression or the presence of mutation, in GPCR sequences. Array-based methods for minisequencing and for quantitation of nucleic acid expression are well known in the art.

C. Functional Assays

Diagnosis of a disease or disorder characterized by the dysregulation of GPCR signaling can also be performed using functional assays. To do so, cell membranes or cell extracts prepared from a tissue sample are used in an assay of GPCR activity as described herein (e.g., ligand binding assays, the GTP-binding assay, GTPase assay, adenylate cyclase assay, cAMP assay, arachidonic acid level, phospholipid breakdown, diacyl glycerol or inositol triphosphate assays, PKC activation assay, or kinase assay). The activity detected is compared to that in a standard sample taken from a healthy individual or from an unaffected site on the affected individual. As an alternative, a sample or extract of a sample can be applied to cells expressing a GPCR, followed by measurement of the GPCR signaling activity relative to a standard sample. A difference of 10% or more in the activity measured in any of these assays, relative to the activity of the standard, is diagnostic for a disease or disorder characterized by dysregulation of GPCR signaling.

Modulation of GPCRx11 Activity in a Cell According to the Invention

The discovery of angiopeptin as a ligand of GPCRx11 provides methods of modulating the activity of a GPCRx11 polypeptide in a cell. GPCRx11 activity is modulated in a cell by delivering to that cell an agent that modulates the function of a GPCRx11 polypeptide. This modulation can be performed in cultured cells as part of an assay for the identification of additional modulating agents, or, for example, in an animal, including a human. Agents include angiopeptin as well as additional modulators identified using the screening methods described herein including but not limited to any of the angiopeptin analogues.

An agent can be delivered to a cell by adding it to culture medium. The amount to deliver will vary with the identity of the agent and with the purpose for which it is delivered. For example, in a culture assay to identify antagonists of GPCRx11 activity, one will preferably add an amount of agent, e.g., angiopeptin that half-maximally activates the receptors (e.g., approximately EC₅₀), preferably without exceeding the dose required for receptor saturation. This dose can be determined by titrating the amount of angiopeptin to determine the point at which further addition of angiopeptin has no additional effect on GPCRx11 activity.

When a modulator of GPCRx11 activity is administered to an animal for the treatment of a disease or disorder, the amount administered can be adjusted by one of skill in the art on the basis of the desired outcome. Successful treatment is achieved when one or more measurable aspects of the pathology (e.g., tumor cell growth, accumulation of inflammatory cells) is changed by at least 10% relative to the value for that aspect prior to treatment.

Candidate Modulators Useful According to the Invention

The invention provides for a compound that is a modulator of a receptor of the invention.

Preferably a candidate modulator is angiopeptin.

The candidate compound can be a synthetic compound, or a mixture of compounds, or may be a natural product (e.g. a plant extract or culture supernatant). A candidate compound according to the invention includes but is not limited to a small molecule that can be synthesized, a natural extract, peptides, polypeptides, carbohydrates, lipids, an antibody or antigen-binding fragment thereof, nucleic acids, and a small organic molecules.

Candidate modulator compounds from large libraries of synthetic or natural compounds can be screened. Numerous means are currently used for random and directed synthesis of saccharide, peptide, and nucleic acid based compounds. Synthetic compound libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Combinatorial libraries are available and can be prepared. Alternatively, libraries of natural compounds in the form of bacterial, fungal, plant and animal extracts are available from e.g., Pan Laboratories (Bothell, Wash.) or MycoSearch (N.C.), or are readily produceable by methods well known in the art. Additionally, natural and synthetically produced libraries and compounds are readily modified through conventional chemical, physical, and biochemical means.

Useful compounds may be found within numerous chemical classes. Useful compounds may be organic compounds, or small organic compounds. Small organic compounds have a molecular weight of more than 50 yet less than about 2,500 daltons, preferably less than about 750, more preferably less than about 350 daltons. Exemplary classes include heterocycles, peptides, saccharides, steroids, and the like. The compounds may be modified to enhance efficacy, stability, pharmaceutical compatibility, and the like. Structural identification of an agent may be used to identify, generate, or screen additional agents. For example, where peptide agents are identified, they may be modified in a variety of ways to enhance their stability, such as using an unnatural amino acid, such as a D-amino acid, particularly D-alanine, by functionalizing the amino or carboxylic terminus, e.g. for the amino group, acylation or alkylation, and for the carboxyl group, esterification or amidification, or the like.

For primary screening, a useful concentration of a candidate compound according to the invention is from about 10 μM to about 100 μM or more (i.e. 1 mM, 10 mM, 100 mM, or even 1M). The primary screening concentration will be used as an upper limit, along with nine additional concentrations, wherein the additional concentrations are determined by reducing the primary screening concentration at half-log intervals (e.g. for 9 more concentrations) for secondary screens or for generating concentration curves.

High Throughput Screening Kit

A high throughput screening kit according to the invention comprises all the necessary means and media for performing the detection of a modulator compound including an agonist, antagonist, inverse agonist or inhibitor to the receptor of the invention in the presence of propionate, preferably at a concentration in the range of 1 μM to 1 mM. The kit comprises materials to perform the following successive steps. Recombinant cells of the invention, comprising and expressing the nucleotide sequence encoding any one or more of the GPCRs disclosed herein, are grown on a solid support, such as a microtiter plate, more preferably a 96 well microtiter plate, according to methods well known to the person skilled in the art, especially as described in WO 00/02045. Modulator compounds according to the invention, at concentrations from about 1 μM to 1 mM or more, are added to the culture media of defined wells in the presence of an appropriate concentration of propionate (preferably in the range of 1 μM to 1 μM).

Kits according to the invention can also comprise materials necessary for second messenger assays amenable to high throughput screening analysis, including but not limited to the measurement of intracellular levels of cAMP, intracellular inositol phosphate, intracellular diacylglycerol concentrations, arachinoid acid concentration or MAP kinase or tyrosine kinase activity (as described above). For example, the GPCRx11 activity, as measured in a cyclic AMP assay, is quantified by a radioimmunoassay as previously described (26). Results are compared to the baseline level of GPCRx11 activity obtained from recombinant cells according to the invention in the presence of angiopeptin but in the absence of added modulator compound. Wells showing at least 2 fold, preferably 5 fold, more preferably 10 fold and most preferably a 100 fold or more increase or decrease in GPCRx11 activity as compared to the level of activity in the absence of modulator, are selected for further analysis.

Other Kits Useful According to the Invention

The invention provides for kits useful for screening for modulators of GPCR activity, as well as kits useful for diagnosis of diseases or disorders characterized by dysregulation of GPCR signaling. Kits useful according to the invention can include an isolated GPCR polypeptide (including a membrane-or cell-associated GPCR polypeptide, e.g., on isolated membranes, cells expressing GPCR, or on an SPR chip). A kit can also comprise an antibody specific for a GPCR. Alternatively, or in addition, a kit can contain cells transformed to express a GPCR polypeptide. In a further embodiment, a kit according to the invention can contain a polynucleotide encoding a GPCR polypeptide. In a still further embodiment, a kit according to the invention may comprise the specific primers useful for amplification of GPCRs as described below. All kits according to the invention will comprise the stated items or combinations of items and packaging materials therefor. Kits will also include instructions for use.

Transgenic Animals

Transgenic mice provide a useful tool for genetic and developmental biology studies and for the determination of the function of a novel sequence. According to the method of conventional transgenesis, additional copies of normal or modified genes are injected into the male pronucleus of the zygote and become integrated into the genomic DNA of the recipient mouse. The transgene is transmitted in a Mendelian manner in established transgenic strains. Constructs useful for creating transgenic animals comprise genes under the control of either their normal promoters or an inducible promoter, reporter genes under the control of promoters to be analyzed with respect to their patterns of tissue expression and regulation, and constructs containing dominant mutations, mutant promoters, and artificial fusion genes to be studied with regard to their specific developmental outcome. Typically, DNA fragments on the order of 10 kilobases or less are used to construct a transgenic animal (Reeves, 1998, New. Anat., 253:19). Transgenic animals can be created with a construct comprising a candidate gene containing one or more polymorphisms according to the invention. Alternatively, a transgenic animal expressing a candidate gene containing a single polymorphism can be crossed to a second transgenic animal expressing a candidate gene containing a different polymorphism and the combined effects of the two polymorphisms can be studied in the offspring animals.

Other Transgenic Animals

The invention provides for transgenic animals that include but are not limited to transgenic mice, rabbits, rats, pigs, sheep, horses, cows, goats, etc. A protocol for the production of a transgenic pig can be found in White and Yannoutsos, Current Topics in Complement Research: 64^{th} Forum in Immunology, pp. 88-94; U.S. Pat. Nos. 5,523,226; 5,573,933: PCT Application WO93/25071; and PCT Application WO95/04744. A protocol for the production of a transgenic mouse can be found in U.S. Pat. No. 5,530,177. A protocol for the production of a transgenic rat can be found in Bader and Ganten, Clinical and Experimental Pharmacology and Physiology, Supp. 3:S81-S87, 1996. A protocol for the production of a transgenic cow can be found in Transgenic Animal Technology, A Handbook, 1994, ed., Carl A. Pinkert, Academic Press, Inc. A protocol for the production of a transgenic rabbit can be found in Hammer et al., Nature 315:680-683, 1985 and Taylor and Fan, Frontiers in Bioscience 2:d298-308, 1997.

Knock Out Animals

i. Standard

Knock out animals are produced by the method of creating gene deletions with homologous recombination. This technique is based on the development of embryonic stem (ES) cells that are derived from embryos, are maintained in culture and have the capacity to participate in the development of every tissue in the mouse when introduced into a host blastocyst. A knock out animal is produced by directing homologous recombination to a specific target gene in the ES cells, thereby producing a null allele of the gene. The potential phenotypic consequences of this null allele (either in heterozygous or homozygous offspring) can be analyzed (Reeves, supra).

ii. In vivo Tissue Specific Knock Out in Mice Using Cre-lox

The method of targeted homologous recombination has been improved by the development of a system for site-specific recombination based on the bacteriophage PI site specific recombinase Cre. The Cre-loxP site-specific DNA recombinase from bacteriophage P1 is used in transgenic mouse assays in order to create gene knockouts restricted to defined tissues or developmental stages. Regionally restricted genetic deletion, as opposed to global gene knockout, has the advantage that a phenotype can be attributed to a particular cell/tissue (Marth, 1996, Clin. Invest. 97: 1999). In the Cre-loxP system one transgenic mouse strain is engineered such that loxP sites flank one or more exons of the gene of interest. Homozygotes for this so called ‘floxed gene’ are crossed with a second transgenic mouse that expresses the Cre gene under control of a cell/tissue type transcriptional promoter. Cre protein then excises DNA between loxP recognition sequences and effectively removes target gene function (Sauer, 1998, Methods, 14:381). There are now many in vivo examples of this method, including the inducible inactivation of mammary tissue specific genes (Wagner et al., 1997, Nucleic Acids Res., 25:4323).

iii. Bac Rescue of Knock Out Phenotype

In order to verify that a particular genetic polymorphism/mutation is responsible for altered protein function in vivo one can “rescue” the altered protein function by introducing a wild-type copy of the gene in question. In vivo complementation with bacterial artificial chromosome (BAC) clones expressed in transgenic mice can be used for these purposes. This method has been used for the identification of the mouse circadian Clock gene (Antoch et al., 1997, Cell 89: 655).

Pharmaceutical Compositions

The invention provides for compositions comprising a GPCR modulator according to the invention admixed with a physiologically compatible carrier. As used herein, “physiologically compatible carrier” refers to a physiologically acceptable diluent such as water, phosphate buffered saline, or saline, and further may include an adjuvant, and which is not toxic, carcinogenic, or caustic to an animal to which it is administered. Adjuvants such as incomplete Freund's adjuvant, aluminium phosphate, aluminium hydroxide, or alum are materials well known in the art. A “physiologically compatible carrier”, as used herein does not include culture medium, or diluents which include serum.

The invention also provides for pharmaceutical compositions. In addition to the active ingredients, these pharmaceutical compositions may contain suitable pharmaceutically acceptable carrier preparations which can be used pharmaceutically.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl cellulose, hydroxypropylmethyl-cellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hank's solution, Ringer' solution, or physiologically buffered saline. Aqueous injection suspensions may contain substances which increase the viscosity of the suspension, such as sodium carboxymnethyl cellulose, sorbitol, or dextran. Additionally, suspensions of the active solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For nasal administration, penetrants appropriate to the particular barrier to be permeated are used in the formulation. Such penetrants are generally known in the art.

The pharmaceutical compositions of the present invention may be manufactured in a manner known in the art, e.g. by means of conventional mixing, dissolving, granulating, drageemaking, levitating, emulsifying, encapsulating, entrapping or lyophilizing processes.

The pharmaceutical composition may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

After pharmaceutical compositions comprising a compound of the invention formulated in a acceptable carrier have been prepared, they can be placed in an appropriate container and labeled for treatment of an indicated condition with information including amount, frequency and method of administration.

Dosage and Mode of Administration

By way of example, a patient can be treated as follows by the administration of a modulator of GPCRx11 (for example, an agonist, antagonist or inhibitor of GPCRx11, of the invention). A modulator of a GPCR the invention can be administered to the patient, preferably in a biologically compatible solution or a pharmaceutically acceptable delivery vehicle, by ingestion, injection, inhalation or any number of other methods as described above. For administration to animals, and particularly humans, it is expected that the daily dosage level of the substance will be from 0.01 mg/kg to 10 mg/kg, typically around 1 mg/kg. The physician in any event will determine the actual dosage which will be most suitable for an individual and will vary with the age, weight and response of the particular individual. The above dosages are exemplary of the average case. There can, of course, be individual instances where higher or lower dosage ranges are merited, and such are within the scope of this invention. The dosages administered will vary from patient to patient; a “therapeutically effective dose” can be determined, for example, by the level of enhancement of function (e.g., as determined in a second messenger assay described herein). Monitoring angiopeptin binding will also enable one skilled in the art to select and adjust the dosages administered. The dosage of a modulator of a GPCR of the invention may be repeated daily, weekly, monthly, yearly, or as considered appropriate by the treating physician.

### EXAMPLES

The invention is illustrated by the following non-limiting examples wherein the following materials and methods are employed. The entire disclosure of each of the litterature references cited hereinafter is incorporated by reference herein.

### Example 1

### Cloning, Sequencing and Alignment of GPCRx11

In order to identify and clone novel human GPCR (G-protein coupled receptor) the following approach was used. Sequences of the following GPCR: GPR8, ChemR23, HM74 and GPR14 were used as queries to search for homologies in public high-throughput genomic sequence databases (NCBI).

Using the above strategies, a novel human sequence of GPCR was identified. The new GPCR was labeled: GPCRx11 (SEQ ID number 11 and 12).

In order to clone the GPCRx11 sequence we performed a polymerase chain reaction (PCR) on total human genomic DNA. Primers were synthetized based upon the GPCRx11 human sequence and were as follows:

1. An isolated G-protein coupled receptor comprising an amino acid sequence which is at least 90% identical to a sequence selected from the group consisting of SEQ ID Nos. 2, 4, 6, 8, 10, 12, 14, 16, 18, 20, 22, 24, 26, and 28.

2. An isolated G-protein coupled receptor comprising an amino acid sequence selected from the group consisting of SEQ ID Nos. 2, 4, 6, 8, 10, 12, 14, 16, 18, 20, 22, 24, 26, and 28.

3. An isolated polynucleotide encoding a G-protein coupled receptor of claim 1 or 2.

4. The isolated polynucleotide of claim 3, wherein said polynucleotide encoding each of said G-protein coupled receptors of SEQ ID Nos. 2, 4, 6, 8, 10, 12, 14, 16, 18, 20, 22, 24, 26, and 28, comprises the sequence of SEQ ID Nos. 1, 3, 5, 7, 9, 11, 13, 15, 17, 19, 21, 23, 25, and 27, respectively.

5. A nucleic acid vector comprising a polynucleotide sequence selected from the group consisting of SEQ ID Nos. 1, 3, 5, 7, 9, 11, 13, 15, 17, 19, 21, 23, 25, and 27.

6. A cell comprising the nucleic acid vector of claim 5.

7. The cell of claim 6 wherein the polynucleotide sequence of said expression vector is expressed in the cell membrane of said cell.

8. A non-human mammal having a homozygous null mutation in the gene encoding a polynucleotide selected from the group consisting of SEQ ID Nos. 2, 4, 6, 8, 10, 12, 14, 16, 18, 20, 22, 24, 26, and 28.

9. A non-human mammal transgenic for a polynucleotide encoding a G-protein coupled receptor, wherein said polynucleotide is selected from the group consisting of SEQ ID Nos. 1, 3, 5, 7, 9, 11, 13, 15, 17, 19, 21, 23, 25, and 27.

10. A method of identifying an agent that modulates the function of GPCRx11, said method comprising:
(a) contacting a GPCRx11 polypeptide with angiopeptin in the presence and absence of a candidate modulator under conditions permitting the binding of said angiopeptin to said GPCRx11 polypeptide; and
(b) measuring the binding of said GPCRx11 polypeptide to said angiopeptin, wherein a decrease in binding in the presence of said candidate modulator, relative to the binding in the absence of said candidate modulator, identifies said candidate modulator as an agent that modulates the function of GPCRx11.

11. A method of detecting, in a sample, the presence of an agent that modulates the function of GPCRx11, said method comprising:
(a) contacting a GPCRx11 polypeptide with angiopeptin in the presence and absence of said sample under conditions permitting the binding of said angiopeptin to said GPCRx11 polypeptide; and
(b) measuring the binding of said GPCRx11 polypeptide to said angiopeptin, wherein a decrease in binding in the presence of said sample, relative to the binding in the absence of said sample, indicates the presence, in said sample of an agent that modulates the function of GPCRx11.

12. A method of identifying an agent that modulates the function of GPCRx11, said method comprising:
(a) contacting a GPCRx11 polypeptide with angiopeptin in the presence and absence of a candidate modulator; and
(b) measuring a signaling activity of said GPCRx11 polypeptide, wherein a change in the activity in the presence of said candidate modulator relative to the activity in the absence of said candidate modulator identifies said candidate modulator as an agent that modulates the function of GPCRx11.

13. A method of identifying an agent that modulates the function of GPCRx11, said method comprising:
(a) contacting a GPCRx11 polypeptide with a candidate modulator;
(b) measuring a signaling activity of said GPCRx11 polypeptide in the presence of said candidate modulator; and
(c) comparing said activity measured in the presence of said candidate modulator to said activity measured in a sample in which said GPCRx11 polypeptide is contacted with angiopeptin at its EC₅₀, wherein said candidate modulator is identified as an agent that modulates the function of GPCRx11 when the amount of said activity measured in the presence of said candidate modulator is at least 20% of the amount induced by said angiopeptin present at its EC₅₀.

14. A method of detecting the presence, in a sample, of an agent that modulates the function of GPCRx11, said method comprising:
(a) contacting a GPCRx11 polypeptide with angiopeptin in the presence and absence of said sample;
(b) measuring a signaling activity of said GPCRx11 polypeptide; and
(c) comparing the amount of said activity measured in a reaction containing GPCRx11 and angiopeptin without said sample to the amount of said activity measured in a reaction containing GPCRx11, angiopeptin and said sample, wherein a change in said activity in the presence of said sample relative to the activity in the absence of said sample indicates the presence, in said sample, of an agent that modulates the function of GPCRx11.

15. A method of detecting the presence, in a sample, of an agent that modulates the function of GPCRx11, said method comprising:
(a) contacting a GPCRx11 polypeptide with said sample;
(b) measuring a signaling activity of said GPCRx11 polypeptide in the presence of said sample; and
(c) comparing said activity measured in the presence of said sample to said activity measured in a reaction in which said GPCRx11 polypeptide is contacted with angiopeptin present at its EC₅₀, wherein an agent that modulates the function of GPCRx11 is detected if the amount of said activity measured in the presence of said sample is at least 20% of the amount induced by said angiopeptin present at its EC₅₀.

16. The method of any one of claims 1-6 wherein said angiopeptin is detectably labeled.

17. The method of claim 16 wherein said angiopeptin is detectably labeled with a moiety selected from the group consisting of a radioisotope, a fluorophore, a quencher of fluorescence, an enzyme, and an affinity tag.

18. The method of any one of claims 10-15 wherein said contacting is performed in or on a cell expressing said GPCRx11 polypeptide.

19. The method of any one of claims 10-15 wherein said contacting is performed in or on synthetic liposomes.

20. The method of any one of claims 10-15 wherein said contacting is performed in or on virus-induced budding membranes containing a GPCRx11 polypeptide.

21. The method of any one of claims 10-15 wherein said method is performed using a membrane fraction from cells expressing said GPCRx11 polypeptide.

22. The method of either of claims 10 or 11 wherein said measuring is performed using a method selected from label displacement, surface plasmon resonance, fluorescence resonance energy transfer, fluorescence quenching, and fluorescence polarization.

23. The method of any one of claims 10-15 wherein said agent is selected from the group consisting of a peptide, a polypeptide, an antibody or antigen-binding fragment thereof, a lipid, a carbohydrate, a nucleic acid, and a small organic molecule.

24. The method of any one of claims 12-15 wherein said step of measuring a signaling activity of said GPCRx11 polypeptide comprises detecting a change in the level of a second messenger.

25. The method of any one of claims 12-15 wherein the step of measuring a signaling activity comprises measurement of guanine nucleotide binding or exchange, adenylate cyclase activity, cAMP, Protein Kinase C activity, phosphatidylinosotol breakdown, diacylglycerol, inositol triphosphate, intracellular calcium, arachinoid acid, MAP kinase activity, tyrosine kinase activity, or reporter gene expression.

26. The method of claim 25 wherein said measuring a signaling activity comprises using an aequorin-based assay.

27. A method of identifying a molecule which binds to a G-protein coupled receptor comprising:
(a) contacting a G-protein coupled receptor having an amino acid sequence selected from the group consisting of SEQ ID Nos. 2, 4, 6, 8, 10, 12, 14, 16, 18, 20, 22, 24, 26, and 28, with a candidate molecule;
(b) measuring the binding of said G-protein coupled receptor to said candidate molecule, wherein a detectable level of binding indicates that said candidate molecule binds to said G-protein coupled receptor.

28. The method of claim 27, wherein said candidate molecule is detectably labeled.

29. The method of claim 28, wherein said candidate molecule is detectably labeled with a moiety selected from the group consisting of a radioisotope, a fluorophore, a quencher of fluorescence, an enzyme, and an affinity tag.

30. The method of claim 27, wherein said measuring is performed using a method selected from label displacement, surface plasmon resonance, fluorescence resonance energy transfer, fluorescence quenching, and fluorescence polarization.

31. A method of identifying a ligand for a G-protein coupled receptor comprising:
(a) contacting a G-protein coupled receptor having an amino acid sequence selected from the group consisting of SEQ ID Nos. 2, 4, 6, 8, 10, 12, 14, 16, 18, 20, 22, 24, 26, and 28, with a candidate ligand;
(b) measuring a signaling activity of said G-protein coupled receptor in the presence of said candidate ligand, wherein a ligand for said G-protein coupled receptor is identified if an increase in said signaling activity is measured in the presence of said candidate ligand relative to said signaling activity in the absence of said candidate ligand.

32. The method of claim 31, wherein said candidate ligand is detectably labeled.

33. The method of claim 27 or 31, wherein said contacting is performed in or on a cell expressing said G-protein coupled receptor.

34. The method of claim 27 or 31, wherein said contacting is performed in or on synthetic liposomes.

35. The method of claim 27 or 31, wherein said contacting is performed in or on virus-induced budding membranes containing said G-protein coupled receptor.

36. The method of claim 27 or 31, wherein said method is performed using a membrane fraction from cells expressing said G-protein coupled receptor.

37. The method of claim 31 wherein said step of measuring a signaling activity of said G-protein coupled receptor comprises detecting a change in the level of a second messenger.

38. The method of claim 31 wherein the step of measuring a signaling activity comprises measurement of guanine nucleotide binding or exchange, adenylate cyclase activity, cAMP, Protein Kinase C activity, phosphatidylinosotol breakdown, diacylglycerol, inositol triphosphate, intracellular calcium, arachinoid acid, MAP kinase activity, tyrosine kinase activity, or reporter gene expression.

39. The method of claim 31 wherein said measuring a signaling activity comprises using an aequorin-based assay.

40. A method of modulating the activity of a GPCRx11 polypeptide in a cell, said method comprising the step of delivering to said cell an agent that modulates the activity of a GPCRx11 polypeptide, such that the activity of GPCRx11 is modulated.

41. A method of diagnosing a disease or disorder characterized by dysregulation of GPCRx11 signaling, said method comprising:
(a) isolating nucleic acid from a tissue sample;
(b) amplifying a GPCRx11 polynucleotide, using said nucleic acid as a template; and
(c) comparing the amount of amplified GPCRx11 polynucleotide produced in step (b) with a standard, wherein a difference in said amount of amplified GPCRx11 polynucleotide relative to said standard is diagnostic of a disease or disorder characterized by dysregulation of GPCRx11.

42. A composition consisting essentially of an isolated GPCRx11 polypeptide and isolated angiopeptin.

43. A kit for screening for agents that modulate the activity of GPCRx11, said kit comprising an isolated GPCRx11 polypeptide and isolated angiopeptin.

44. A kit for screening for agents that modulate the activity of GPCRx11, said kit comprising isolated angiopeptin and a cell membrane fraction comprising a GPCRx11 polypeptide.

45. A kit for screening for agents that modulate the activity of GPCRX 11, said kit comprising an isolated polynucleotide encoding a GPCRx11 polypeptide and isolated angiopeptin.

46. A kit for screening for agents that modulate the activity of GPCRx11, said kit comprising a cell transformed with a polynucleotide encoding a GPCRx11 polypeptide and angiopeptin.

47. A kit for the diagnosis of a disease or disorder characterized by dysregulation of GPCRx11 signaling, said kit comprising an isolated polynucleotide encoding a GPCRx11 polypeptide, a standard and packaging materials therefor.

48. The kit of claim 47 which further comprises angiopeptin.

49. A kit for the diagnosis of a disease or disorder characterized by dysregulation of GPCRx11 signaling, said kit comprising a cellular membrane fraction comprising a GPCRx11 polypeptide, a standard and packaging materials therefor.

50. The kit of claim 49 which further comprises angiopeptin.

51. The kit of claim 49 wherein said standard comprises a sample from an individual not affected by said disease or disorder.

52. A kit for the diagnosis of a disease or disorder characterized by dysregulation of GPCRx11 signaling, said kit comprising a cell transformed with a polynucleotide encoding a GPCRx11 polypeptide, a standard and packaging materials therefor.

53. The kit of claim 52, further comprising angiopeptin.

54. The kit of claim 53 wherein said standard comprises a sample from an individual not affected by said disease or disorder.
